(19) United States
(12) Patent Application Publication
(10) Pub. No.: US 2011/0263017 A1
(43) Pub. Date: Oct. 27, 2011

(54) TECHNIQUE FOR REGULATING REGENRATION OF TISSUE OR FAULTY OR ABNORMAL PART IN ORGAN USING NELL-1

(75) Inventors: Masanori Nakamura, Tokyo (JP); Shunichi Kuroda, Osaka (JP); Mitsuo Oda, Tokyo (JP); Mitsuori Mayahara, Tokyo (JP); Sachiyo Kenmotsu, Tokyo (JP); Koichi Igarashi, Osaka (JP); Kazunori Oie, Osaka (JP)

(73) Assignee: Showa University, Tokyo (JP)

(21) Appl. No.: 12/737,676

(22) PCT Filed: Aug. 3, 2009
(86) PCT No.: PCT/JP2009/063768

§ 371 Date: Jun. 24, 2011

(30) Foreign Application Priority Data
Aug. 4, 2008 (JP) 2008-201346

(51) Int. Cl. C12N 5/071; C07K 14/485

(52) U.S. Cl. 435/377; 530/399

(57) ABSTRACT

The object aims to form and maintain a cell, a tissue or an organ induced by differentiation. Disclosed is a composition for inducing the differentiation of a cell capable of being differentiated in a given direction to thereby produce a cell, a tissue or an organ through the further induction of the differentiation in the given direction. The composition comprises NELL-1 or a substance which can be altered so as to act as NELL-1 upon the differentiation. Also disclosed is a composition for maintaining a cell, a tissue or an organ produced by the induction of the differentiation.

### FIELD OF THE INVENTION

The present invention relates to the ectopia regeneration control technology of a cell, tissue and the organ. More particularly, the ectopia regeneration control technology of a cell using NELL-1, tissue and the organ is related to.

### BACKGROUND ART

There are many patients in need of the grafting of organs such as a liver, pancreas, a thyroid gland, the kidney, and it exists currently. For example, Ministry of Health, Labour and Welfare “is the synopses of 2006 nation health/the nutrition survey fructification”, and, as for the total of the person, 2,500,000 (15.4%) increase with a person of the diabetes mellitus of pertinence and the spare cluster estimated in comparison with approximately 18,700,000 personality, 2002. It is difficult the patient due to the intrinsic insulin deficiency does not control blood glucose by insulin self-administration of the day after day in that either, and to live a life, and there are many pancreas (a pancreas beta cell) grafting or the substitution and patients in need of the technique that it is, and it exists.

Also, the metabolism elevation that is abnormal if the thyroid hormone becomes excessive is seen, and Graves' disease is woken up. Conversely, it becomes the hypothyroidism when thyroid hormone is short. Organ transplantation to restore a thyroid gland function about not only the secretion reducing patient but also the patients of the extreme depressed metabolism that is the complications after a thyroidectomy operation performed to a surplus patient of the thyroid hormone or substitution and the technique that it is are required.

However, it is necessary to obtain an organ to transplant to really transplant an organ, and it must wait for the provision of the organ by the good will of the third party under the present conditions. There are many techniques differentiation way is forced to an undifferentiated cell, and to derive, and it exists, and a study is performed flourishingly, and research and development of the myriad are pushed forward even in the regenerative medicine field for clinical application. However, it is to cornea, cutis/mucosa, a bone, an explant level including the cartilage that it succeeds so far, and it does not succeed in providing an organ functioning normally in the body.

That NELL-1 relates to a bone morphogenetic cellular induction, the induction of the chondrogenesis cell is reported (patent document 1-4 and non-patent document 1). The bone repair by NELL-1 is originally performed in the place that there should be. However, even foreknowledge was not done without it being known at all to NELL-1 formation and/or action to maintain with various kinds of organs ectopically that there was.

To patent document 5, an amphibian ectoderm piece is preprocessed in the presence of activin, and it is transplanted, and that various kinds of tissue is formed is described in an embryo. This method intends for the Amphibia which is extremely higher in a self-repair capacity than mammalian, and, even in Amphibia, the survival rate of the post transplantation is only 30% (70% die). Thus, the method described in patent document 5 is impossible in the mammalian. In a culture of the mammalian which added TGF β or activin, there is not the report that grafting was endured.

Also, neofetus is torn off from placenta when it makes a method described in patent document 5 support Homo sapiens, and future disease is expected, and it will be processed and is physical and is impossible ethically. In this approach, there is not means to save a transplanted individual piece when a cell does abnormal growth to incorporate a grafting cell in an embryo and when it is active, and a drawback produces. Thus, the technique described in patent document 5 is not suitable for mammalian.

That a bone was formed in an organism ectopically is described in non-patent document 2 and 3 using BMP known as a bone morphogenetic factor. However, the thing which that it is formed of BMP is reported to is only a bone, and there is not the report that the cell except it, tissue, an organ were formed.

Cartilaginous tissue cut and brought down is broken down in a laboratory, and strong chondrogenesis protein is added, and it is disseminated on auricle-shaped collagen sponge, and, to non-patent document 4, that Homo sapiens ear cartilage was formed is described in the nude mice body by Homo sapiens when it is transplanted. However, it is only ear cartilage that that it is formed of chondrogenesis protein is reported, and there is not the report that the cell except it, tissue, an organ were formed.

[Patent Document 1]

International publication 2006/089023 pamphlet

[Patent Document 2]

International publication 2004/072100 pamphlet

[Patent Document 3]

International publication 2004/024893 pamphlet

[Patent Document 4]

International publication 01/024821 pamphlet

[Patent Document 5]

A Japanese Patent Laid-Open No. 2000-217,571 bulletin

[Non-Patent Document 1]

J Bone Miner Res. 2007 June, 22(6)918-30

[Non-Patent Document 2]

J Dent Res 82 (8): 581-584, 2003

[Non-Patent Document 3]

Plast. Reconstr. Surg. 108: 952, 2001

[Non-Patent Document 4]

Isogai N, Comparison of different chondrocytes for use in tissue engineering of cartilage model structures. Tissue Eng. 2006 April, 12(4)691-703

### SUMMARY OF THE INVENTION

### Problem to be Solved by the Invention

The problem of the present invention is to provide formation and/or a technique to maintain with a differentiation induced cell, the tissue which can be applied to regenerative medicine and an organ.

A differentiation induced cell, tissue transplanted in an organism and an organ are protected from immunorejection, and it is invivo, and another problem of the present invention is to provide a technique to maintain.

### Means to Solve the Problem

These inventors found that there was ability to make it differentiated more, and the cell in the differentiation stage guided to NELL-1 as a result of study zealously to solve the problem, and the present invention could be completed.

Also, that there was a work to inhibit in vivo immunological rejection was found, and, as for these inventors, NELL-1 could complete the present invention.

Such an action of NELL-1 is not known till now, and it is provided for the first time by the present invention.

In order to achieve the above mentioned object, for example, the present invention provides the following tool.

(Item 1)

The constituent including the material which is changed into it makes it differentiates, and the cell that orientation of constant differentiation was done guided, and it is a constituent to do to a more differentiated induced cell, tissue or an organ in way of the said differentiation, and to function as NELL-1 in a point in time of NELL-1 or the said differentiation.

(Item 2)

The constituent as claimed in the above item to form a more differentiated induced cell, tissue or an organ in way of the differentiation ectopically in the environment with adipose tissue and a blood vessel maintaining it.

(Item 3)

The constituent as claimed in the above item where the cell that orientation of the constant differentiation was accomplished is a somatic stem cell.

(Item 4)

The somatic stem cell is a constituent as claimed in blood forming tissue, epithelial tissue, connective tissue, muscular tissue and the above item which are an existing somatic stem cell to tissue selected than a cluster comprising the nervous tissue.

(Item 5)

The constituent as claimed in the above item which is the stem cell that orientation of the differentiation was done to blood forming tissue, epithelial tissue or connective tissue as for the somatic stem cell.

(Item 6)

The constituent as claimed in the above item which the somatic stem cell which there is to the blood forming tissue is hematopoietic stem-cell, and is the metrocyte of a blood cell having self-repair ability and multipotency.

(Item 7)

The somatic stem cell which there is to the epithelial tissue is a constituent as claimed in the above item which orientation of the differentiation is a done stem cell, and is the metrocyte of an epithelium system cell having self-repair ability and multipotency to epithelial tissue.

(Item 8)

The somatic stem cell which there is to the epithelial tissue is a constituent as claimed in gland original cells or the above item which is a cell included in the hair-root.

(Item 9)

The differentiation induced cell, tissue or the organ is a constituent as claimed in a hematopoietic system or an above item guided to differentiated to an epithelial system.

(Item 10)

A cell, the tissue which it differentiates, and were derived by the hematopoietic system or the organ is a constituent as claimed in the above item which is leucocyte, an erythrocyte, blood platelet, a macrophage selected than a cluster comprising differentiating neutrophils, eosinocyte, basophils and the lymphocyte and a blood corpuscle selected than the cluster comprising combinations thereof from hematopoietic stem-cell.

(Item 11)

A cell, the tissue which it differentiates, and were derived by the epithelial system or the organ is a constituent as claimed in an exocrine gland and the above item which are the pipe.

(Item 12)

The exocrine gland is a constituent as claimed in a perspiratory gland, sebaceous glands, an intestinal gland, a gastric gland and an above item selected than the cluster comprising salivary glands.

(Item 13)

A cell, the tissue which it differentiates, and were derived by the epithelial system or the organ is a constituent as claimed in hair, bulbus pili, hair-root or the above item which is an appendant glandular system in hair-root.

(Item 14)

A cell, the tissue which the cell that orientation of the constant differentiation was accomplished is glandular original cells, and were guided the differentiation to or the organ is a constituent as claimed in an exocrine gland and the above item which are the pipe.

(Item 15)

A cell, the tissue which the cell that orientation of the constant differentiation was accomplished is a cell included in the hair-root, and were guided the differentiation to or the organ is a constituent as claimed in hair, bulbus pili, hair-root or the above item which is an appendant glandular system in hair-root.

(Item 16)

The constituent as claimed in the item where the constituent includes NELL-1 in approximately 0.01 μg/mL or more.

(Item 17)

The constituent as claimed in the item where the constituent includes NELL-1 with about 5 μg/ml-approximately 50 μg/mL.

(Item 18)

The constituent as claimed in the above item which a cell, the tissue that the above differentiates, and it was induced an existing function to support naturally or an organ has.

(Item 19)

Differentiated with the cell that orientation of constant differentiation was accomplished, it makes guided, and it is materials to do to a more differentiated induced cell, tissue or an organ in way of the differentiation, and the said materials are an A) controlled-release anchorage and B) NELL-1 or materials including the materials which is changed into to function as NELL-1 in a point in time of the said differentiation.

(Item 20)

The materials as claimed in the above item to form a more differentiated induced cell, tissue or an organ in way of the differentiation ectopically in the environment with adipose tissue and a blood vessel maintaining it.

(Item 21)

The materials as claimed in the item where the controlled-release anchorage is an extracellular matrix.

(Item 22)

The materials as claimed in the above item where the controlled-release anchorage is selected than a cluster comprising collagen and the atelocollagen.

(Item 23)

The materials as claimed in the above item where the cell that orientation of the constant differentiation was accomplished is a somatic stem cell.

(Item 24)

The somatic stem cell is materials as claimed in blood forming tissue, epithelial tissue, connective tissue, muscular tissue and the above item which are an existing somatic stem cell to tissue selected than a cluster comprising the nervous tissue.

(Item 25)

The materials as claimed in the above item which is the somatic stem cell that there is the somatic stem cell to blood forming tissue, epithelial tissue or connective tissue.

(Item 26)

The materials as claimed in the above item which the somatic stem cell which there is to the blood forming tissue is hematopoietic stem-cell, and is the metrocyte of a blood cell having self-repair ability and multipotency.

(Item 27)

The somatic stem cell which there is to the epithelial tissue is materials as claimed in the above item which orientation of the differentiation is a done stem cell, and is the metrocyte of an epithelium system cell having self-repair ability and multipotency to epithelial tissue.

(Item 28)

The somatic stem cell which there is to the epithelial tissue is materials as claimed in gland original cells or the above item which is a cell included in the hair-root.

(Item 29)

The differentiation induced cell, tissue or the organ is materials as claimed in the hematopoietic system or epithelium above item which pro-, is a differentiated induced cell, tissue or an organ.

(Item 30)

A cell, the tissue which it differentiates, and were derived by the hematopoietic system or the organ is materials as claimed in the above item which is leucocyte, an erythrocyte, blood platelet, a macrophage selected than a cluster comprising differentiating neutrophils, eosinocyte, basophils and the lymphocyte and a blood corpuscle selected than the cluster comprising combinations thereof from hematopoietic stem-cell.

(Item 31)

A cell, the tissue which it differentiates, and were derived by the epithelial system or the organ is materials as claimed in an exocrine gland and the above item which are the pipe.

(Item 32)

The exocrine gland is materials as claimed in a perspiratory gland, sebaceous glands, an intestinal gland, a gastric gland and an above item selected than the cluster comprising salivary glands.

(Item 33)

A cell, the tissue which it differentiates, and were derived by the epithelial system or the organ is hair, bulbus pili, hair-root or materials as claimed in the above item which is an appendant glandular system in hair-root.

(Item 34)

A cell, the tissue which the cell that orientation of the constant differentiation was accomplished is glandular original cells, and were guided the differentiation to or the organ is materials as claimed in an exocrine gland and the above item which are the pipe.

(Item 35)

A cell, the tissue which the cell that orientation of the constant differentiation was accomplished is a cell included in the hair-root, and were guided the differentiation to or the organ is hair, bulbus pili, hair-root or materials as claimed in the above item which is an appendant glandular system in hair-root.

(Item 36)

The materials as claimed in the item NELL-1 is approximately 0.01 μg/mL or more, and to include.

(Item 37)

The materials as claimed in the item to include NELL-1 with about 5 μg/ml-approximately 50 μg/mL.

(Item 38)

The materials as claimed in the above item which a cell, the tissue that the above differentiates, and it was induced an existing function to support naturally or an organ has.

(Item 39)

The kit comprising the material that it makes it differentiates, and the cell that orientation of constant differentiation was accomplished guided, and it is a kit to do to a more differentiated induced cell, tissue or an organ in way of the differentiation, and the said kit is changed into to function as NELL-1 in a point in time of NELL-1 or the said differentiation.

(Item 40)

The kit as claimed in the above item to form a more differentiated induced cell, tissue or an organ in way of the differentiation ectopically in the environment with adipose tissue and a blood vessel maintaining it.

(Item 41)

The differentiation induced cell, tissue or an organ is a kit as claimed in the hematopoietic system or epithelium above item which pro-, is a differentiated induced cell, tissue or an organ.

(Item 42)

A cell, the tissue which it differentiates, and were derived by the hematopoietic system or an organ is a kit as claimed in the above item which is leucocyte, an erythrocyte, blood platelet, a macrophage selected than a cluster comprising differentiating neutrophils, eosinocyte, basophils and the lymphocyte and a blood corpuscle selected than the cluster comprising combinations thereof from hematopoietic stem-cell.

(Item 43)

A cell, the tissue which it differentiates, and were derived by the epithelial system or an organ is a kit as claimed in an exocrine gland and the above item which are the pipe.

(Item 44)

The exocrine gland is a kit as claimed in a perspiratory gland, sebaceous glands, an intestinal gland, a gastric gland and an above item selected than the cluster comprising salivary glands.

(Item 45)

A cell, the tissue which it differentiates, and were derived by the epithelial system or an organ is a kit as claimed in hair, bulbus pili, hair-root or the above item which is an appendant glandular system in hair-root.

(Item 46)

The kit as claimed in the item where NELL-1 is included in in approximately 0.01 μg/mL or more.

(Item 47)

The kit as claimed in the item where NELL-1 is included in in approximately 5 μg/mL of . . . about 50 ug/ml.

(Item 48)

Differentiated with the cell that orientation of constant differentiation was accomplished, it makes guided, and it is a medical device to do to a more differentiated induced cell, tissue or an organ in way of the differentiation, and the said medical device,

A) NELL-1 or the matter that it is changed to function as NELL-1 at a point of said differentiation,

B) A controlled-release anchorage, And

C) A container, The medical device which comprises

(Item 49)

The medical device as claimed in the above item to form a more differentiated induced cell, tissue or an organ in way of the differentiation ectopically in the environment with adipose tissue and a blood vessel maintaining it.

(Item 50)

The medical device as claimed in the item where the controlled-release anchorage is selected than the cluster comprising extracellular matrices.

(Item 51)

The medical device as claimed in the item where the controlled-release anchorage is collagen and atelocollagen.

(Item 52)

The differentiation induced cell, tissue or an organ is a medical device as claimed in the hematopoietic system or epithelium above item which pro-, is a differentiated induced cell, tissue or an organ.

(Item 53)

A cell, the tissue which it differentiates, and were derived by the hematopoietic system or an organ is a medical device as claimed in the above item which is leucocyte, an erythrocyte, blood platelet, a macrophage selected than a cluster comprising differentiating neutrophils, eosinocyte, basophils and the lymphocyte and a blood corpuscle selected than the cluster comprising combinations thereof from hematopoietic stem-cell.

(Item 54)

A cell, the tissue which it differentiates, and were derived by the epithelial system or an organ is a medical device as claimed in an exocrine gland and the above item which are the pipe.

(Item 55)

The exocrine gland is a medical device as claimed in a perspiratory gland, sebaceous glands, an intestinal gland, a gastric gland and an above item selected than the cluster comprising salivary glands.

(Item 56)

A cell, the tissue which it differentiates, and were derived by the epithelial system or an organ is a medical device as claimed in hair, bulbus pili, hair-root or the above item which is an appendant glandular system in hair-root.

(Item 57)

The medical device as claimed in the item where NELL-1 is included in in approximately 0.01 μg/mL or more.

(Item 58)

The medical device as claimed in the item where NELL-1 is included in in approximately 5 μg/mL of . . . about 50 ug/ml.

(Item 59)

It is a method to produce a cell, tissue or organs, and the said method is the following processes: A process to provide the cell that orientation of constant differentiation was accomplished, A method to include a process to make the material which is changed into to function as NELL-1 in the cell that orientation of the said constant differentiation was done and a point in time of NELL-1 or the said retention touch.

(Item 60)

The cell that orientation of the constant differentiation was accomplished is the method as claimed in the above item which environmental, is provided with adipose tissue and a blood vessel maintaining it.

(Item 61)

Cell and NELL-1 where orientation of the constant differentiation was accomplished are the methods as claimed in a touched above item on a sustained release anchorage.

(Item 62)

The method as claimed in the item where the controlled-release anchorage is selected than the cluster comprising extracellular matrices.

(Item 63)

The method as claimed in the item where the controlled-release anchorage is collagen and atelocollagen.

(Item 64)

The method as claimed in the above item where the cell that orientation of the constant differentiation was accomplished is a somatic stem cell.

(Item 65)

The somatic stem cell is a method as claimed in blood forming tissue, epithelial tissue, connective tissue, muscular tissue and the above item which are an existing somatic stem cell to tissue selected than a cluster comprising the nervous tissue.

(Item 66)

The method as claimed in the above item which is the somatic stem cell that there is the somatic stem cell to blood forming tissue, epithelial tissue or connective tissue.

(Item 67)

The method as claimed in the above item which the somatic stem cell which there is to the blood forming tissue is hematopoietic stem-cell, and is the metrocyte of a blood cell having self-repair ability and multipotency.

(Item 68)

The somatic stem cell which there is to the epithelial tissue is a method as claimed in the above item which orientation of the differentiation is a done stem cell, and is the metrocyte of an epithelium system cell having self-repair ability and multipotency to epithelial tissue.

(Item 69)

The somatic stem cell which there is to the epithelial tissue is a method as claimed in gland original cells or the above item which is a cell included in the hair-root.

(Item 70)

The constituent including the material that it is a constituent to maintain a differentiation induced cell, tissue or an organ, and the said constituent is changed into to function as NELL-1 in a point in time of NELL-1 or the said retention.

(Item 71)

The constituent as claimed in an above item performed in the place where the presence of the differentiation induced cell, tissue or the organ is inadmissible as for the retention.

(Item 72)

The constituent as claimed in the above item which a cell, the tissue that the above differentiates, and it was induced an existing function to support naturally or an organ has.

(Item 73)

The differentiation induced cell, tissue or the organ is a liver, kidney, pancreas, a constituent as claimed in the above item which adrenal, is selected than a cluster comprising a thyroid gland, an ovary mammal and the spermary.

(Item 74)

A process to provide a differentiation induced cell, tissue or a cell, the tissue that it is a method to maintain an organ, and the said method was guided the said differentiation to or an organ, A method to include a process to give the material which is changed into to function as NELL-1 in a point in time of NELL-1 or the said retention to the said differentiation induced cell, tissue or an organ.

(Item 75)

The use of the material which is changed into by a differentiation induced cell, tissue or a cell done orientation of constant differentiation with an organ to function as NELL-1 in a point in time of NELL-1 in the medical and pharmaceutical production to form or the said formation.

(Item 76)

The use of the material which is changed into to function as NELL-1 in a point in time of NELL-1 in the medical and pharmaceutical production to maintain a differentiation induced cell, tissue or an organ or the said retention.

(Item A1)

It is a method to produce in the adipose tissue which ported a glandular system in an organism or muscular tissue, and the said method is the following processes:

A) A process to manufacture a space to port a container in the said organism

B) A process the said organic adipose tissue or muscular tissue is separated to the size of the container with a blood vessel maintaining the said adipose tissue or muscular tissue in total, and to manufacture the caulescent flap including the blood vessel handle

C) A process the controlled-release false work which incorporated NELL-1 is located to the said container, and to insert the said caulescent tissue piece

D) A process to transplant the said container in the said space in the conditions where the blood flow in the said caulescent tissue piece is not obstructed

E) A method to include a process to maintain the said container in the said organism for the period that is enough so that the said glandular system is produced in the state that the blood flow of the said caulescent tissue piece is not obstructed.

(Item A2)

It is a method to produce in the adipose tissue which ported a glandular system in an organism or muscular tissue, and the said method is the following processes:

A) A process to manufacture a space to port a container in the said organism

B) A process said organic adipose tissue or muscular tissue is separated to the size of the container with a blood vessel maintaining the said adipose tissue or muscular tissue in total, and to manufacture the caulescent flap including the blood vessel handle

C) A process the said caulescent tissue piece is inserted into the said container, and to add NELL-1

D) A process to transplant the said container in the said space in the conditions where the blood flow in the said caulescent tissue piece is not obstructed

E) A method it is a process to maintain the said container in the said organism for the period that is enough so that the said glandular system is produced in the state that the blood flow of the said caulescent tissue piece is not obstructed, and to include a process to add NELL-1 regularly in the said caulescent tissue piece between the said retention.

(Item A3)

The method as claimed in the above item which is the size that the container is in the space.

(Item A4)

The method as claimed in the item where the space is manufactured between the lines in the femoral region.

(Item A5)

The method as claimed in the item where NELL-1 is contained with about 5 μg/ml-approximately 50 μg/mL.

(Item A6)

The controlled-release anchorage is the method as claimed in collagen sponge and an above item selected than the cluster comprising atelocollagen gels.

(Item A7)

It is a method to produce in the adipose tissue which ported a glandular system in a femoral region of the mammalian, and the said method is the following processes:

A) A process to exfoliate a lower abdominal wall status pulse skin incision is added to the said femoral region of the said mammalian, and to diverge from a thigh status pulse and a side-by-side travel nerve from a surrounding tissue

B) A process to manufacture the caulescent flap which abdominal regions subcutaneous fatty tissue of the said mammalian is separated to the size of the container mainly on the furcation of the lower abdominal wall status pulse in total, and assumes a lower abdominal wall status pulse a blood vessel handle

C) A process the collagen sponge which incorporated NELL-1 (about 5 μg/ml-approximately 50 μg/mL) into the said container or atelocollagen gel is placed, and to insert the said caulescent tissue piece

D) A process to transplant the said container between the lines of the said femoral region in the conditions where the blood flow in the said caulescent tissue piece is not obstructed

E) A method to include a process to maintain the said container within the said line in the state that the blood flow of the said caulescent tissue piece is not obstructed for approximately two weeks or more.

(Item A8)

It is a method to produce in the adipose tissue which ported a glandular system in a femoral region of the mammalian, and the said method is the following processes:

A) A process to exfoliate a lower abdominal wall status pulse skin incision is added to the said femoral region of the said mammalian, and to diverge from a thigh status pulse and a side-by-side travel nerve from a surrounding tissue

B) A process to manufacture the caulescent flap which abdominal regions subcutaneous fatty tissue of the said mammalian is separated to the size of the container mainly on the furcation of the lower abdominal wall status pulse in total, and assumes a lower abdominal wall status pulse a blood vessel handle

C) A process the said caulescent tissue piece is inserted into the said container, and to add NELL-1 (about 5 μg/ml-approximately 50 μg/mL)

D) A process to transplant the said container between the lines of the said femoral region in the conditions where the blood flow in the said caulescent tissue piece is not obstructed

E) A method it is a process to maintain the said container within the said line in the state that the blood flow of the said caulescent tissue piece is not obstructed for approximately two weeks or more, and to include a process to add NELL-1 (about 5 μg/ml-approximately 50 μg/mL) every other week in the said caulescent tissue piece between the said retention.

(Process A9)

The method as claimed in the item including NELL-1 is concentration of 50 μg/1 ml, and being added 20 μg/mL in total by 0.1 mL every other week for four weeks.

(Item A10)

It is a method to produce in the muscular tissue which ported a glandular system in a femoral region of the mammalian, and the said method is the following processes:

A) A process to exfoliate a lower abdominal wall status pulse skin incision is added to the said femoral region of the said mammalian, and to diverge from a thigh status pulse and a side-by-side travel nerve from a surrounding tissue

B) A process to manufacture the caulescent flap which it is separated to the size of the container in total led by a vascular branch maintaining muscular tissue of the said mammalian, and assumes the said blood vessel a handle

C) A process the collagen sponge which incorporated NELL-1 (about 5 μg/ml-approximately 50 μg/mL) into the said container or atelocollagen gel is placed, and to insert the said caulescent tissue piece

D) A process to transplant the said container between the lines of the said femoral region in the conditions where the blood flow in the said caulescent tissue piece is not obstructed

E) A method to include a process to maintain the said container within the said line in the state that the blood flow of the said caulescent tissue piece is not obstructed for approximately two weeks or more.

(Item A11)

It is a method to produce in the muscular tissue which ported a glandular system in a femoral region of the mammalian, and the said method is the following processes:

A) A process to exfoliate a lower abdominal wall status pulse skin incision is added to the said femoral region of the said mammalian, and to diverge from a thigh status pulse and a side-by-side travel nerve from a surrounding tissue

B) A process to manufacture the caulescent flap which it is separated to the size of the container in total led by a vascular branch maintaining muscular tissue of the said mammalian, and assumes the said blood vessel a handle

C) A process the said caulescent tissue piece is inserted into the said container, and to add NELL-1 (about 5 μg/ml-approximately 50 μg/mL)

D) A process to transplant the said container between the lines of the said femoral region in the conditions where the blood flow in the said caulescent tissue piece is not obstructed

E) A method it is a process to maintain the said container within the said line in the state that the blood flow of the said caulescent tissue piece is not obstructed for approximately two weeks or more, and to include a process to add NELL-1 (about 5 μg/ml-approximately 50 μg/mL) regularly in the said caulescent tissue piece between the said retention.

(Item A12)

The period that it is a method to produce in the adipose tissue which ported a glandular system in a femoral region of the mammalian, and above A) process adds skin incision to the said femoral region of the said albino rat, and it is a process to exfoliate a lower abdominal wall status pulse to diverge from a femoris status pulse and a side-by-side travel nerve from a surrounding tissue, and is enough so that above B) process can add abdominal regions subcutaneous fatty tissue of the B) said mammalian and the size of the container mainly on the furcation of the lower abdominal wall status pulse, and it is separated, and it is a process to manufacture the caulescent flap which assumes a lower abdominal wall status pulse a blood vessel handle, and above NELL-1 is about 5 μg/ml-approximately 50 μg/mL, and an above sustained release anchorage is collagen sponge or an atelocollagen gel and an above glandular system is produced in above E) process in above C) is the method as claimed in the above item for at least approximately two Week.

(Item A13)

The period that is enough for the size of the container mainly on the vascular furcation that it is a method to produce in the muscular tissue which ported a glandular system in a femoral region of the mammalian, and above A) process adds skin incision to the said femoral region of the said mammalian, and it is a process to exfoliate a lower abdominal wall status pulse to diverge from a femoris status pulse and a side-by-side travel nerve from a surrounding tissue, and above B) process maintains muscular tissue of the said mammalian so that it is separated in total, and it is a process to manufacture the caulescent flap which assumes the said blood vessel a handle, and above NELL-1 is about 5 μg/ml-approximately 50 μg/mL, and an above sustained release anchorage is collagen sponge or an atelocollagen gel and an above glandular system is produced in above E) process in above C) is the method as claimed in the above item for at least approximately two Week.

(Item A14)

It is a method to produce in the adipose tissue which ported a glandular system in a femoral region of the mammalian, and the said method is the following processes: The period that skin incision is added to the said femoral region of the said mammalian, and the A) process is a process to exfoliate a lower abdominal wall status pulse to diverge from a thigh status pulse and a side-by-side travel nerve from a surrounding tissue, and is enough so that above B) process can add abdominal regions subcutaneous fatty tissue of the B) said mammalian and the size of the container mainly on the furcation of the lower abdominal wall status pulse, and it is separated, and it is a process to manufacture the caulescent flap which assumes a lower abdominal wall status pulse a blood vessel handle, and above NELL-1 is about 5 μg/ml-approximately 50 μg/mL, and above D) process is a process to transplant the said container between the lines of the said femoral region in the state that the blood flow in the said caulescent tissue piece is not obstructed, and an above glandular system is produced in above E) process in above C) process is the method as claimed in the above item where NELL-1 includes [symbol] added with about 5 μg/ml-approximately 50 μg/mL every other week in to the said caulescent tissue piece between above retention for at least approximately two Week.

(Item A15)

The period that is enough for the size of the container mainly on the vascular furcation that it is a method to produce in the muscular tissue which ported a glandular system in a femoral region of the mammalian, and above A) process adds skin incision to the said femoral region of the said mammalian, and it is a process to exfoliate a lower abdominal wall status pulse to diverge from a femoris status pulse and a side-by-side travel nerve from a surrounding tissue, and above B) process maintains muscular tissue of the said mammalian so that it is separated in total, and it is a process to manufacture the caulescent flap which assumes the said blood vessel a handle, and above NELL-1 is about 5 μg/ml-approximately 50 μg/mL, and above D) process is a process to transplant the said container between the lines of the said femoral region in the state that the blood flow in the said caulescent tissue piece is not obstructed, and an above glandular system is produced in above E) process in above C) process is the method as claimed in the above item where NELL-1 includes added with about 5 μg/ml-approximately 50 μg/mL every other week in to the said caulescent tissue piece between above retention for at least approximately two Week.

(Item B1)

It is a method to produce in the adipose tissue which transplanted blood forming tissue in an organism or muscular tissue, and the said method is the following processes:

A) A process to manufacture a space to port a container in the said organism

B) A process the said organic adipose tissue or muscular tissue is separated to the size of the container with a blood vessel maintaining the said adipose tissue or muscular tissue in total, and to manufacture the caulescent flap including the blood vessel handle

C) A process the controlled-release false work which incorporated NELL-1 is located to the said container, and to insert the said caulescent tissue piece

D) A process to transplant the said container in the said space in the conditions where the blood flow in the said caulescent tissue piece is not obstructed

E) A method to include a process to maintain the said container in the said organism for the period that is enough so that the said blood forming tissue is produced in the state that the blood flow of the said caulescent tissue piece is not obstructed.

(Item B2)

It is a method to produce in the adipose tissue which transplanted blood forming tissue in an organism or muscular tissue, and the said method is the following processes:

A) A process to manufacture a space to port a container in the said organism

B) A process the said organic adipose tissue or muscular tissue is separated to the size of the container with a blood vessel maintaining the said adipose tissue or muscular tissue in total, and to manufacture the caulescent flap including the blood vessel handle

C) A process the said caulescent tissue piece is inserted into the said container, and to add NELL-1

D) A process to transplant the said container in the said space in the conditions where the blood flow in the said caulescent tissue piece is not obstructed

E) A method it is a process to maintain the said container in the said organism for the period that is enough so that the said blood forming tissue is produced in the state that the blood flow of the said caulescent tissue piece is not obstructed, and to include a process to add NELL-1 regularly in the said caulescent tissue piece between the said retention.

(Item B3)

The method as claimed in the above item which is the size that the container is in the space.

(Item B4)

The method as claimed in the item where the space is manufactured between the lines in the femoral region.

(Item B5)

The method as claimed in the item where NELL-1 is contained with about 5 μg/ml-approximately 50 μg/mL.

(Item B6)

The controlled-release anchorage is the method as claimed in collagen sponge and an above item selected than the cluster comprising atelocollagen gels.

(Item B7)

It is a method to produce in the adipose tissue which transplanted blood forming tissue in a femoral region of the mammalian, and the said method is the following processes:

A) A process to exfoliate a lower abdominal wall status pulse skin incision is added to the said femoral region of the said mammalian, and to diverge from a thigh status pulse and a side-by-side travel nerve from a surrounding tissue

B) A process to manufacture the caulescent flap which abdominal regions subcutaneous fatty tissue of the said mammalian is separated to the size of the container mainly on the furcation of the lower abdominal wall status pulse in total, and assumes a lower abdominal wall status pulse a blood vessel handle

C) A process the collagen sponge which incorporated NELL-1 (about 5 μg/ml-approximately 50 μg/mL) into the said container or atelocollagen gel is placed, and to insert the said caulescent tissue piece

D) A process to transplant the said container between the lines of the said femoral region in the conditions where the blood flow in the said caulescent tissue piece is not obstructed

E) The method that the blood flow of the said caulescent tissue piece includes a process to maintain mammalian within the said line in the state that is not obstructed for at least approximately two Week in with the said container.

(Item B8)

It is a method to produce in the adipose tissue which transplanted blood forming tissue in a femoral region of the mammalian, and the said method is the following processes:

A) A process to exfoliate a lower abdominal wall status pulse skin incision is added to the said femoral region of the said mammalian, and to diverge from a thigh status pulse and a side-by-side travel nerve from a surrounding tissue

B) A process to manufacture the caulescent flap which abdominal regions subcutaneous fatty tissue of the said mammalian is separated to the size of the container mainly on the furcation of the lower abdominal wall status pulse in total, and assumes a lower abdominal wall status pulse a blood vessel handle

C) A process the said caulescent tissue piece is inserted into the said container, and to add NELL-1 (about 5 μg/ml-approximately 50 μg/mL)

D) A process to transplant the said container between the lines of the said femoral region in the conditions where the blood flow in the said caulescent tissue piece is not obstructed

E) A method it is a process to maintain the said container within the said line in the state that the blood flow of the said caulescent tissue piece is not obstructed for approximately two weeks or more, and to include a process to add NELL-1 (about 5 μg/ml-approximately 50 μg/mL) every other week in the said caulescent tissue piece between the said retention.

(Process B9)

The method as claimed in the item including NELL-1 is concentration of 50 μg/1 ml, and being added 20 μg/mL in total by 0.1 mL every other week for four weeks.

(Item B10)

It is a method to produce in the muscular tissue which transplanted blood forming tissue in a femoral region of the mammalian, and the said method is the following processes:

A) A process to exfoliate a lower abdominal wall status pulse skin incision is added to the said femoral region of the said mammalian, and to diverge from a thigh status pulse and a side-by-side travel nerve from a surrounding tissue

B) A process to manufacture the caulescent flap which muscular tissue of the said mammalian is separated to the size of the container in total led by a vascular branch maintaining muscular tissue, and assumes the said blood vessel a handle

C) A process the collagen sponge which incorporated NELL-1 (about 5 μg/ml-approximately 50 μg/mL) into the said container or atelocollagen gel is placed, and to insert the said caulescent tissue piece

D) A process to transplant the said container between the lines of the said femoral region in the conditions where the blood flow in the said caulescent tissue piece is not obstructed

E) A method to include a process to maintain the said container within the said line in the state that the blood flow of the said caulescent tissue piece is not obstructed for approximately two weeks or more.

(Item B11)

It is a method to produce in the muscular tissue which transplanted blood forming tissue in a femoral region of the mammalian, and the said method is the following processes:

A) A process to exfoliate a lower abdominal wall status pulse skin incision is added to the said femoral region of the said mammalian, and to diverge from a thigh status pulse and a side-by-side travel nerve from a surrounding tissue

B) A process to manufacture the caulescent flap which it is separated to the size of the container in total led by a vascular branch maintaining muscular tissue of the said mammalian, and assumes the said blood vessel a handle

C) A process the said caulescent tissue piece is inserted into the said container, and to add NELL-1 (about 5 μg/ml-approximately 50 μg/mL)

D) A process to transplant the said container between the lines of the said femoral region in the conditions where the blood flow in the said caulescent tissue piece is not obstructed

E) A method it is a process to maintain the said container within the said line in the state that the blood flow of the said caulescent tissue piece is not obstructed for approximately two weeks or more, and to include a process to add NELL-1 (about 5 μg/ml-approximately 50 μg/mL) regularly in the said caulescent tissue piece between the said retention.

(Item B12)

The period that it is a method to produce in the adipose tissue which transplanted blood forming tissue in a femoral region of the mammalian, and above A) process adds skin incision to the said femoral region of the said albino rat, and it is a process to exfoliate a lower abdominal wall status pulse to diverge from a femoris status pulse and a side-by-side travel nerve from a surrounding tissue, and is enough so that above B) process can add abdominal regions subcutaneous fatty tissue of the B) said mammalian and the size of the container mainly on the furcation of the lower abdominal wall status pulse, and it is separated, and it is a process to manufacture the caulescent flap which assumes a lower abdominal wall status pulse a blood vessel handle, and above NELL-1 is about 5 μg/ml-approximately 50 μg/mL, and an above sustained release anchorage is collagen sponge or an atelocollagen gel and above blood forming tissue is produced in above E) process in above C) is the method as claimed in the above item for at least approximately two Week.

(Item B13)

The period that is enough for the size of the container mainly on the vascular furcation that it is a method to produce in the muscular tissue which transplanted blood forming tissue in a femoral region of the mammalian, and above A) process adds skin incision to the said femoral region of the said mammalian, and it is a process to exfoliate a lower abdominal wall status pulse to diverge from a femoris status pulse and a side-by-side travel nerve from a surrounding tissue, and above B) process maintains muscular tissue of the said mammalian so that it is separated in total, and it is a process to manufacture the caulescent flap which assumes the said blood vessel a handle, and above NELL-1 is about 5 μg/ml-approximately 50 μg/mL, and an above sustained release anchorage is collagen sponge or an atelocollagen gel and above blood forming tissue is produced in above E) process in above C) is the method as claimed in the above item for at least approximately two Week.

(Item B14)

It is a method to produce in the adipose tissue which transplanted blood forming tissue in a femoral region of the mammalian, and the said method is the following processes: A) The period that skin incision is added to the said femoral region of the said mammalian, and a process is a process to exfoliate a lower abdominal wall status pulse to diverge from a thigh status pulse and a side-by-side travel nerve from a surrounding tissue, and is enough so that above B) process can add abdominal regions subcutaneous fatty tissue of the B) said mammalian and the size of the container mainly on the furcation of the lower abdominal wall status pulse, and it is separated, and it is a process to manufacture the caulescent flap which assumes a lower abdominal wall status pulse a blood vessel handle, and above NELL-1 is about 5 μg/ml-approximately 50 μg/mL, and above D) process is a process to transplant the said container between the lines of the said femoral region in the state that the blood flow in the said caulescent tissue piece is not obstructed, and above blood forming tissue is produced in above E) process in above C) process is the method as claimed in the above item where NELL-1 includes [symbol] added with about 5 μg/ml-approximately 50 μg/mL every other week in to the said caulescent tissue piece between above retention for at least approximately two Week.

(Item B15)

The period that is enough for the size of the container mainly on the vascular furcation that it is a method to produce in the muscular tissue which transplanted blood forming tissue in a femoral region of the mammalian, and above A) process adds skin incision to the said femoral region of the said mammalian, and it is a process to exfoliate a lower abdominal wall status pulse to diverge from a femoris status pulse and a side-by-side travel nerve from a surrounding tissue, and above B) process maintains muscular tissue of the said mammalian so that it is separated in total, and it is a process to manufacture the caulescent flap which assumes the said blood vessel a handle, and above NELL-1 is about 5 μg/ml-approximately 50 μg/mL, and above D) process is a process to transplant the said container between the lines of the said femoral region in the state that the blood flow in the said caulescent tissue piece is not obstructed, and above blood forming tissue is produced in above E) process in above C) process is the method as claimed in the above item where NELL-1 includes [symbol] added with about 5 μg/ml-approximately 50 μg/mL every other week in to the said caulescent tissue piece between above retention for at least approximately two Week.

(Item C1)

It is a method to produce in the adipose tissue which transplanted a glandular system accompanying hair, bulbus pili, hair-root and hair-root in an organic femoral region or muscular tissue, and the said method is the following processes:

A) A process to manufacture a space to port a container in the said organism

B) A process the said organic adipose tissue or muscular tissue is separated to the size of the container with a blood vessel maintaining the said adipose tissue or muscular tissue in total, and to manufacture the caulescent flap including the blood vessel handle

C) A process to locate the controlled-release false work which incorporated NELL-1 to the said container,

D) A process to place a cell included in the hair-root on the said controlled-release anchorage

E) The process which inserts the said caulescent tissue piece into the said container

F) A process to transplant the said container in the state that the blood flow in the said caulescent tissue piece is not obstructed in the said space and

G) A method to include a process to maintain the said container in the said organism for the period that is enough so that an appendant glandular system is produced in the state that the blood flow of the said caulescent tissue piece is not obstructed by the said hair, bulbus pili, hair-root and hair-root.

(Item C2)

It is a method to produce in the adipose tissue which transplanted a glandular system accompanying hair, bulbus pili, hair-root and hair-root in an organic femoral region or muscular tissue, and the said method is the following processes:

A) A process to manufacture a space to port a container in the said organism

B) A process the said organic adipose tissue or muscular tissue is separated to the size of the container with a blood vessel maintaining the said adipose tissue or muscular tissue in total, and to manufacture the caulescent flap including the blood vessel handle

C) A process the said caulescent tissue piece is inserted into the said container, and to add cell included in the hair-root and NELL-1

D) A process to transplant the said container in the said space in the conditions where the blood flow in the said caulescent tissue piece is not obstructed

E) A method it is a process to maintain the said container in the said organism for the period that is enough so that an appendant glandular system is produced in the state that the blood flow of the said caulescent tissue piece is not obstructed by the said hair, bulbus pill, hair-root and hair-root, and to include a process to add NELL-1 regularly in the said caulescent tissue piece between the said retention.

(Item C3)

The method as claimed in the above item which is the size that the container is in the space.

(Item C4)

The method as claimed in the item where the space is manufactured between the lines in the femoral region.

(Item C5)

The method as claimed in the item where NELL-1 is contained with about 5 μg/ml-approximately 50 μg/mL.

(Item C6)

The controlled-release anchorage is the method as claimed in collagen sponge and an above item selected than the cluster comprising atelocollagen gels.

(Item C7)

It is a method to produce in the adipose tissue which transplanted a glandular system accompanying hair, bulbus pili, hair-root and hair-root in a femoral region of the mammalian, and the said method is the following processes:

A) A process to exfoliate a lower abdominal wall status pulse skin incision is added to the said femoral region of the said mammalian, and to diverge from a thigh status pulse and a side-by-side travel nerve from a surrounding tissue

B) A process to manufacture the caulescent flap which abdominal regions subcutaneous fatty tissue of the said mammalian is separated to the size of the container mainly on the furcation of the lower abdominal wall status pulse in total, and assumes a lower abdominal wall status pulse a blood vessel handle

C) A process to place the collagen sponge which incorporated NELL-1 (about 5μg/ml-approximately 50 μg/mL) into the said container or atelocollagen gel,

D) A process to place a cell included in the hair-root on the said collagen sponge or atelocollagen gel

E) The process which inserts a caulescent tissue piece into the said container

F) A process to transplant the said container between the lines of the said femoral region in the state that the blood flow in the said caulescent tissue piece is not obstructed and

G) A method to include a process to maintain the said container within the said line in the state that the blood flow of the said caulescent tissue piece is not obstructed for approximately two weeks or more.

(Item C8)

It is a method to produce in the adipose tissue which transplanted a glandular system accompanying hair, bulbus pili, hair-root and hair-root in a femoral region of the mammalian, and the said method is the following processes:

A) A process to exfoliate a lower abdominal wall status pulse skin incision is added to the said femoral region of the said mammalian, and to diverge from a thigh status pulse and a side-by-side travel nerve from a surrounding tissue

B) A process to manufacture the caulescent flap which abdominal regions subcutaneous fatty tissue of the said mammalian is separated to the size of the container mainly on the furcation of the lower abdominal wall status pulse in total, and assumes a lower abdominal wall status pulse a blood vessel handle

C) A process the said caulescent tissue piece is inserted into the said container, and to add cell included in the hair-root and NELL-1 (about 5 μg/ml-approximately 50 μg/mL)

D) A process to transplant the said container between the lines of the said femoral region in the conditions where the blood flow in the said caulescent tissue piece is not obstructed

E) A method it is a process to maintain the said container within the said line in the state that the blood flow of the said caulescent tissue piece is not obstructed for approximately two weeks or more, and to include a process to add NELL-1 (about 5 μg/ml-approximately 50 μg/mL) every other week in the said caulescent tissue piece between the said retention.

(Process C9)

The method as claimed in the item including NELL-1 is concentration of 50 μg/1 ml, and being added 20 μg/mL in total by 0.1 mL every other week for four weeks.

(Item C10)

It is a method to produce in the muscular tissue which transplanted a glandular system accompanying hair, bulbus pili, hair-root and hair-root in a femoral region of the mammalian, and the said method is the following processes:

A) A process to exfoliate a lower abdominal wall status pulse skin incision is added to the said femoral region of the said mammalian, and to diverge from a thigh status pulse and a side-by-side travel nerve from a surrounding tissue

B) A process to manufacture the caulescent flap which it is separated to the size of the container in total led by a vascular branch maintaining muscular tissue of the said mammalian, and assumes the said blood vessel a handle

C) A process to locate the collagen sponge which incorporated NELL-1 (about 5 μg/ml-approximately 50 μg/mL) or atelocollagen gel to the said container

D) A process to place a cell included in the hair-root on the said collagen sponge or atelocollagen gel

E) The process which inserts the said caulescent tissue piece into the said container

F) A process to transplant the said container between the lines of the said femoral region in the conditions where the blood flow in the said caulescent tissue piece is not obstructed

G) A method to include a process to maintain the said container within the said line in the state that the blood flow of the said caulescent tissue piece is not obstructed for approximately two weeks or more.

(Item C11)

It is a method to produce in the muscular tissue which transplanted a glandular system accompanying hair, bulbus pili, hair-root and hair-root in a femoral region of the mammalian, and the said method is the following processes:

A) A process to exfoliate a lower abdominal wall status pulse skin incision is added to the said femoral region of the said mammalian, and to diverge from a thigh status pulse and a side-by-side travel nerve from a surrounding tissue

B) A process to manufacture the caulescent flap which it is separated to the size of the container in total led by a vascular branch maintaining muscular tissue of the said mammalian, and assumes the said blood vessel a handle

C) A process the said caulescent tissue piece is inserted into the said container, and to add cell included in the hair-root and NELL-1 (about 5 μg/ml-approximately 50 μg/mL)

D) A process to transplant the said container between the lines of the said femoral region in the conditions where the blood flow in the said caulescent tissue piece is not obstructed

E) A method it is a process to maintain the said container within the said line in the state that the blood flow of the said caulescent tissue piece is not obstructed for approximately two weeks or more, and to include a process to add NELL-1 (about 5 μg/ml-approximately 50 μg/mL) regularly in the said caulescent tissue piece between the said retention.

(Item C12)

The period that is enough so that it is a method to produce in the adipose tissue which transplanted a glandular system accompanying hair, bulbus pili, hair-root and hair-root in a femoral region of the mammalian, and the above A) process adds skin incision to the said femoral region of the said mammalian, and it is a process to exfoliate from a surrounding tissue, and the above B) process can add abdominal regions subcutaneous fatty tissue of the said mammalian and the size of the container mainly on the furcation of the lower abdominal wall status pulse, and a lower abdominal wall status pulse to diverge from a femoris status pulse and a side-by-side travel nerve are separated, and it is a process to manufacture the caulescent flap which assumes a lower abdominal wall status pulse a blood vessel handle, and above NELL-1 is about 5 μg/ml-approximately 50 μg/mL, and the said sustained release anchorage is collagen sponge or an atelocollagen gel, and above F) process is a process to transplant the said container between the lines of the said femoral region in the state that the blood flow in the said caulescent tissue piece is not obstructed and an appendant glandular system is produced in above G) process in above C) process by above hair, bulbus pili, hair-root and hair-root is the method as claimed in the above item for at least approximately two Week.

(Item C13)

The period that is enough for the size of the container mainly on the vascular furcation that it is a method to produce in the muscular tissue which transplanted a glandular system accompanying hair, bulbus pili, hair-root and hair-root in a femoral region of the mammalian, and above A) process adds skin incision to the said femoral region of the said mammalian, and it is a process to exfoliate a lower abdominal wall status pulse to diverge from a femoris status pulse and a side-by-side travel nerve from a surrounding tissue, and above B) process maintains muscular tissue of the said mammalian so that it is separated in total, and it is a process to manufacture the caulescent flap which assumes the said blood vessel a handle, and above NELL-1 is about 5 μg/ml-approximately 50 μg/mL, and the said sustained release anchorage is collagen sponge or an atelocollagen gel, and above F) process is a process to transplant the said container between the lines of the said femoral region in the state that the blood flow in the said caulescent tissue piece is not obstructed, and an appendant glandular system is produced in above G) process in above C) process by above hair, bulbus pili, hair-root and hair-root is the method as claimed in the above item for at least approximately two Week.

(Item C14)

The period that is enough so that it is a method to produce in the adipose tissue which transplanted a glandular system accompanying hair, bulbus pili, hair-root and hair-root in a femoral region of the mammalian, and above A) process adds skin incision to the said femoral region of the said mammalian, and process above B) process to exfoliate from a surrounding tissue can add abdominal regions subcutaneous fatty tissue of the said mammalian and the size of the container mainly on the furcation of the lower abdominal wall status pulse, and a lower abdominal wall status pulse to diverge from a femoris status pulse and a side-by-side travel nerve are separated, and it is a process to manufacture the caulescent flap which assumes a lower abdominal wall status pulse a blood vessel handle, and above NELL-1 is about 5 μg/ml-approximately 50 μg/mL, and above D) process is a process to transplant the said container between the lines of the said femoral region in the state that the blood flow in the said caulescent tissue piece is not obstructed, and an appendant glandular system is produced in above E) process in above C) process by above hair, bulbus pili, hair-root and hair-root is the method as claimed in the above item which NELL-1 is about 5 μg/ml-approximately 50 μg/mL, and is added between the said retention every other week by the said caulescent tissue piece for at least approximately two Week.

(Item C15)

The period that is enough for the size of the container mainly on the vascular furcation that it is a method to produce in the muscular tissue which transplanted a glandular system accompanying hair, bulbus pili, hair-root and hair-root in a femoral region of the mammalian, and above A) adds skin incision to the said femoral region of the said mammalian, and it is a process to exfoliate a lower abdominal wall status pulse to diverge from a femoris status pulse and a side-by-side travel nerve from a surrounding tissue, and above B) maintains the muscular tissue of the said albino rat so that it is separated in total, and it is a process to manufacture the caulescent flap which assumes the said blood vessel a handle, and above NELL-1 is about 5 μg/ml-approximately 50 μg/mL, and above D) process is a process to transplant the said container between the lines of the said femoral region in the state that the blood flow in the said caulescent tissue piece is not obstructed, and an appendant glandular system is produced in above E) process in above C) process by above hair, bulbus pili, hair-root and hair-root is the method as claimed in the above item which NELL-1 is about 5 μg/ml-approximately 50 μg/mL, and is added between the said retention every other week by the said caulescent tissue piece for at least approximately two Week.

(Item D1)

It is a method to maintain in the adipose tissue which transplanted a liver tissue piece in an organic femoral region or muscular tissue, and the said method is the following processes:

A) A process to manufacture a space to port a container in the said organism

B) A process the said organic adipose tissue or muscular tissue is separated to the size of the container with a blood vessel maintaining the said adipose tissue or muscular tissue in total, and to manufacture the caulescent flap including the blood vessel handle

C) A process to locate the controlled-release false work which incorporated NELL-1 to the said container,

D) A process to place the said liver tissue piece on the said controlled-release anchorage

E) The process which inserts the said caulescent tissue piece into the said container

F) A process to transplant the said container in the state that the blood flow in the said caulescent tissue piece is not obstructed in the said space and

G) A method to include a process to put in the state that the blood flow of the said caulescent tissue piece is not obstructed.

(Item D2)

It is a method to maintain in the adipose tissue which transplanted a liver tissue piece in an organic femoral region or muscular tissue, and the said method is the following processes:

A) A process to manufacture a space to port a container in the said organism

B) A process the said organic adipose tissue or muscular tissue is separated to the size of the container with a blood vessel maintaining the said adipose tissue or muscular tissue in total, and to manufacture the caulescent flap including the blood vessel handle

C) A process the said caulescent tissue piece is inserted into the said container, and to add said liver tissue piece and NELL-1

D) A process to transplant the said container in the said space in the conditions where the blood flow in the said caulescent tissue piece is not obstructed

E) A method to include a process to put in the state that the blood flow of the said caulescent tissue piece is not obstructed.

(Item D3)

The method as claimed in the above item which is the size that the container is in the space.

(Item D4)

The method as claimed in the item where the space is manufactured between the lines in the femoral region.

(Item D5)

The method as claimed in the item where NELL-1 is contained with about 5 μg/ml-approximately 50 μg/mL.

(Item D6)

The controlled-release anchorage is the method as claimed in collagen sponge and an above item selected than the cluster comprising atelocollagen gels.

(Item D7),

It is a method to maintain in the adipose tissue which transplanted a liver tissue piece in a femoral region of the mammalian, and the said method is the following processes:

A) A process to exfoliate a lower abdominal wall status pulse skin incision is added to the said femoral region of the said mammalian, and to diverge from a thigh status pulse and a side-by-side travel nerve from a surrounding tissue

B) A process to manufacture the caulescent flap which abdominal regions subcutaneous fatty tissue of the said mammalian is separated to the size of the container mainly on the furcation of the lower abdominal wall status pulse in total, and assumes a lower abdominal wall status pulse a blood vessel handle

C) A process to place the collagen sponge which incorporated NELL-1 (about 5 μg/ml-approximately 50 μg/mL) into the said container or atelocollagen gel,

D) A process to place the said liver tissue piece on the said collagen sponge or atelocollagen gel

E) The process which inserts a caulescent tissue piece into the said container

F) A process to transplant the said container between the lines of the said femoral region in the conditions where the blood flow in the said caulescent tissue piece is not obstructed, and

G) A method to include a process to put the said container in the state that the blood flow of the said caulescent tissue piece is not obstructed.

(Item D8)

It is a method to maintain in the adipose tissue which transplanted a liver tissue piece in a femoral region of the mammalian, and the said method is the following processes:

A) A process to exfoliate a lower abdominal wall status pulse skin incision is added to the said femoral region of the said mammalian, and to diverge from a thigh status pulse and a side-by-side travel nerve from a surrounding tissue

B) A process to manufacture the caulescent flap which abdominal regions subcutaneous fatty tissue of the said mammalian is separated to the size of the container mainly on the furcation of the lower abdominal wall status pulse in total, and assumes a lower abdominal wall status pulse a blood vessel handle

C) A process the said caulescent tissue piece is inserted into the said container, and to add said liver tissue piece and NELL-1 (about 5 μg/ml-approximately 50 μg/mL)

D) A process to transplant the said container between the lines of the said femoral region in the conditions where the blood flow in the said caulescent tissue piece is not obstructed

E) A method it is a process to put the said container in the state that the blood flow of the said caulescent tissue piece is not obstructed, and, meanwhile, to include a process to add NELL-1 (about 5 μg/ml-approximately 50 μg/mL) every other week in the said caulescent tissue piece.

(Process C9)

The method as claimed in the item including NELL-1 is concentration of 50 μg/1 ml, and being added 20 μg/mL in total by 0.1 mL every other week for four weeks.

(Item D10)

It is a method to maintain in the muscular tissue which transplanted a liver tissue piece in a femoral region of the mammalian, and the said method is the following processes:

A) A process to exfoliate a lower abdominal wall status pulse skin incision is added to the said femoral region of the said mammalian, and to diverge from a thigh status pulse and a side-by-side travel nerve from a surrounding tissue

B) A process to manufacture the caulescent flap which it is separated to the size of the container in total led by a vascular branch maintaining muscular tissue of the said mammalian, and assumes the said blood vessel a handle

C) A process to locate the collagen sponge which incorporated NELL-1 (about 5 μg/ml-approximately 50 μg/mL) or atelocollagen gel to the said container

D) A process to place the said liver tissue piece on the said collagen sponge or atelocollagen gel

E) The process which inserts the said caulescent tissue piece into the said container

F) A process to transplant the said container between the lines of the said femoral region in the conditions where the blood flow in the said caulescent tissue piece is not obstructed

G) A method to include a process to put the said container in the state that the blood flow of the said caulescent tissue piece is not obstructed.

(Item D11)

It is a method to maintain in the muscular tissue which transplanted a liver tissue piece in a femoral region of the mammalian, and the said method is the following processes:

A) A process to exfoliate a lower abdominal wall status pulse skin incision is added to the said femoral region of the said mammalian, and to diverge from a thigh status pulse and a side-by-side travel nerve from a surrounding tissue

B) A process to manufacture the caulescent flap which it is separated to the size of the container in total led by a vascular branch maintaining muscular tissue of the said mammalian, and assumes the said blood vessel a handle

C) A process the said caulescent tissue piece is inserted into the said container, and to add said liver tissue piece and NELL-1 (about 5 μg/ml-approximately 50 μg/mL)

D) A process to transplant the said container between the lines of the said femoral region in the conditions where the blood flow in the said caulescent tissue piece is not obstructed

E) A method it is a process to put the said container in the state that the blood flow of the said caulescent tissue piece is not obstructed, and, meanwhile, to include a process to add NELL-1 (about 5 μg/ml-approximately 50 μg/mL) regularly in the said caulescent tissue piece.

(Item D12)

The method as claimed in the above item which it is a method to maintain in the adipose tissue which transplanted a liver tissue piece in a femoral region of the mammalian, and the above A) process adds skin incision to the said femoral region of the said mammalian, and the process above B) process to exfoliate a lower abdominal wall status pulse to diverge from a femoris status pulse and a side-by-side travel nerve from a surrounding tissue separates abdominal regions subcutaneous fatty tissue of the said mammalian to the size of the container mainly on the furcation of the lower abdominal wall status pulse in total, and it is a process to manufacture the caulescent flap which assumes a lower abdominal wall status pulse a blood vessel handle, and is a process to transplant the said container between the lines of the said femoral region in the state that above NELL-1 is about 5 μg/ml-approximately 50 μg/mL, and the said sustained release anchorage is collagen sponge or an atelocollagen gel and the blood flow in the said caulescent tissue piece is not obstructed as for the above F) process in above C) process.

(Item D13)

The method as claimed in the above item which it is a method to maintain in the muscular tissue which transplanted a liver tissue piece in a femoral region of the mammalian, and the above A) process adds skin incision to the said femoral region of the said mammalian, and the process above B) process to exfoliate from a surrounding tissue separates a lower abdominal wall status pulse to diverge from a femoris status pulse and a side-by-side travel nerve to the size of the container mainly on vascular furcation maintaining muscular tissue of the said mammalian in total, and it is a process to manufacture the caulescent flap which assumes the said blood vessel a handle, and is a process to transplant the said container between the lines of the said femoral region in the state that above NELL-1 is about 5 μg/ml-approximately 50 μg/mL, and the said sustained release anchorage is collagen sponge or an atelocollagen gel and the blood flow in the said caulescent tissue piece is not obstructed as for the above F) process in above C) process.

(Item D14)

It is a method to maintain in the adipose tissue which transplanted a liver tissue piece in a femoral region of the mammalian, and the said method is the following processes: The method as claimed in the above item which skin incision is added to the said femoral region of the said mammalian, and the process above B) process to exfoliate a lower abdominal wall status pulse to diverge from a femoris status pulse and a side-by-side travel nerve from a surrounding tissue separates abdominal regions subcutaneous fatty tissue of the said mammalian to the size of the container mainly on the furcation of the lower abdominal wall status pulse in total, and the A) process is a process to manufacture the caulescent flap which assumes a lower abdominal wall status pulse a blood vessel handle, and NELL-1 is about 5 μg/ml-approximately 50 μg/mL, and above NELL-1 is about 5 μg/ml-approximately 50 μg/mL, and above D) process is a process to transplant the said container between the lines of the said femoral region in the state that the blood flow in the said caulescent tissue piece is not obstructed and is added in above E) process in above C) process every other week by an above caulescent tissue piece.

(Item D15)

It is a method to maintain in the muscular tissue which transplanted a liver tissue piece in a rat femoral region, and the said method is the following processes: The method as claimed in the above item which skin incision is added to the said femoral region of the said mammalian, and the process above B) process to exfoliate from a surrounding tissue separates a lower abdominal wall status pulse to diverge from a thigh status pulse and a side-by-side travel nerve to the size of the container mainly on vascular furcation maintaining muscular tissue of the said mammalian in total, and the A) process is a process to manufacture the caulescent flap which assumes the said blood vessel a handle, and NELL-1 is about 5 μg/ml-approximately 50 μg/mL, and above NELL-1 is about 5 μg/ml-approximately 50 μg/mL, and above D) process is a process to transplant the said container between the lines of the said femoral region in the state that the blood flow in the said caulescent tissue piece is not obstructed and is added in above E) process in above C) process every other week by an above caulescent tissue piece.

Thus, a thing becoming obvious to those skilled in the art is understood if these of the present invention and the other advantages read the following detailed description when taken with the drawing and it is understood.

### Effect of the Invention

In accordance with the invention, a technique using NELL-1 to form a differentiation induced cell, tissue and an organ is provided. Particularly, the technique of the present invention can form a differentiation induced cell, tissue and an organ in allopatry. A differentiation induced cell, tissue and the organ have prominent effect outside expectation to be able to run the function that they originally have by the present invention.

The formation technology of a cell by such NELL-1, tissue and the organ can be applied to the cell grafting treatment using the undifferentiated cells such as recent embryonic stem cells. In other words desired differentiation is destined to the undifferentiated cells such as embryonic stem cells, and the organ which functions in vivo by further triggering NELL-1 can be formed. Also, to the patient who cannot enjoy organ transplantation and a highly medical benefit by the thing for an autochthonous cell, tissue and organs, a substitute organ can be provided.

A technique using NELL-1 to maintain a cell, tissue transplanted in an organism by the present invention and an organ in another situation is provided. According to the technique of the present invention, transplanted cell, tissue and the organ maintain form and size, and it has advantageous effect outside expectation to be able to maintain the function that they further originally have.

Such an effect is provided only after it depends on the present invention.

### BRIEF DESCRIPTION OF DRAWINGS

[FIG. 1A]

FIG. 1A shows the sketch of the human NELL1 plasmid construction.

[FIG. 1B]

FIG. 1B is a continuance of FIG. 1A.

[FIG. 1C]

FIG. 1C is a continuance of FIG. 1B.

[FIG. 1D]

FIG. 1 shows the adipose tissue in the silicon mold and hematopoietic system tissue formed in the environment with a blood vessel maintaining it. The bar of photographs is 2.0 mm.

[FIG. 2]

FIG. 2 shows a glandular system formed in the environment with the adipose tissue in the silicon mold and a blood vessel maintaining it. The bar of photographs is 200 μm. G: The colloidal secreted material which was secreted by epithelial cells (single-layered columnar epithelia). P: The pipe which exhausts colloidal secreted material surrounded by single-layered cuboidal epithelia

[FIG. 3]

FIG. 3 shows a glandular system formed in the environment with the adipose tissue in the silicon mold and a blood vessel maintaining it. The bar of photographs is 200 μm. G: Granular secreted material exists among epithelial cells (single-layered columnar epithelia) inside.

[FIG. 4]

The liver tissue piece which added a physiological salt solution is transplanted, and FIG. 4 shows the state of the grafting part four weeks after the grafting in the environment with adipose tissue and a blood vessel maintaining it.

[FIG. 5]

The liver tissue piece which added NELL-1 (5 μg) is transplanted, and FIG. 5 shows the state of the grafting part four weeks after the grafting in the environment with adipose tissue and a blood vessel maintaining it. A part surrounded with a broken line is the hepatic tissue piece which transplanted. The bar shows 1.0 mm.

[FIG. 6A]

The incubator in the organism which added only FGF is ported, and FIG. 6A shows the histology of the grafting part two weeks after the grafting in the environment with adipose tissue and a blood vessel maintaining it. The bar shows 250 μmm.

[FIG. 6B]

FIG. 6B is an enlarged view of FIG. 6A. The bar shows 500 μmm.

A: Connective tissue,

B: A blood vessel

[FIG. 6C]

The incubator in the organism which added NELL-1 and FGF is ported, and FIG. 6C shows the histology of the grafting part two weeks after the grafting in the environment with adipose tissue and a blood vessel maintaining it.

[FIG. 6D]

FIG. 6D is an enlarged view of FIG. 6C. The bar shows 500 μmm.

A: Connective tissue, B: A differentiated glandular system

[FIG. 6E]

The incubator in the organism which added only NELL-1 is ported, and FIG. 6E shows the histology of the grafting part two weeks after the grafting in the environment with adipose tissue and a blood vessel maintaining it. The bar shows 250 μmm.

[FIG. 6F].

FIG. 6F is an enlarged view of FIG. 6E.

The bar shows 500 μmm.

An arrow:A glandular system is shown

[FIG. 6G]

The incubator in the organism which added only a physiological salt solution is ported, and FIG. 6E shows the histology of the grafting part two weeks after the grafting in the environment with adipose tissue and a blood vessel maintaining it.

[FIG. 7]

FIG. 7 is the cluster which administered NELL-1 in a mold consecutively from the outside the body.

[FIG. 8]

After FIG. 8 administered NELL-1 in a mold consecutively from the outside the body, it is the fructification that examined validity of this NELL-1 dosage by an injected thing from the outside the body with stupid toxin dilution. As a result, it was confirmed that the dosage of NELL-1 was certain. The point that injected hematoxylin dilution was dyed by purple.

[FIG. 9]

FIG. 9 is a photograph of the hematoxylin eosin staining which shows a glandular system appendant in hair, bulbus pili, hair-root formed in the environment with adipose tissue and a blood vessel maintaining it and hair-root.

The bar shows 500 μm.

A: The glandular system which accompanies hair-root

B: Hair and the root of hair (bulbus

### SEQUENCE LISTING DESCRIPTION

SEQ ID NO: 1:

It is a nucleotide sequence of human NELL1. The region where the region encoding signal peptide encodes base 135-182, maturation peptide is base 183-2564.

SEQ ID NO: 2:

It is amino acid sequencing of human NELL1. As for the signal peptide, residue 1-16, maturation peptide are residue 17-810.

SEQ ID NO: 3: It is a nucleotide sequence of mouse NELL1. The region where the region encoding signal peptide encodes base 40-102, maturation peptide is base 103-2472.

SEQ ID NO: 4: It is amino acid sequencing of mouse NELL1. As for the signal peptide, residue 1-21, maturation peptide are residue 22-810.

SEQ ID NO: 5: It is a nucleotide sequence of rat NELL1. The region where the region encoding signal peptide encodes base 59-121, maturation peptide is base 122-2491.

SEQ ID NO: 6: It is amino acid sequencing of rat NELL1. As for the signal peptide, residue 1-21, maturation peptide are residue 22-810.

SEQ ID NO: 7: It is vectorial sequence used in an embodiment.

SEQ ID NO: 8: It is the nucleotide sequence of V5 tag and the histidine tag (a His tag).

SEQ ID NO: 9: It is the amino acid sequencing of V5 tag and the histidine tag (a His tag).

SEQ ID NO: 10:

It is amino acid sequencing of the original signal peptide of HumanNELL1.

SEQ ID NO: 11:

It is amino acid sequencing of the signal peptide of HoneyBeeMellitin.

SEQ ID NO: 12:

It is a nucleotide sequence of Xenopus laevis NELL1.

SEQ ID NO: 13:

It is amino acid sequencing of Xenopus laevis NELL1.

SEQ ID NO: 14:

It is the amino acid sequencing of the hHELL1 fragment.

SEQ ID NO: 15:

It is the amino acid sequencing of the hLAMA3 fragment.

SEQ ID NO: 16:

It is the amino acid sequencing of the mLAMA3 fragment.

SEQ ID NO: 17:

It is the amino acid sequencing of the hCOLA1 fragment.

SEQ ID NO: 18:

It is the amino acid sequencing of the hTSP1 fragment.

SEQ ID NO: 19:

It is sequence of forward primer #2.

SEQ ID NO: 20:

It is sequence of forward primer #3.

SEQ ID NO: 21:

It is sequence of forward primer #4.

SEQ ID NO: 22:

It is sequence of forward primer #5.

SEQ ID NO: 23:

It is sequence of reverse primer #2.

SEQ ID NO: 24:

It is sequence of reverse primer #3.

SEQ ID NO: 25:

It is sequence of reverse primer #4.

SEQ ID NO: 26:

It is sequence of reverse primer #5.

SEQ ID NO: 27:

It is the sequence of the V5 tag.

SEQ ID NO: 28:

It is the sequence of the histidine tag.

SEQ ID NO: 29:

It is the sequence of the tag portion used in embodiment.

SEQ ID NO: 30:

It is sequence of forward primer #1.

SEQ ID NO: 31:

It is sequence of reverse primer #1.

### MODE TO CARRY OUT INVENTION

The present invention is described as follows. Over the whole of the present specification, it should be understood that the expression of the singular form includes the general idea of the plural form unless otherwise specified. Thus, it should be understood that the article (when, e.g., it is English “a”, “an”, “the”) of the singular form includes the general idea of the plural form unless otherwise specified. Also, the term as used herein is the field of this above unless otherwise specified, and what is used in a commonly used meaning should be understood. Thus, all technical terms as used herein and technology term have a meaning same as what is understood by those skilled in the art of this field of the invention for the public unless it is defined elsewhere. When it is contradicted, the present specification (and including a definition) takes first priority.

(The Definition of the Term)

The definitions of the term which is used below in the present specification in particular are enumerated.

Herein, “NELL-1” is a gene found by Ting, K. et al., and a thing identified as an etiology gene of the craniosynostosis as NELL-1 gene is a beginning (patent document 1-4). And craniosynostosis of phenotype was ensured when the mouse which then overexpressed NELL-1 gene that these inventors came to discover NELL-1 as the protein using the YeastTwoHybrid method (Kuroda, S., Oyasu, M., Kawakami, M., Kanayama, N., Tanizawa, K., Saito, N., Abe, T-head. Matsuhashi, S. and Ting, 1999 K. ( ) Biochem. Biophys. Res. Commun. 265, 79-86) was manufactured. Also, when NELL-1 gene is introduced into mouse outrider osteoblasts MC3T3-E1 using Adenoviridae, active elevation of the alkaline phosphatase in the cell which is a marker is ensured for a bone differentiation early stage, and it is known that Osteopontin which is a marker, an expression of the mRNA of Osteocalsin rising, the cell which NELL-1 gene was finally introduced into caused a calcareous deposit more in differentiated cells for metaphase/anaphase (Zhang, X., Kuroda, S., Carpenter, D., Nishimura, I., Soo, C., Moats, R., Iida, K., Wisner, E., Hu, F. Y., Miao, S., Beanes, S., Dang, C., Vastardis, H., Longaker, M., Tanizawa, K., Kanayama, N., Saito, N. and Ting, 2002 K. ( ) J. Clin. Invest. 18, 2126-2134). Herein, it can trade with “NELL-1” and “NELL1”, and it can be used.

Herein, “the material that it is changed to function as NELL-1” means the material which can produce NELL-1. For example, the material which can produce NELL-1 using a genetic engineering technology such as plasmid, the viral vector can be used in constituents of the present invention.

It is understood to make things of the maturation sequence usually done at time called “NELL-1” or “NELL1” in the present specification. Thus, with the present invention, it should usually pay attention to not including a signal sequence (a leader sequence) at time called “NELL-1” or “NELL1”. The nucleotide sequence of NELL1 is represented as follows specifically.

(1) Amino acid sequencing shown in SEQ ID NO: 2, 4, 6 or 13 (amino acid sequencing of NELL1),

(2) In amino acid sequencing shown in SEQ ID NO: 2, 4, 6 or 13 (amino acid sequencing of NELL1), 1 or several substitutions, addition and/or a deficiency are included, and amino acid sequencing showing the activity of nature model NELL1, And

(3) The homology of amino acid sequencing and at least 70% shown in SEQ ID NO: 2, 4, 6 or 13 (amino acid sequencing of NELL1) amino acid sequencing, And

(4) Amino acid sequencing encoded by a nucleotide sequence shown in SEQ ID NO: 1, 3, 5 or 12 (a nucleotide sequence encoding NELL1) or a nucleotide sequence to hybridize under a complementary nucleotide sequence and a stringent condition to miss,

(5) Amino acid sequencing encoded by a nucleotide sequence shown in SEQ ID NO: 1, 3, 5 or 12 (a nucleotide sequence encoding NELL1) or a nucleotide sequence to hybridize under a complementary nucleotide sequence and a moderate stringent condition to miss,

(6) Amino acid sequencing encoded by a nucleotide sequence having at least 70% of homologies in a nucleotide sequence shown in SEQ ID NO: 1, 3, 5 or 12 (a nucleotide sequence encoding NELL1),

(7) (1) A homolog of . . . (6) or an allelic variant can be raised.

Nucleic acid encoding NELL1 or the NELL gene can be represented as follows.

(1) A nucleotide sequence encoding amino acid sequencing shown in SEQ ID NO: 2, 4, 6 or 13 (amino acid sequencing of NELL1),

(2) The nucleotide sequence which 1 or several substitutions, addition and/or a deficiency are included in amino acid sequencing shown in SEQ ID NO: 2, 4, 6 or 13 (amino acid sequencing of NELL1) and encodes amino acid sequencing showing the activity of nature model NELL1 and

(3) A nucleotide sequence encoding amino acid sequencing with amino acid sequencing shown in SEQ ID NO: 2, 4, 6 or 13 (amino acid sequencing of NELL1) and at least 70% of homologies and

(4) A nucleotide sequence shown in SEQ ID NO: 1, 3, 5 or 12 (a nucleotide sequence encoding NELL1) or a nucleotide sequence to hybridize under a complementary nucleotide sequence and a stringent condition to miss,

(5) A nucleotide sequence shown in SEQ ID NO: 1, 3, 5 or 12 (a nucleotide sequence encoding NELL1) or a nucleotide sequence to hybridize under a complementary nucleotide sequence and a moderate stringent condition to miss,

(6) A nucleotide sequence having at least 70% of homologies in a nucleotide sequence shown in SEQ ID NO: 1, 3, 5 or 12 (a nucleotide sequence encoding NELL1),

(7) (1) A homolog of . . . (6) or an allelic variant can be raised.

Here, in the SEQ ID NO:, SEQ ID NO: 1 is a nucleotide sequence of human NELL1. The region where the region encoding signal peptide encodes base 135-182, maturation peptide is base 183-2564, and SEQ ID NO: 2 is amino acid sequencing of human NELL1. As for the signal peptide, residue 1-16, maturation peptide are residue 17-810.

SEQ ID NO: 3 is a nucleotide sequence of mouse NELL1. The region where the region encoding signal peptide encodes base 40-102, maturation peptide is base 103-2472, and SEQ ID NO: 4 is amino acid sequencing of mouse NELL1. As for the signal peptide, residue 1-21, maturation peptide are residue 22-810.

SEQ ID NO: 5 is a nucleotide sequence of rat NELL1. The region where the region encoding signal peptide encodes base 59-121, maturation peptide is base 122-2491, and SEQ ID NO: 6 is amino acid sequencing of rat NELL1. As for the signal peptide, residue 1-21, maturation peptide are residue 22-810.

Thus, in the present invention, what can be used in the present invention based on information of the signal sequence and the maturation sequence is understood by those skilled in the art.

About SEQ ID NO: 12 and 13, it is partial sequence, but those skilled in the art arrange above information in a signal sequence and maturation for the cause (the nucleic acid sequence which, i.e., encodes polypeptide comprising the applicable amino acids of the maturation portion or it), and it is identified, and it is understood that it can be used in the present invention.

Herein, “protein”, “polypeptide”, “oligopeptide” and “the peptide” are used in the same meaning in the present specification, and it refers to the polymer of the amino acid of the length at will. Even if this polymer is linear chain, it may diverge and may be annular. Even if the amino acid is natural, it may be non-natural, and it may be a modified amino acid. This term can include the thing which was also assembled to a complex of a plurality of polypeptide chain. This term also includes a nature or the amino acid polymer modified artificially. For example, as such a modification, a disulphide bonding, glycosylation, a lipidation are acetylated, phosphorylation or at will other engineers or a modification (the aggregate with the, e.g., labeled ingredient). For example, this definition also includes the polypeptide (including the amino acids which, e.g., are non-natural) including the analogs 1 or 2 or more of the amino acid, a peptide of compound (e.g., peptoid) and other modifications well known in the art.

Herein, as far as “the amino acid” satisfies object of the invention, even a non-natural thing is preferable with the natural thing.

Herein, it is different from “an amino acid inductor” or “the amino acid analog” with the existing amino acid naturally, but it refers to a thing having the function like the original amino acid. Such an amino acid inductor and the amino acid analog are well known in the art. As far as, in the present specification, an amino acid inductor and the amino acid analog can provide a biological function same as an amino acid, it is understood that it can be used as substitution.

Herein, “the natural amino acid” means the L-isomer of a natural amino acid. The natural amino acid is glycine, alanine, a 2-amino-3-methylbutyric acid, leucine, isoleucine, serine, methionine, threonine, phenylalanine, tyrosine, tryptophan, Cys, proline, histidine, an asparaginic acid, asparagine, glutamate, glutamine, y-carboxy glutamate, arginine, ornithine and lysin. All amino acids to refer to in the present specification are L bodies unless it is shown in particular, but there is also the form using the amino acid of the D body within the present invention, too.

Herein, “non-naturally occurring amino acid” usually means the amino acid which is not found in protein naturally. An example of the non-naturally occurring amino acid includes norleucine, para-nitrophenylalanine, homophenylalanine, para-fluorophenylalanine, 3-amino-2-benzyl propionate, D body of the homoarginine or L body and D-phenylalanine.

Herein, “the amino acid analog” is not an amino acid, but it refers to a monad similar to the properties of matter of the amino acid and/or a function. For example, an amino acid analog includes ethionine, canavanine, 2-methyl glutamine. With the amino acid mimicker, a conformation unlike the common structure of the amino acid is provided, but it refers to a compound functioning in the form which is similar to a naturally existing amino acid.

Herein, it is interchangeable with a gene, cDNA, mRNA, oligonucleotide and polynucleotide, and “the nucleic acid” is also used. The particular nucleotide sequence also includes “a splice modified body”. The particular protein encoded by a nucleic acid includes arbitrary protein encoded by the splice modification body of the nucleic acid tacitly likewise. “The splice modified body” is a product of the genetic alternative splice so that the name suggests. After transfer, the first nucleic acid transcript can be spliced so that a different nucleic acid splice product encodes different polypeptide. The splice modified corporeal yield mechanism changes, but exonic alternative splice is included. This definition also includes another polypeptide coming from the same nucleic acid by a read-through transcription. This definition includes any product (including the splice product which is in recombination form) of the splice reaction. Alternatively, the allelic variant is in this range, too.

Herein, “polynucleotide”, “oligonucleotide” and “the nucleic acid” are used in the same meaning in the present specification, and it refers to a nucleotidic polymer of the length at will. This term also includes “an oligonucleotide inductor” or “a polynucleotide inductor”. With “an oligonucleotide inductor” or “the polynucleotide inductor”, a nucleotidic inductor is included, or symphysis of the internucleotide refers to different oligonucleotide or polynucleotide with the common, and it is used interchangeably. For example, 2′-O-methyl-ribonucleotide, the oligonucleotide inductor that oligonucleotide phosphodiester bonds were converted into phosphorothioate symphysis, the oligonucleotide inductor that oligonucleotide phosphodiester bonds were converted into N3′-P5′ phosphoro net date symphysis, the oligonucleotide inductor that oligonucleotide ribose and phosphodiester bonds were converted into peptide nucleic acid symphysis, oligonucleotide uracil were C-5 propynyl uracil, and a substituted oligonucleotide inductor, oligonucleotide uracil were C-5 thiazole uracil, and a substituted oligonucleotide inductor, oligonucleotide cytosine were C-5 propynyl cytosine, and a substituted oligonucleotide inductor, the oligonucleotide inductor that oligonucleotide cytosine was substituted for phenoxazine modification cytosine (phenoxazine-modifiedcytosine), DNA ribose were 2′-O-propyl ribose, and substituted oligonucleotide inductor and oligonucleotide ribose was substituted for 2′-methoxyethoxy ribose as such an oligonucleotide specifically Oligonucleotide inductors are exemplified. Besides, that the particular nucleotide sequence includes the modification body (e.g., a degeneracy codon substitution product) modified conservatively and complementary sequence like the sequence that was also shown explicitly is planned if not shown when not so. Specifically, the degeneracy codon substitution product can be accomplished because the third locus of 1 or the further selected (or all) codon makes sequence substituted for a shuffling base and/or a deoxy lno residue (Batzer et al., Nucleic Acid Res. 19: 5081 (1991), Ohtsuka et al., J. Biol. Chem. 260: 2605-2608 (1985), Rossolini et al., Mol. Cell. Probes 8: 91-98 (1994)).

Herein, “the nucleotide” may be the non-natural thing with the natural thing. Nucleotidic inductor or “nucleotidic analog” refers to the thing which it is different from the existing nucleotide naturally, but has a function like the original nucleotide. Such a nucleotidic inductor and the nucleotidic analog are well known in the art. As such a nucleotidic inductor and a nucleotidic analog example, phosphorothioate, phosphoramidate, methyl phosphonate, chiral methyl phosphonate, 2′-O-methyl ribonucleotide, a peptide model nucleic acid (PNA) are included, but is not limited to these.

Herein, with “the search”, it refers to finding other nucleic acid base sequence to have a particular function and/or character using for an electron or the biological nucleic acid base sequence that alternatively there is by other methods. An electronic search includes BLAST (Altschuletal., J. Mol. Biol. 215: 403-410 (1990)), FASTA (Pearson & Lipman, Proc. Natl. Acad. Sci., USA 85: 2444-2448 (1988)), the Smith and Waterman method (Smith and Waterman, J. Mol. Biol. 147: 195-197 (1981)) and the Needleman and Wunsch method (Needleman and Wunsch, J. Mol. Biol. 48: 443-453 (1970)), but is not limited to them. A biological search includes microarrays (microarrays assay), the PCR which were able to stick to stringent hybridization, the macro array which stuck genomic DNA on nylon membranes or glass plate and insitu hybridization, but is not limited to them. Herein, to a gene as used herein, it is intended to include the correspondence gene identified by such an electronic search, a biological search.

Herein, “the stringent condition” for hybridization means the condition that a complementary strand of the nucleotide chain which a complementary strand of nucleotide chain having a similarity or a homology to a target sequence hybridizes in a target sequence with precedence and does not have a similarity or a homology does not hybridize substantially. With “the complementary strand” of a certain nucleotide sequence, it refers to an anti-nucleotide sequence (e.g., T-head to A, C to G) to unite based on the hydrogen bond between the base of the nucleic acid. A stringent condition is a sequence dependence mark and is different in various kinds of situation. The longer sequence specifically hybridizes at higher temperature. Generally approximately 5 degrees Celsius is lower than thermal melting temperature (Tm) on the particular sequence at stated ionic strength and pH, and the stringent condition is selected. The Tm is the temperature that complementary nucleotidic 50% hybridize in a target sequence with a balance in a target sequence under stated ionic strength, pH and nucleic acid density. “A stringent condition” is a sequence dependence mark and is different by various kinds of environmental parameters. The common guideline of the hybridization of the nucleic acid is found by Tijssen (Tijssen (1993), Laboratory Technniques In Biochemistry And Molecular Biology-Hybridization With Nucleic Acid Probes Part I, Chapter 2 “Overview of principles of hybridization and the strategy of nucleic acid probe assay”, Elsevier, New York).

Salt concentration is less than about 1.0 MNa+, and the stringent condition is Na+ density of approximately 0.01-1.0M at pH 7.0-8.3 typically typically (or other salts) and temperature is at least approximately 60 degrees Celsius about at least approximately 30 degrees Celsius and the nucleotide (e.g., it is longer than 50 nucleotide) had a long about the nucleotide (e.g., 10-50 nucleotide) having a short. The stringent condition can be also accomplished by addition of the non-stabilizer such as the formamide. A stringent condition in the present specification includes irrigation at 60 degrees Celsius in 50% of form amide, NaCl of 1M, hybridization in the buffer of 1% of SDS (37 degrees Celsius) and 0.1*SSC.

Herein, with “the polynucleotide to hybridize in a stringent condition”, it refers to a conventional done well-known condition in the art. Such a polynucleotide can be obtained by using colony hybridization, the plaque hybridization method or Southern blotting hybridization as a probe in selected polynucleotide from all over the polynucleotide of the present invention. Specifically, after having performed hybridization at NaCl presence bottom of 0.7-1.0M, 65 degrees Celsius using the filter which immobilized DNA derived from a colony or plaque, SSC (saline-sodiumcitrate) solution (the composition of the SSC solution of the 1 time density is 150 mM sodium chloride, 15 mM sodium citrate) of the 0.1-2 times density is used, and the polynucleotide which can be identified by washing a filter under a 65 degrees Celsius condition is meant. Hybridization is Molecular Cloning 2nd ed., Current Protocols in Molecular Biology, Supplement 1-38, DNA Cloning 1: The method described in the experiments book such as Core Techniques, A Practical Approach, Second Edition, Oxford University Press (1995) is followed, and it can be conducted. Here, from sequence to hybridize under a stringent condition, sequence only including only A sequence or T-head sequence is preferably excluded. Polynucleotide which can be hybridized refers to the polynucleotide which another polynucleotide can hybridize under the hybridization Glycine max condition. Polynucleotide having a sequence of DNA encoding polypeptide having amino acid sequencing shown with the present invention specifically and homologies at least 60% or more preferably polynucleotide having 80% homologies or more, polynucleotide having 90% homologies or more more preferably polynucleotide having 95% homologies or more can be raised as the polynucleotide which can be hybridized specifically.

Herein, it refers to the condition that was designed to exclude hybridization of the DNA which “the highly stringent condition” enables the hybridization of a DNA chain having high complementarity in a nucleotide sequence and significantly has mismatch. The strike phosphorus Gen sea of the hybridization is important, and it is determined by a condition of the denaturant such as temperature, ionic strength and the formamide. The example of “a highly stringent condition” about such a hybridization and the irrigation is 0.0015M sodium chloride, 0.0015M sodium citrate, 65-68 degrees Celsius or 0.015M sodium chloride, 0.0015M sodium citrate and 50% formamide, 42 degrees Celsius. A Laboratory Manual, 2nd Edition, Cold Spring Harbor Laboratory (Cold Spring Harbor, N, lateral .1989) and Anderson et al., Nucleic Acid Hybridization refer to a Practical approach, IV, IRLPressLimited (Oxford, England) Limited, Oxford, England for such a highly stringent condition Sambrook et al., Molecular Cloning. By a need, a more stringent condition (the temperature that, e.g., is higher, the formamide which are higher than lower ionic strength or other denaturant) may be used. It can be included the hybridization that other medicine is non-specific and/or the hybridization of the background in a hybridization buffer solution and an irrigation buffer solution for the purpose of decreasing. For an example of such an other medicine, it is 0.1% bovine serum albumin, 0.1% polyvinylpyrrolidone, 0.1% sodium pyrophosphate, 0.1% sodiumdodecyl sulfate (NaDodSO4 or SDS), Ficoll, Denhardt solution, sonicated Oncorhynchus keta sperm DNA (or another non-complementary DNA) and dextran sulfate, but the appropriate medicine of the others can be also used. The density of these additives and the model can be changed without affecting the strike phosphorus Gen sea of the hybridization condition substantially. Hybridization experiments are usually carried out at pH 6.8-7.4, but, In a representative ionic strength condition, a rate of the hybridization is most pH independence. Anderson et al., Nucleic Acid Hybridization refer to a Practical Approach, Chapter 4, IRL Press Limited (Oxford, England).

A factor affecting the stability of the DNA double chain includes degree of a basic composition, length and the base pair discordance. That the hybridization condition can be adjusted by those skilled in the art, and it makes these variables applied and different sequence-related DNA forms a hybrid is enabled. The melting temperature of the agreed DNA double chain can be completely estimated by the following formulas roughly. 81.5+16.6 (log [Na+])+0.41 Tm (° C.)=(% G+C)−600/N−0.72 (% formamide) here, N is double stranded length to be formed, and [Na+] is molality of hybridization solution or wash solution sodium ions, and, % G+C is hybrid (guanine+cytosine) basic percentages. About an incompletely agreed hybrid, the melting temperatures decrease by approximately 1 degree Celsius to for each 1% of discordance (mismatch).

Herein, it refers to the condition that the DNA double chain which has base pair discordance of high degree than it can be produced under “a highly stringent condition” with “the moderately stringent condition” can form. The example of a representative “moderately stringent condition” is 0.015M sodium chloride, 0.0015M sodium citrate, 50-65 degrees Celsius or 0.015M sodium chloride, 0.0015M sodium citrate and 20% of formamide, 37-50 degrees Celsius. As an example, the “moderately stringent” condition of 50 degrees Celsius permits approximately 21% of discordance in 0.015M sodium ion.

Herein, it is understood by those skilled in the art that the complete distinction cannot exist between a stringent condition to a stringent condition and “medium degree” to “high level”. For example, in 0.015M sodium ion (there is no formamide), the agreed long melting temperature of DNA is completely approximately 71 degrees Celsius. In irrigation at 65 degrees Celsius (the same ionic strength), this makes approximately 6% of discordance permission. Those skilled in the art can reduce merely temperature, or it can rise with ionic strength to capture spaced-apart related sequence.

About an oligonucleotide probe to approximately 20 nucleotide, the appropriate rough estimate of the melting temperature in 1 MNaCl is provided by Tm=(because of one A-T base 2 degrees Celsius)+(because of one G-C base pair 4 degrees Celsius). Note that sodium ion concentration in 6*citric acid sodium (SSC) is 1M (, also known as referring to Suggs et al., Developmental Biology Using Purified Genes, page 683, Brown and Fox (ed.) (1981)).

The nucleic acid which encodes polypeptide having amino acid sequencing of SEQ ID NO: * or the modification body or a fragment primarily 1% bovine serum albumin (BSA), 500 mM sodium phosphate (NaPO4), 1 mMEDTA, At temperature of 42 degrees Celsius for a hybridization buffer solution including the 7% SDS and essence 2*SSC (600 mM NaCl) 60 mM sodium citrate), A low stringent condition bottom defined by an irrigation buffer solution including the 0.1% SDS of 50 degrees Celsius, more preferably, primarily 1% bovine serum albumin (BSA) at the temperature of 50 degrees Celsius, 500 mM sodium phosphate (NaPO4), 15% formamide, 1 mMEDTA, For a hybridization buffer solution including the 7% SDS and essence 1*SSC (300 mMNaCl) of 50 degrees Celsius 30 mM sodium citrate), A low stringent condition bottom defined by an irrigation buffer solution including the 1% SDS, most preferably, primarily 1% bovine serum albumin (BSA) at the temperature of 50 degrees Celsius, 200 mM sodium phosphate (NaPO4), 15% formamide, 1 mMEDTA, For a hybridization buffer solution including the 7% SDS and essence 0.5*SSC (150 mMNaCl) of 65 degrees Celsius 15 mM sodium citrate), A low stringent condition defined by an irrigation buffer solution including the 0.1% SDS can be hybridized with one or the part of the sequence shown in the array table below.

The amino acid can be mentioned in the present specification by either of the one character sign recommended generally by well-known three characters sign or IUPAC-IUBBiochemicalNomenclatureCommission. The nucleotide can be mentioned likewise by one character cord recognized generally.

Herein, with the genetic “homology”, it refers to degree of the identity to each other of gene arrangement 2 or more. Thus, identity of those sequence or the similarity is high so that a certain binary genetic homology is high. In the case of a direct comparison of the sequence or a nucleic acid, it can be checked whether two kinds of genes have a homology by the hybridization under a stringent condition. When the binary gene arrangement is compared directly, those genes have a homology between the gene arrangement when at least 80%, 90%, 95%, 96%, 97%, 98% or 99% are the more preferably same when DNA sequence is the preferably same at least 70% when at least 50% are the same typically.

In the present specification, the similarity of amino acid sequencing and the sequence, identity and the homologous comparison are calculated using a default parameter using BLAST which is a sequence analytical grade tool. For example, the search of the identity can be performed using BLAST2.2.9 (2004.5.12 publication) of NCBI. Values of the identity in the present specification usually refer to values when it was aligned under conditions of a default using BLAST. However, the highest value is done with a value of the identity when a higher price appears by the change of the parameter. When identity is evaluated in a plurality of regions, the highest value of those is done with a value of the identity.

Herein, amino acid “equivalent” refers to the amino acid that what it has basis of comparison and the action like the protein that it is or the predetermined amino acid in the polypeptide in a certain protein monad or polypeptide monad, or it has is predicted.

Herein, when what it has the action like the predetermined gene in the species that it is basis of comparison in one kind or another with the gene “equivalent”, or it has refers to a predicted gene, and plural genes having such an action exist, it refers to a thing having the same origin for theory of evolution. Thus, the gene corresponding to a certain gene (e.g., NELL1) can be the genetic ortho log. Thus, the gene corresponding to the gene of the Homo sapiens can be found in the other animals (a mouse, an albino rat, a swine, a rabbit, Cavia, cattle, Ovis). The gene giving such a response can be identified using a technique well known in the art. Thus, for example, it can be found by the corresponding gene in a certain animal uses a coping genetic criterion and the genetic sequence that it is as query sequence, and retrieving the sequence database of the animal (e.g., a mouse, an albino rat, a swine, a rabbit, Cavia, cattle, Ovis).

Herein, with “a section” or “the fragment”, it refers to polypeptide having sequence length to 1-n-1 or polynucleotide to polypeptide of the full length or polynucleotide (length n). The length of the fragment can be changed depending on the object appropriately, and, for example, in the case of polypeptide, 3, four or five, six or seven, eight or nine, 10, 15, 20, 25, 30, 40, 50 and a further amino acid are included, and length (e.g., 11) represented with the integer that is not enumerated specifically of here can be appropriate as the lower limit for the lower limit of the length in turn. Also, in the case of polynucleotide, 5, six or seven, eight or nine, 10, 15, 20, 25, 30, 40, 50, 75, 100 and further nucleotide are included, and length (e.g., 11) represented with the integer that is not enumerated specifically of here can be appropriate as the lower limit in turn. Herein, the length of polypeptide and the polynucleotide can be represented by the number of an amino acid or the nucleic acid like statement above, respectively, but as far as the number is not absolute, and it has the same function, as for the number as the upper limit or the lower limit, what the thing of several fluctuations (or, for example, 10% of fluctuations) of the number includes is aimed at. “About”, in the present specification, [symbol] is attached before the number, and it may be expressed to express such an intention. However, in the present specification, it should be understood that “about”, it does not have an influence on interpretation of the [symbol] numerical value. It can be determined whether at least one function is maintained among functions of full length protein becoming the criterion of the fragment as for the length of a fragment useful herein.

Herein, “the homology” shows that those sequence shares an ancestor evolutionarily in a comparison of sequence 2 or more. A judgment based on a similarity by a direct comparison of the sequence is performed, or it can be examined whether two kinds of genes have a homology by the hybridization under a stringent condition. When a judgment based on a similarity is performed by a direct comparison of the sequence, character string juxtaposed by character string (a base, an amino acid residue) that is the same as the simplest interpretation in the alignment of the similarity measurement process (or it is equivalent) shows that these regions did not change as ancestor sequence, and discussion that a mutation is one sequence, and the character string that is nonidentical (or it is not equivalent) was provided is possible. A gap (in Dell) in the alignments is one side of the sequence, and it is thought that intercalation or deletion was generated. In other words it is understood that it strongly suggests a homology in those sequence that identity of those sequence or the similarity is high. The homology is referred to in the design of the expression inhibitor.

Herein, it “is complementary” or the whole complementary region shows sequence of the polynucleotide which can just form another particular polynucleotide and Watson & Crick base pair in the term “complement” in the present specification. For purposes of this invention, it is considered when each base of the first polynucleotide matches with the complementary base when this first polynucleotide is the second polynucleotide and complementary. Generally the complementary base is A and T-head (or A and or C and G. In the present specification, a word “complementary” is used as a synonym of “complementary polynucleotide”, “a complementary nucleic acid” and “the complementary nucleotide sequence”. These terms are applied to a pair of the polynucleotide only based on the sequence, and binary polynucleotide is not applied to a particular set in integrated state virtually.

Herein, with “the modified body”, a part refers to a changed thing to materials such as original polypeptide or the polynucleotide. Such a modified body includes a substituted modified body, an addition modified body, a deficiency modification body, a shortening (truncated) modification body, an allelic variant. It belongs to a gene locus same as the allele (allele), and it refers to a hereditary modification body distinguished each other. Thus, with “the allelic variant”, it refers to a modification body having an allelic relation to a certain gene. With “species homolog or the homolog” (homolog), it refers to a thing having a homology (homologies preferably 60% or more more preferably the homology that is 95% or more more than 90% more than 85% more than 80%) in the gene which there is and an amino acid level or a nucleotide level in one kind or another. The method to acquire such a species homologue is clear from description of the present specification. The orthologous gene (orthologousgene) is said, and, with “the ortho log” (ortholog), it refers to a gene coming from speciation from the common ancestor with two genes. For example, Homo sapiens and the murine a hemoglobin gene are ortho log when the hemoglobin gene family having a multigene conformation is taken for an example, but a a hemoglobin gene and the β hemoglobin gene of the Homo sapiens are para-log (the gene which occurred in gene duplication). Because the ortho log is useful for an estimate of the molecular dendrogram, the ortho log can be also useful herein, too.

Herein, “the conservative (modified) modified body” is applied to both amino acid sequencing and nucleotide sequences. When it refers to a nucleic acid encoding of the equivalence or the primarily same amino acid sequencing with the modification body modified conservatively with reference to a particular nucleotide sequence, and a nucleic acid does not encode amino acid sequencing, it refers to sequence same primarily. A modified method of such a sequence includes a site-directed base substitution method (specific area-oriented mutation method, Mark Zoller and Michael Smith, Methods in Enzymology, 100,468-500 (1983)) using the amputation with restriction enzymes, DNA polymerase, a Klenow fragment, the processing such as connections by the processing by the DNA ligase, the synthetic oligonucleotide, but it can be modified by a method used in an usually, in addition, molecular biological field. For degeneracy of the genetic code, a large number of nucleic acids same functionally encode any predetermined protein. For example, all codon GCA, GCC, GCG and GCU encode amino acid alanine. Thus, at all positions where alanine is identified by a codon, the codon can be changed to the arbitrary thing of a described corresponding codon without changing encoded polypeptide. A change of such a nucleic acid “is the silent modification” that is a species of variable one modified conservatively (a transmutation). All nucleotide sequences of the present specification to encode polypeptide also describe all possible silent change of the nucleic acid. In the art, it is understood that it can be modified because each codon (except AUG which is usually the only codon for methionine and TGG which are an exclusive codon for conventional tryptophan) in a nucleic acid produces the same monad functionally. Thus, each silent change of a nucleic acid encoding polypeptide is included in each described sequence tacitly. Preferably such a modification can be done to evade a substitution of the Cys which is the amino acid which has a great influence on a higher-order structure of the polypeptide.

For example, a certain amino acid can be substituted for other amino acids in the protein structure such as the binding site of the ligand monad without a clear reduction of the coaction binding capacity or an ablation. It is proteinaceous ability for coaction and character that prescribe a certain proteinaceous biological function. Thus, the substitution of the particular amino acid can be performed in the level of in amino acid sequencing or the DNA sequence, and protein maintaining still original character after a substitution can be produced. Thus, without the clear loss of the biological utility, various kinds of modifications can be performed in corresponding DNA encoding peptide disclosed herein or this peptide.

When the modification such as the above is designed, the hydrophobic index of the amino acid can be considered. The importance of the hydrophobic amino acid index when an interactive biological function in the protein is provided is recognized generally in the art (Kyte. J and Doolittle, R. F. J. Mol. Biol. 157 (1): 105-132, 1982). The hydrophobic property of the amino acid contributes to a formed secondary structure of protein, and subsequently the coaction with the protein and other monads (e.g., an enzyme, a matrix, an acceptor, DNA, an antibody, antigen) is prescribed. Each amino acid is assigned a hydrophobic index based on the character of those hydrophobicity and electric charge to. They: Isoleucine (+4.5), A 2-amino-3-methylbutyric acid (+4.2), Leucine (+3.8), Phenylalanine (+2.8), Cys/cystine (+2.5), Methionine (+1.9), Alanine (+1.8), Glycine (−0.4), Threonine Serine (−0.8), Tryptophan (−0.9), Tyrosine (−1.3), Proline (−1.6), Histidine (−3.2), Glutamic (−3.5), Glutamine (−3.5), An asparaginic acid (−3.5), Asparagine (−3.5), Lysin (−3.9), And it is arginine (−4.5)).

What can cause protein (the protein which, e.g., is equivalent in ligand binding capacity) which and a certain amino acid is substituted by other amino acids having a similar hydrophobic index and has a still similar biological function is well known in the art. In such an amino acid substitution, it is even more desirable that more preferred, and it is less than +−0.5 that it is preferably less than +−1 that a hydrophobic index is less than +−2. It is understood that the substitution of such an amino acid based on the hydrophobicity is effective in the art. The following hydrophilic property indexes are assigned to an amino acid residue so that it is described in U.S. Pat. No. 4,554,101: Arginine (+3.0), Lysin (+3.0), An asparaginic acid (+3.0+−1), Glutamate (+3.0+−1), Serine (+0.3), Asparagine (+0.2), Glutamine (+0.2), Glycine (0), Threonine (−0.4), Proline (−0.5 +−1), Alanyl (−0.5), Histidine (−0.5), Cys (−1.0), Methionine (−1.3), A 2-amino-3-methylbutyric acid (−1.5), Leucine (−1.8), Isoleucine (−1.8), Tyrosine (−2.3), Phenylalanine (−2.5), And tryptophan (−3.4). It is understood that it can be substituted for another which an amino acid has a similar hydrophilic property index and can still provide a biological equivalent. In such an amino acid substitution, it is even more desirable that more preferred, and it is less than +−0.5 that it is preferably less than +−1 that a hydrophilic index is less than +−2.

In the present invention, with “the conservative substitution”, it refers to the hydrophilic property index with an original amino acid and a substituted amino acid or/and a substitution resembled in a hydrophobicity index as discussed above in amino acid substitution. The example of the conservative substitution is well known to those skilled in the art, for example, it is the substitution in each group of the next: Arginine and lysin, Glutamate and an asparaginic acid, Serine and threonine, Glutamine and asparagine, And a 2-amino-3-methylbutyric acid, leucine and isoleucine are included, but is not limited to these.

Herein, other than the substitution of the amino acid, the addition of the amino acid, a deficiency or the modification can be also performed to make equivalent polypeptide functionally. With the substitution of the amino acid, it refers to substituting original peptide for more than one, e.g., the amino acid of 1-10 preferably 1-5 more preferably 1-3. With the addition of the amino acid, it refers to adding more than one, e.g., the amino acid of 1-10 preferably 1-5 more preferably 1-3 to original peptide chain. With the deficiency of the amino acid, it refers to deleting more than one, e.g., the amino acid of 1-10 preferably 1-5 more preferably 1-3 from original peptide. The amino acid modification includes amidation, carboxylation, a sulfate, halogenation, alkylation, phosphorylation, hydroxylation, acylation (e.g., it is acetylated), but is not limited to these. A substitution or an added amino acid may be a natural amino acid, and even a non-natural amino acid or an amino acid analog is preferable. A natural amino acid is preferable.

Such a nucleic acid can be obtained by the well-known PCR method, and it can be synthesized chemically. For example, for these methods, site-directed displacement elicitation, hybridization may be put together.

Herein, with “a substitution, addition and/or the deletion” of polypeptide or the polynucleotide, it refers to an amino acid or the alternatives or nucleotide or the alternatives being moved to original polypeptide or polynucleotide, respectively, adding or being removed. The technique of such a substitution, addition and/or the deletion is well known in the art, and, for such a technical example, site-directed mutagenesis techniques are included. As far as, as for these changes in nucleic acid molecule becoming the criterion or the polypeptide, a function (e.g., ossifications) to be intended is maintained, it can be produced in 5′ end of this nucleic acid molecule or 3′ end, or it can be produced in an amino terminus area of the amino acid sequencing showing this polypeptide or a carboxyl terminal area, or it can be produced anywhere between a thing of extremity areas thereof, and it lies scattered between reference sequence residues individually. As far as if a substitution, addition or a deficiency is one or more, as for such number, the substitution, addition or function (e.g., recognition ability of AGE) to be intended in a modification body having a deficiency is well maintained with any number, it can be increased. For example, it can be 1 or several and such a number can be preferably lower than of the overall length less than 20%, less than 15%, less than 10%, less than 5% or 150, lower than 100, lower than 50, lower than 25.

Herein, with “the cell”, is made the usage in the art, is used like the meaning of the wide sense most, and it is in form comprising an organism and/or a functional fundamental unit.

Herein, with “the tissue”, is made the usage in the art, is used like the meaning of the wide sense most, and it is said by cellular collection to have the substantially same form, a function. In the animals, it is distinguished to epithelial tissue, connective tissue, a muscle tissue, nervous tissue.

Herein, with “the organ”, is made the usage in the art, is used like the meaning of the wide sense most, and, in a multicellular organism, tissue gathers, and it has constant form, and it is said in a part running special functions such as respiration, excretion, the digestion. Herein, it can trade with “an organ” and “the organ”, and it can be used.

Herein, it refers to a cell, tissue or an organ being formed in the locus which does not originally exist to “form ectopically”. For example, as an ectopic plastic example, formation of the blood forming tissue in the adipose tissue, formation of the epithelium system tissue in the adipose tissue, formation of the blood forming tissue in the muscular tissue, epithelium system tissue in the muscular tissue can be raised, but is not limited to these.

Herein, it refers to an existing thing to “maintain” a cell, tissue or an organ with a cell, tissue and an organ keeping a function of the form and at least one copy.

Herein, with “the environment with adipose tissue and a blood vessel maintaining it”, adipose tissue and the adeps form and a blood vessel to maintain a function refer to existing environment. For example, environment around the furcation of the lower abdominal wall status pulse in the adipose tissue of the abdominal regions subcutis is included.

Herein, with “the environment with muscular tissue and a blood vessel maintaining it”, muscular tissue and the sarcous form and a blood vessel to maintain a function refer to existing environment. For example, arteriovenous environment in the muscular tissue of the femoral region is included.

Herein, with “the cell that orientation of constant differentiation was accomplished”, it refers to the cell which there is in the middle of a determined differentiation stage to differentiate to a particular cell. For example, in the present specification, cellular precursor cells comprising the somatic stem cell which there is to tissue (e.g., blood forming tissue, epithelial tissue, muscular tissue, nervous tissue) and tissue are included, but is not limited to these.

Herein, with “the stem cell”, is made the usage in the art, is used like the meaning of the wide sense most, and pass by cell division, and it is said with a cell maintaining the same differentiation potency.

Herein, with “the somatic stem cell”, is made the usage in the art, because is used like the meaning of the wide sense most, and all tissue/organs always perform remodelling (an absorption/rebuilding), it is said with the cause to comprise found tissue and the cell that it is. Herein, with “a somatic stem cell” and “a tissue stem cell” and “the imago stem cell”, it can be changed, and it can be used. Somatic stem cells include, but are not limited to, somatic stem cells which, for example, there is to blood forming tissue, epithelial tissue, connective tissue, muscular tissue, nervous tissue.

As for the somatic stem cell, orientation of the differentiation was done to some extent by an all-around stem cell seen in embryonic stem cell and iSP cell. Therefore a name is touched every hematopoietic stem-cell, mesenchyme system stem cell, main tissue including the truncal cell. However, that a somatic stem cell causes dedifferentiation (antidromic differentiation) is expected. For example, with a report detected tissue of the donor after grafting of “the hematopoietic stem-cell” by the bone marrow transplantation performed by a clinic by the liver of the recipient (Alison M R, Hepatocytes from non-hepatic adult stem cells., Nature. 2000 Jul. 20, 406 (6793): 257). Thus, a somatic stem cell or a universal stem cell differentiates, and, as well as a somatic stem cell, it is in “hematopoietic stem-cell”, and blood corpuscles can further differentiate. The present invention can use these stem cells appropriately.

For example, the somatic stem cell which there is to blood forming tissue includes hematopoietic stem-cell, a myeloid stem cell. It is the stem cell that orientation of the differentiation was done to epithelial tissue, and the somatic stem cell which there is to epithelial tissue is metrocyte of an epithelium system cell having self-repair ability and multipotency. For example, the somatic stem cell which there is to epithelial tissue includes glandular original cells, a cell included in the hair-root. For example, as for the somatic stem cell which there is to this epithelial tissue, a gland cell or a cell of the hair-root can differentiate.

Herein, with “the hematopoietic stem-cell”, it refers to the metrocyte of a blood cell having self-repair ability and multipotency, and, for example, the thing which flows out into the peripheral blood from bone marrow with the cytokine such as granular colony stimulating factors (G-CSF), every blood cell include the things which differentiate (having totipotency).

For example, the hematopoietic stem-cell is used by the treatment (e.g., hematopoietic stem-cell is transplanted) to the leukemic patient. Originally the hematopoietic stem-cell grafting began with a bone marrow transplantation. Because it was with blood cell blood cell of the patient all derived from donor when bone marrow was transplanted from the brothers who agreed of the HLA after chemotherapy in large quantities in the latter half of 1960, a bone marrow transplantation was devised. Then it develops that hematopoietic stem-cell comes out in peripheral blood when G-CSF is administered, and the peripheral blood stem cell grafting using this is performed from 1990's. Even more particularly, that umbilical blood included much hematopoietic stem-cell for the same period was understood, and umbilical blood grafting came to be performed mainly on child. The present invention can use the technique about these stem cells appropriately.

Herein, with “the blood forming tissue”, is made the usage in the art, is used like the meaning of the wide sense most, and various kinds of blood corpuscles and a materiality ingredient are formed, and it is said with tissue to develop.

Herein, with the cell of “the hematopoietic system”, tissue or the organ, is done the usage in the art, is used like the meaning of the wide sense, and it is the generic designation of various kinds of cells, tissue comprising blood forming tissue or the organ most.

Herein, with “the epithelial tissue”, is made the usage in the art, is used like the meaning of the wide sense most, and it is said in a cell layer covering a zoic body surface and the intracorporeal organ inner surface.

Herein, with the cell of “the epithelial system”, tissue or the organ, conventional in the art, is done, is used like the meaning of the wide sense most, and it is the generic designation of a cell, tissue forming an epithelium or the organ.

Herein, with “the glandular original cells”, orientation of the differentiation refers to a done stem cell to epithelial tissue existing in a cellular aggregate (a gland) running a secretion.

Herein, with “the cell included in the hair-root”, it refers to the cell included in a hairy part buried in folliculi pili. There is a cell directed that it differentiates by hair-root to a cell included in this hair-root.

Herein, with “the exocrine gland”, is made the usage in the art, is used like the meaning of the wide sense most, and it is said with a gland released secreted material in the body surface by a pipe. For example, an exocrine gland includes a perspiratory gland, sebaceous glands, an intestinal gland, a gastric gland, a salivary gland. All glandular systems are comprised of the epithelium of the cube from a column having secretional capacity, and the secreted material is different by each tissue (an organ). Thus, a secretory gland is extracted from a desired organ, and a desired exocrine gland can be obtained by locating to the organism incubator of the present invention.

Herein, with “the connective tissue”, is made the usage in the art, is used like the meaning of the wide sense most, and a zoic body is supported, and it is said with tissue formed from a framework and the tissue that it is, the fiber with various cells and a matrix. The connective tissue comes from mesenchyme occurring from mesoderm. For example, connective tissue includes lymphatic tissue, cartilage, a bone.

Herein, with “the muscular tissue”, is made the usage in the art, is used like the meaning of the wide sense most, and it is the tissue which can shrink to a stimulation. The muscular tissue is distinguished to skeletal muscle, cardiac muscle, smooth muscle tissue.

Herein, with “the nervous tissue”, is made the usage in the art, is used like the meaning of the wide sense most, and it is said with tissue comprising a nervous system. The nervous tissue is constructed by a neuron conveying an excitation (a signal) and neuroglia in support of it

Herein, “the anchorage” means some kind of entities becoming basic for an object, and, for example, what is relied upon running a spatial clue and the thing that it is or some kind of action is included. Herein, or a biological material or a material supplied by a nature, a naturally existing material are synthetic, and the anchorage is made from a supplied material having biocompatibility. Herein, “the anchorage” can be distinguished from “a functional anchorage” in “a controlled-release anchorage”.

Herein, with “the functional anchorage”, it refers to a thing becoming the clues such as a cell, the tissue. For example, in one embodiment, the functional anchorage as used herein may be in vivo tissue (e.g., adipose tissue). It can be referred to as the structural anchorage.

Herein, NELL-1 is held, and “the controlled-release anchorage” means materials to make a controlled release done. For example, in one embodiment, as for the controlled-release anchorage as used herein, extracellular matrices (e.g., collagen sponge, an atelocollagen gel) are included, but is not limited to these. Because NELL-1 is maintained, and the reason is because it should be able to be provided to a cell over while it is to a cell, tissue, the organ which a cell further differentiates, and were derived.

Herein, with “the extracellular matrix” (electro chemical machining), is made the usage in the art, is used like the meaning of the wide sense most, and it is said in a supermolecule structure filling an extracellular space. For example, an extracellular matrix includes collagen, atelocollagen, proteoglycan (e.g., chondroitin sulfated proteoglycan, heparan sulfate proteoglycan, keratan sulfated proteoglycan, dermatan sulfate proteoglycan), hyaluronate, fibronectin, laminin, tenascin, entactin, elastin, fibrillin, but is not limited to these. Herein, it can trade with “an extracellular matrix” and “the extracellular matrix”, and it can be used.

Herein, with “the collagen”, is made the usage in the art, is used like the meaning of the wide sense most, and it is the active ingredient of the zoic extracellular matrix. According to the present invention, for example, the collagen sponge which is available from white Arabian bird sales agency Shiromizu trade Co., Ltd. Co., Ltd. (Osaka-shi), collagen gel, an atelocollagen gel are available, and, for collagen, the collagen from other sources of supply is also available in the present invention.

Herein, with “the medical device”, it refers to an appliance to use in a medical care (e.g., it cures operations) object for the purpose of object to improve a physical function or intervention. The medical device as used herein holds NELL-1, and a cell, tissue, an organ should be able to trigger the NELL-1. The configuration which the medical device as used herein is manufactured in biocompatible materials and can transplant in an organism, a size thing are desirable, and it is desirable not to have immunorejection.

Herein, “the incubator in the organism” is the thing including a mold (e.g., a silicon mold), NELL-1 and the caulescent tissue piece. Herein, with “the caulescent tissue piece”, a blood vessel maintaining a tissue piece and the tissue piece is included, and a blood flow refers to the structure which is in a state which is not obstructed. For example, a caulescent tissue piece includes blood vessels maintaining adipose tissue and blood vessel, muscular tissue maintaining it and it, but if such a character seems to be met, the tissue except the above can be used. Is not restricted by theory, but if the character is met, the reason is because the cell that oxygen is provided by tissue, and a necrosis being inhibited by a blood flow, orientation of also constant differentiation were done is provided.

It can make a functional anchorage (e.g., collagen sponge, an atelocollagen gel) releasing NELL-1 in sustained release included to the incubator in this organism. Because the incubator in the organism is because more differentiated induced cells can be produced only if NELL-1 can be provided to the cell for the period when it is necessary a cell (a, e.g., somatic stem cell) further differentiates, and to guide. In other words the provision tool is preferable with any kind of thing if NELL-1 can be provided to a cell continuously. In one embodiment, NELL-1 can be provided by a part of the caulescent tissue piece (e.g., adipose tissue) by doing addition (e.g., it is injected) directly.

The configuration which it is invivo, and the incubator in the organism as used herein is manufactured in biocompatible materials and can transplant, a size thing are desirable, and it is desirable not to have immunorejection.

Herein, “the mold” means the container which can be ported in an organism. In the present specification, mold manufactured with silicon is called “a silicon mold”. The mold can make a thing to the form of the organ of the grafting. For example, the thing of the rat bone tip configuration and a pine nut-shaped thing using the clay are manufactured, and it can be used. Because the mold holds NELL-1, and a cell, tissue, an organ should seem to be able to trigger the NELL-1. The configuration which the mold as used herein is manufactured in biocompatible materials and can transplant in an organism, a size thing are desirable, and it is desirable not to have immunorejection.

### A Preferred Embodiment

A preferred embodiment of the present invention is described below. The embodiment that is provided below is provided for better understanding of the present invention, and what should not be limited to the following description is understood by the range of the present invention. Thus, as for those skilled in the art, taking into consideration does the description of the present specification, and what can modify ad libitum within the present invention is apparent.

(The Constituent of Differentiation Induction Applications)

In one situation, the present invention makes it differentiates, and the cell that orientation of constant differentiation was done guided, and a constituent to do to a more differentiated induced cell, tissue or an organ in way of the said differentiation is provided. This constituent can include NELL-1 or the matter which is changed into to function as NELL-1 at a point of said differentiation. Even if it is any kind of organ, according to the constituent of the present invention, it can be produced. Ogawa K, Living donor liver transplantation with reduced monosegments for neonates and small infants. Transplantation. 2007 May 27, 83 (10): To 1337-40, possible presence is described in a donor liver or a recipient liver a somatic stem cell given a deep significance to in being differentiated by a liver after pediatric liver transplantation performed by a clinic. Thus, allopatric histodifferentiation with the present invention or the retention of the liver tissue piece, the growth are not restricted by theory, but an existing cell repeats remodelling to NELL-1 and touched tissue (including the explant), and the reason is because it is thought that possibility repeating growth only with an auto cell or an undifferentiated cell included in a tissue piece multiplies.

Herein, the cell that orientation of constant differentiation was accomplished may be any kind of cell. Is not restricted by theory, but, in NELL-1 as used herein, more differentiated with the cell to a cell determined a polarity of the differentiation, if because it acts to make guided, as for the cell to intend for, a polarity of the differentiation is a constant cell, as for what kind of cell the reason is because it may be.

For example, in one embodiment, the material that is changed to function as NELL-1 at a point of NELL-1 or said differentiation can be the material which can produce NELL-1 using the gene engineering techniques such as plasmid, the viral vector, but is not limited to these. The reason is because it can make it further differentiates, and the cell guided only if NELL-1 can be triggered to the cell that orientation of constant differentiation was accomplished.

In one embodiment, way of the differentiation can be a constituent to form a more differentiated induced cell, tissue or an organ ectopically in the environment with the blood vessel that the constituent of the present invention maintains adipose tissue and it.

In a preferred embodiment, for example, environment with adipose tissue and a blood vessel maintaining it is environment such as the environment around the furcation of the lower abdominal wall status pulse in the adipose tissue of the abdominal regions subcutis, but is not limited to these.

In the present invention, the reason why environment with the adipose tissue is preferable is not restricted by theory, but the reason is because it differentiates, and a stem cell (Zuk P A, Multilineage cells from human adipose tissue: implications for cell-based therapies. Tissue Eng. 2001 April, 7(2)211-28.) derived from adipose tissue may be derived by NELL-1.

The somatic stem cell seen in adipose tissue may differentiate to a mesodermal tissue (steatogenesis, a chondrogenic myogenic and bone morphogenetic cell). It is a document reinforcing allopatric histodifferentiation with the present invention and the maintenance of the liver tissue piece, productive possibility. However, because the range where fat-cell can differentiate is extremely limited with this thesis, it foresees by epithelial tissue development with the present invention, it is not particularly. A point at issue slips off, but because, for example, blood vessel muscular tissue (performed a lot by an oral surgery clinic) belonging to can be put in a silicon mold, and the somatic stem cell that the orientation which, in that case, differentiates to muscular tissue was done exists, and, not the drawn game that environment with the adipose tissue is desirable, these are mesoderm systems (muscular tissue), possibility to differentiate is hidden in a mesoderm pro-cell.

However, the possibility that a somatic stem cell of the distinction is sent into through nutritional done tissue (in fat or muscular tissue) body fluid circulation by a regular blood vessel is extremely high, and that allopatric tissue occurs in a silicon mold will be supported so that the following Alison M R article has. Because the present invention mainly comes up with the idea of storing a blood vessel and composition (e.g., muscular fat) in a capsule for perfection and efficiency, the information that it is necessary to consider “new result” produced for an experimental result (histology) that the thesis of the series obtained by the present invention rather than the reason that is essential to the present invention is provided.

The reason why the environment with the blood vessel is preferable is not restricted by theory, but because there is report (Alison M R, Hepatocytes from non-hepatic adult stem cells., Nature. 2000 Jul. 20, 406 (6793): 257) that the patient hepatic cell after the bone marrow transplantation included a cell derived from donor, the reason is because it differentiates, and an existing somatic stem cell may be guided to another place through body fluid circulation by NELL-1.

In another embodiment, the cell that orientation of the constant differentiation as used herein was accomplished can be the somatic stem cell which there is to tissue such as a somatic stem cell, e.g., blood forming tissue, epithelial tissue, connective tissue, muscular tissue, the nervous tissue. Preferably it can be blood forming tissue, epithelial tissue, connective tissue. Based on a well⁻known bibliography, a well-known bibliography, those skilled in the art select appropriately, and the somatic stem cell as used herein can be prepared.

In a preferred embodiment, for example, the arriving tissue (an organ) can manufacture life in the adipose tissue in the incubator in the organism of the present invention as follows:

1. A method to disseminate a cell coming from the organ (e.g., liver pieces) of an auto or others. In these organs, both mature cell and somatic stem cells is included.

2. A method to provide hematopoietic stem-cell (distributed in peripheral blood) through the somatic stem cell which there is to adipose tissue and a connected blood vessel. From these, for example, a hematopoietic system tissue, a glandular system can be formed.

3. A method to disseminate the culture cells which orientation of the differentiation was made. (such a culture cell is available from the tissue such as the cell bank by purchasing or preparing.).

4. Form the organ from a stem cell by mixing the material which a stem cell is disseminated, and it differentiates therein, and is directed.

according to the present invention, by the experiment that compared the thing which both FGF and NELL-1 were added to, NELL-1 cooperated with other growth factors or it competed, and an available thing was proved that a fibroblast growth factor (FGF) was added in an invivo incubator. Thus, because it was already directed the differentiation or A) directs NELL-1 by an application regardless of the illustration that enumerated in sections 1 to 4 above, differentiation guides the cell to the cell that orientation of constant differentiation as used herein was done more, and it can be implemented differentiated promoted thing and to promote high cellular take of the B) differentiation degree.

in another embodiment, the stem cell which there is to the blood forming tissue used in a constituent of the present invention can be metrocyte of a blood cell having self-repair ability and multipotency. For example, the stem cell which there is to this blood forming tissue can be guided blood cell (e.g., an erythrocyte, leucocyte, a macrophage) differentiation to by the cytokine such as granular co-low knee stimulating factors (G-CSF).

in another embodiment, the somatic stem cell which there is to the epithelial tissue as used herein can be glandular original cells, a hair-root cell, but is not limited to these.

in another embodiment, the differentiation induced cell with the present invention, tissue or an organ can be a hematopoietic system, the cells such as epithelium systems, tissue or an organ, but is not limited to these.

in another embodiment, a cell, the tissue which it differentiates, and were derived by the hematopoietic system with the present invention or the organ can be blood corpuscles such as from neutrophils, eosinocyte, basophils, leucocyte such as the lymphocyte, an erythrocyte, blood platelet, a macrophage, those combinations, but is not limited to these. Is not restricted by theory, but even if it makes NELL-1 dedifferentiates a mature cell, and a hematopoietic system cell redifferentiate, it is conceivable, but a constituent of the present invention acts in hematopoietic stem-cell, and, according to orientation of the differentiation, differentiation guides hematopoietic stem-cell more, and possibility done to a hematopoietic system cell is considered.

the constituent of the present invention can form the cell of the hematopoietic system, tissue and an organ in allopatry. The cell of the differentiation induced hematopoietic system, tissue and the organ have prominent effect outside expectation to be able to run the function that they originally have by the present invention.

in another embodiment, a cell, the tissue which it differentiates, and were derived by the epithelial system with the present invention or an organ can be an exocrine gland and the pipe, but is not limited to these. For example, this exocrine gland includes a perspiratory gland, sebaceous glands, an intestinal gland, a gastric gland, a salivary gland, but is not limited to these. Is not restricted by theory, but even if it makes NELL-1 dedifferentiates a mature cell, and an epithelial system cell redifferentiate, it is conceivable, but it acts, and differentiation guides an epithelium stem cell to the somatic stem cell that a constituent of the present invention exists in an epithelium according to orientation of the differentiation more, and possibility done in a glandular system is considered. In a preferred embodiment, the cell that orientation of the constant differentiation was accomplished is glandular original cells, and the differentiated induced cell, tissue or an organ can be an exocrine gland, the pipe.

the constituent of the present invention can form a glandular system in allopatry. The differentiation induced glandular system has prominent effect outside expectation to be able to run the function that they originally have by the present invention.

in another embodiment, the epithelial system composition with the present invention can be a glandular system accompanying hair, bulbus pili, hair-root, hair-root, but is not limited to these. Is not restricted by theory, but even if it makes NELL-1 dedifferentiates a mature cell, and an epithelial system cell redifferentiate, it is conceivable, but it acts, and differentiation guides a hair-root cell to the somatic stem cell (a hair-root cell) that a constituent of the present invention exists in hair-root according to orientation of the differentiation more, and possibility done in an appendant glandular system in hair, bulbus pili, hair-root, hair-root is considered. In a preferred embodiment, the cell that orientation of the constant differentiation was accomplished is a cell included in the hair-root, and the differentiated induced cell, tissue or the organ can be a glandular system accompanying hair, bulbus pili, hair-root, hair-root.

the constituent of the present invention can form a glandular system accompanying hair, bulbus pili, hair-root, hair-root in allopatry. The tissue of differentiation these induced has prominent effect outside expectation to be able to run the function that they originally have by the present invention.

in another embodiment, quantity of NELL-1 included in the constituent of the present invention can be about 0.01 μg/ml-approximately 1,000 μg/mL, about 0.1 μg/ml-approximately 100 μg/mL preferably about 5 μg/ml-approximately 50 μg/mL, about 5 μg/ml-approximately 10 μg/mL more preferably approximately 5 μg/mL more than approximately 0.01 μg/mL. For example, the quantitative lower limit of this NELL-1 can be approximately 0.01 μg/mL, approximately 0.1 μg/mL, approximately 0.2 μg/mL, approximately 0.3 μg/mL, approximately 0.4 μg/mL, approximately 0.5 μg/mL, approximately 0.6 μg/mL, approximately 0.7 μg/mL, approximately 0.8 μg/mL, approximately 0.9 μg/mL, approximately 1.0 μg/mL, approximately 1.5 μg/mL, approximately 2.0 μg/mL, approximately 2.5 μg/mL, approximately 3.0 μg/mL, approximately 3.5 μg/mL, approximately 4.0 μg/mL, approximately 4.5 μg/mL, approximately 5.0 μg/mL, approximately 5.5 μg/mL, approximately 6.0 μg/mL, approximately 6.5 μg/mL, approximately 7.5 μg/mL, approximately 8.0 μg/mL, approximately 8.5 μg/mL, approximately 9.0 μg/mL, approximately 9.5 μg/mL, arbitrary numerical value of about 0.01 μg/ml-approximately 1,000 μg/mL including approximately 10.0 μg/mL. For example, the quantitative upper limit of this NELL-1 can be approximately 10.0 μg/mL, approximately 9.5 μg/mL, approximately 9.0 μg/mL, approximately 8.5 μg/mL, approximately 8.0 μg/mL, approximately 7.5 μg/mL, approximately 6.5 μg/mL, approximately 6.0 μg/mL, approximately 5.5 μg/mL, approximately 5.0 μg/mL, approximately 4.5 μg/mL, approximately 4.0 μg/mL, approximately 3.5 μg/mL, approximately 3.0 μg/mL, approximately 2.5 μg/mL, approximately 2.0 μg/mL, approximately 1.5 μg/mL, approximately 1.0 μg/mL, approximately 0.9 μg/mL, approximately 0.8 μg/mL, approximately 0.7 μg/mL, approximately 0.6 μg/mL, approximately 0.5 μg/mL, approximately 0.4 μg/mL, approximately 0.3 μg/mL, approximately 0.2 μg/mL, approximately 0.1 μg/mL, arbitrary numerical value of about 0.01 μg/ml-approximately 1,000 μg/mL including approximately 0.01 μg/mL. Even if this numerical value is put in the exocrine gland (e.g., a perspiratory gland, sebaceous glands, an intestinal gland, a gastric gland, a salivary gland) of the cellular (e.g., neutrophils, eosinocyte, basophils, leucocyte such as the lymphocyte, an erythrocyte, blood platelet, a macrophage) epithelial system of the hematopoietic system, hair, bulbus pili, hair-root, the hair-root in the case of appendant glandular systems, it is similar. Those skilled in the art can put the quantity of NELL-1 to incorporate into a constituent of the present invention together for the applications, and it can be decided appropriately, and the density can be determined definitely.

in another embodiment, the differentiation induced cell, tissue or the organ has a corresponding function to exist naturally. This function includes, for example, the following things, and those functions can be confirmed as follows.

A Hematopoietic System Tissue:

It is confirmation of the histology by the tissue staining (hematoxylin eosin staining), the secretory confirmation that are enumerated below the thing in confirmation of the expression of the cellular marker of the hematopoietic system, an exocrine gland and the pipe by immunochemistry staining and furo-site met Lee

A Perspiratory Gland:

Confirmation of secreted material such as sodium and the apocrine secretion (apocrine secretion)

Sebaceous Glands:

Secretory Confirmation such as the Lipid

An intestinal gland: Secretory confirmation such as ion peptidase P., the maltase

A gastric Gland:

Confirmation such as the pepsinogen such as digestive enzymes or a proton or the chloride

A Salivary Gland:

A glandular system appendant in secretory confirmation hair of amylase, ptyalin or the peroxidase, bulbus pili, hair-root or hair-root: The observation due to the unaided eye, a constituent of the confirmation present invention of the histology by the tissue staining (hematoxylin eosin staining), the materials can include as necessary appropriate medication materials or a pharmaceutically acceptable carrier. Appropriate medication materials or a pharmaceutically acceptable carrier includes anti-oxidant, preservative, a coloring agent, fluorescent dye, a flavoring agent, diluent, an emulsifier, a suspending agent, a vehicle, a filler, an extending agent, a buffer, a delivery vehicle and/or adjuvant of the pharmacy, but is not limited to them. It is administered other active principle to the constituents of the present invention in the form of a containing constituent or materials with at least one physiologically acceptable carrier, diluting agent or diluent depending on NELL-1 and a need typically. For example, an appropriate vehicle can be micell, an injection solution, a solution of the physiology or artificial cerebrospinal fluid, and a material of the commonly used others can be supplemented to a constituent for parenteral delivery in these.

it is inert, and, for example, as for the carrier, diluting agent which can be received as used herein or the stabilizer, phosphate, citrate or other organic acid, ascorbate, alpha-tocopherol, low molecular weight polypeptide, protein (e.g., serum albumin, gelatine or Ig), a hydrophilic polymer (e.g., polyvinylpyrrolidone), an amino acid (e.g., glycine, glutamine, asparagine, arginine or lysin), monosaccharide, disaccharide and other carbohydrates (D-glucopyranose, mannose or dextrin), a chelating agent (e.g., EDTA), glycitol (e.g., mannitol or sorbitol), salt formation counterion (e.g., sodic) and/or a nonionic surface activating agent (e.g., Tween, pluronic or polyethylene glycol (PEG)) are preferably included in a dosage it is nontoxic and preferably to be preferably used to a recipient and density.

an appropriate carrier of the illustrations includes a neutral buffered saline or serum albumin and a mixed physiological salt solution. Preferably the product is prescribed as freezing desiccant using an appropriate diluting agent (e.g., sucrose). Other standard carriers, diluent and the diluting agent can be included depending on a wish. The other exemplary constituents include the Tris buffer of pH approximately 7.0-8.5 or the acetic acid buffer of pH approximately 4.0-5.5, and these can include sorbitol or the appropriate alternatives more.

the common method of dispensing of the constituent of the present invention is shown below. Note that it should be noted that it can be produced by well-known method of dispensing about an animal drug constituent, quasi-drugs, a fisheries medicine constituent, a food constituent and the cosmetic composition.

the constituents of the present invention combine with a pharmaceutically acceptable carrier as needed, and it can be administered parenterally. An example of a pharmaceutically acceptable carrier includes agents a diluting agent, a lubricant, a binder, disintegrating agents, a collapse inhibitor, sorbefacient, an absorbent, humecant, a solvent, solubilizer, a suspending agent, tonicity adjusting agents, a buffer, analgesia. Also, the preparation additives such as a preservative, anti-oxidant, a coloring agent, the sweetening agent can be used as needed. Also, a material except NELL-1 can be combined with constituents of the present invention. A vein is intramuscular, and a parenteral administration route includes a subcutaneous administration, intradermal injection, a mucosal administration, the dosage in the rectum, a vaginal administration, local application, the cutis dosage, but is not limited to them. About the preparation of such a pharmaceutically acceptable constituent, as for considering pH, isotonicity, stability, the technique of those skilled in the art is.

even more particularly, the constituent of the present invention may include a coloring agent, preservative, a flavor, a flavoring agent, sweetener and other medicine.

in one situation, the present invention makes it differentiates, and the cell that orientation of constant differentiation was done guided (materials), and materials to do to a more differentiated induced cell, tissue or an organ in way of the differentiation are provided. These materials can include the material which is changed into to function as NELL-1 in a point of A) sustained release anchorage and B) NELL-1 or said differentiation.

in one embodiment, the controlled-release anchorage as used herein can be an extracellular matrix. For example, an extracellular matrix includes collagen, atelocollagen, but is not limited to these. Because, with the present invention, the controlled-release anchorage is because it is preferable if NELL-1 can be provided to a target and a cell doing persistently. The collagen is, for example, available from white Arabian bird sales agency Shiromizu trade Co., Ltd. Co., Ltd. (Osaka-shi), and the collagen from other sources of supply is available in the present invention in turn. In other preferred embodiments, it is understood that any preferred form like what is described in above composition in the present specification can be adopted.

(A Kit)

in other situations, the present invention makes it differentiates, and the cell that orientation of constant differentiation was done guided, and a kit to do to a more differentiated induced cell, tissue or an organ in way of the differentiation is provided. The said kit can comprise the material that it is changed to function as NELL-1 at a point of NELL-1 or said differentiation. In other preferred embodiments, it is understood that any preferred form like what is described in above composition, materials, a method in the present specification can be adopted.

(A Medical Device)

in other situations, the present invention makes it differentiates, and the cell that orientation of constant differentiation was done guided, and a medical device to do to a more differentiated induced cell, tissue or an organ in way of the differentiation is provided. This medical device,

a) NELL-1 or the material that it is changed to function as NELL-1 at a point of said differentiation,

B) controlled-release anchorage and

C) container can be comprised. In other preferred embodiments, it is understood that any preferred form like what is described in above composition, materials, a kit in the present specification can be adopted.

(A Production Method)

in another situation, the present invention provides a method to produce a cell, tissue or organs. This method is the following process:

A process to provide the cell that orientation of constant differentiation was accomplished,

A process to make the material which is changed into to function as NELL-1 in the cell that orientation of the said constant differentiation was accomplished and a point in time of NELL-1 or the said retention touch can be included.

in one embodiment, the cell that orientation of the constant differentiation was done can be provided in the production method of the present invention by the environment with a blood vessel maintaining adipose tissue and it.

in another embodiment, by the differentiation induction method of the present invention, can be touched on a controlled-release anchorage with cell and above NELL-1 where orientation of constant differentiation was done, but is not limited to these. Is not restricted by theory, but while NELL-1 acts, the cell that it is NELL-1 and a differentiation derivative target touches, the reason is because it should be in a state. In other preferred embodiments, it is understood that any preferred form like what is described in above composition, materials, a method, a kit in the present specification can be adopted.

there is the product by the production method of the present invention within the present invention, too.

(The Constituent of Retention Applications)

in another situation, the present invention provides a constituent to maintain a differentiation induced cell, tissue or an organ. This constituent can include the material that it is changed to function as NELL-1 at a point of NELL-1 or said retention.

transplanted cell, tissue and the organ maintain form and size, and the constituent of retention applications of the present invention has advantageous effect outside expectation to be able to maintain the function that they further originally have.

in one embodiment, as for the retention of the differentiation induced cell with the constituent of retention applications of the present invention, tissue or the organ, the presence of the said differentiated induced cell, tissue or the organ can be performed in an inadmissible place. For example, a place (e.g., the adipose tissue to the liver tissue piece) where there is not the cell, tissue or organ to the machine body for the place where the presence of a differentiation induced cell, tissue or the organ is inadmissible is included, but is not limited to these. Even if it is formed in vivo or it was guided even if the constituent of retention applications of the present invention was transplanted in an organism, the immunorejection to them is inhibited, and it is invivo, and it can be maintained. Even if transplanted cell, tissue and the organ were natural, and it was guided differentiation when it was transplanted in an organism, it may have been guided differentiation artificially.

in a preferred embodiment, for example, the constituent of retention applications of the present invention can maintain a liver, kidney, pancreas, an adrenal capsule, a thyroid gland, an ovary mammal, spermary, but is not limited to these.

it is thought that there is generally the etiology that it is not permitted the presence that transplanted organs are ectopic for foreign body recognition/a reject mechanism as referred to as the rejection. Here, in NELL-1, that there were the organization potency of the hematopoietic system cell, organization potency of heterotopic germ-layer tissue was found by the present invention. Is not restricted by theory, but, by NELL-1, the self-organizing that does not receive exclusion by immunomechanism was formed newly, or it may be the formation middle that a transplanted organ is not refused when these findings are considered.

a cell, tissue maintained by a constituent of the present invention or the organ has a function to cope with to exist naturally. About these functions, an example is enumerated below. A liver:

1) a metabolic function: A protein-producing function sugar and fat content are saved as energy, and to be conducted

2) a detoxication function: A function it metabolizes, and to make toxic substance non-toxicity

3) an excretion function: Biliary secretory functions particularly, a hepatic function can be ensured by measuring the elevation of albumin-producing and the hepatic cell function marker (tryptophan oxygenase).

Pancreas:

By a pancreas beta cell inner, a pancreatic function can be ensured by measuring the rise of the yields such as secreted insulin and the precursor

An Adrenal Capsule:

By a thing in confirmation of the steroid hormone secretion, an adrenal function can be confirmed

A Thyroid Gland:

By a thing in confirmation of the secretion of the thyroid hormone (triiodothyronine) and thyroxine), a thyroid function can be confirmed

An Ovary Mammal:

An ovarian function can be confirmed by confirming secretion of the estrogen, progestational secretion

spermary: A testoid function can be ensured by performing testosterone, dihydrotestosterone, secretion confirmation of dehydro epi-androst Ron kidney:

In another embodiment that can ensure a nephr-function by performing secretion confirmation of the erythropoietin, quantity of NELL⁻1 included in the constituent of retention applications of the present invention can be about 0.01 μg/ml-approximately 1,000 μg/mL, about 0.1 μg/ml-approximately 100 μg/mL preferably about 5 μg/ml-approximately 50 μg/mL, about 5 μg/ml-approximately 10 μg/mL more preferably approximately 5 μg/mL more than approximately 0.01 μg/mL. For example, the quantitative lower limit of this NELL-1 can be approximately 0.01 μg/mL, approximately 0.1 μg/mL, approximately 0.2 μg/mL, approximately 0.3 μg/mL, approximately 0.4 μg/mL, approximately 0.5 μg/mL, approximately 0.6 μg/mL, approximately 0.7 μg/mL, approximately 0.8 μg/mL, approximately 0.9 μg/mL, approximately 1.0 μg/mL, approximately 1.5 μg/mL, approximately 2.0 μg/mL, approximately 2.5 μg/mL, approximately 3.0 μg/mL, approximately 3.5 μg/mL, approximately 4.0 μg/mL, approximately 4.5 μg/mL, approximately 5.0 μg/mL, approximately 5.5 μg/mL, approximately 6.0 μg/mL, approximately 6.5 μg/mL, approximately 7.5 μg/mL, approximately 8.0 μg/mL, approximately 8.5 μg/mL, approximately 9.0 μg/mL, approximately 9.5 μg/mL, arbitrary numerical value of about 0.01 μg/ml-approximately 1,000 μg/mL including approximately 10.0 μg/mL. For example, the quantitative upper limit of this NELL-1 can be approximately 10.0 μg/mL, approximately 9.5 μg/mL, approximately 9.0 μg/mL, approximately 8.5 μg/mL, approximately 8.0 μg/mL, approximately 7.5 μg/mL, approximately 6.5 μg/mL, approximately 6.0 μg/mL, approximately 5.5 μg/mL, approximately 5.0 μg/mL, approximately 4.5 μg/mL, approximately 4.0 μg/mL, approximately 3.5 μg/mL, approximately 3.0 μg/mL, approximately 2.5 μg/mL, approximately 2.0 μg/mL, approximately 1.5 μg/mL, approximately 1.0 μg/mL, approximately 0.9 μg/mL, approximately 0.8 μg/mL, approximately 0.7 μg/mL, approximately 0.6 μg/mL, approximately 0.5 μg/mL, approximately 0.4 μg/mL, approximately 0.3 μg/mL, approximately 0.2 μg/mL, approximately 0.1 μg/mL, arbitrary numerical value of about 0.01 μg/ml-approximately 1,000 μg/mL including approximately 0.01 μg/mL. Even if this numerical value is put in the case of (cell (e.g., neutrophils, the eosinocyte of the hematopoietic system, basophils, leucocyte such as the lymphocyte, an erythrocyte, blood platelet, macrophage), an epithelium pro-exocrine gland (glandular system, e.g., appendant in a perspiratory gland, sebaceous glands, an intestinal gland, a gastric gland,) such as salivary glands, hair, bulbus pili, hair-root, hair-root) such as a transplanted organ (e.g., testicular a liver, kidney, pancreas, an adrenal capsule, a thyroid gland, an ovary mammal) and an organ formed in an organism, it is similar. Those skilled in the art can put the quantity of NELL-1 to incorporate into a constituent of the present invention together for the applications, and it can be decided appropriately, and the density can be determined definitely.

in other preferred embodiments, it is understood that any preferred form like what is described in above composition, materials, a method, a kit in the present specification can be adopted.

(A Retention Method)

in another situation, the present invention provides a method to maintain a differentiation induced cell, tissue or an organ. The process that this method provides the said differentiation induced cell, tissue or an organ,

A process to give the material which is changed into to function as NELL-1 in a point in time of NELL-1 or the said retention to the said differentiation induced cell, tissue or an organ can be included. In other preferred embodiments, it is understood that any preferred form like what is described in above composition, materials, a method, a kit in the present specification can be adopted.

(Use)

in another situation, as for the present invention, orientation of constant differentiation provides the use of the material which is changed into by a done cell to function as NELL-1 in a point in time of NELL-1 in the medical and pharmaceutical production to form or the said formation with a differentiation induced cell, tissue or an organ.

in another situation, the present invention provides the use of the material which is changed into to function as NELL-1 in a point in time of NELL-1 in the medical and pharmaceutical production to maintain a differentiation induced cell, tissue or an organ or the said retention.

in other preferred embodiments, it is understood that any preferred form like what is described in above composition, materials, a method, a kit in the present specification can be adopted.

the whole refers to the references such as scientific literature cited herein, a patent, the patent application in the present specification to degree same as what was each described specifically, and it is quoted.

a preferred embodiment was shown for simpleness of the understanding, and the present invention was described as things mentioned above. The present invention is described below based on an embodiment, but above-mentioned explanation and the following embodiments were provided only by the object of the illustration, and it was not provided for the purpose of limiting the present invention. Thus, the range of the present invention is limited to both an embodiment described in the present specification specifically and an embodiment only by, but should not be limited to, claims.

### EXAMPLES

the present invention is described below with an embodiment more specifically, but the present invention is not limited to these embodiments. In an embodiment, as for the used tests, NELL-1 used the thing which Katayama chemical industry Co., Ltd. obtained from the production unless otherwise specified from the common test maker. The animal experiment was based on ethic rule prescribed in Showa University. After when the experiment to the person is performed, obtaining informed consent, it is conducted.

(A Preparation of NELL-1)

as NELL-1, hNELL1 protein was produced and was refined, and it was used for the following embodiments.

for a yield of the hNELL1 protein and purification, it is sigma Aldrich Japan Co., Ltd., GE healthcare bioscience (old Amersham bioscience Co., Ltd.), in vitro Gen Co., Ltd., Funakoshi Co., Ltd., Cosmo bio Co., Ltd., Japan bio Ladd, Kia gene Co., Ltd., Promega Co., Ltd., Takara bio Co., Ltd., Toyobo Co., Ltd., Daiichi Pure Chemicals Co., Ltd., Greiner Japan Co., Ltd., DS Phrmabiomedical (previously known as Dainippon Pharmaceutical laboratory products part Co., Ltd.), Japan Beckton Dickinson Co., Ltd., Merck Co., Ltd., Hydra chemistry Co., Ltd., parkin Elmer life science Japan Co., Ltd., Japan Millipore Co., Ltd., Japan Waters Co., Ltd., Tosoh Co., Ltd., G Elsa Jens Co., Ltd., Shiseido Co., Ltd., Thailand technical center Co., Ltd., Affymetrix Co., Ltd., as one Co., Ltd., Tokyo Glass Kikai Co., Ltd., IKA Japan Ltd., parkin Elmer Japan Co., Ltd., Roche die Agnos tick Co., Ltd., oriental Saccharomyces cerevisiae Co., Ltd., medicine creature research institute Co., Ltd., immunity creature research institute Co., Ltd., peptide research institute Co., Ltd., Toyobo Engineering Co., Ltd., Sanko Junyaku stocks It was purchased from company, Berri TASS Co., Ltd., tef grass co-company, colander avian mortar Co., Ltd., Seikagaku Kogyo Co., Ltd., the Kurabo Industries, Ltd. biomedical part, or an available test was used.

in accordance with exemplary embodiments, a yield of the hNELL1 protein and purification were performed.

(An Isolation of hNELLcDNA)

by a method to describe below, 2448-bphNELL1cDNA was isolated from a human brain cDNA library by PCR first.

(Organization of the Plasmid)

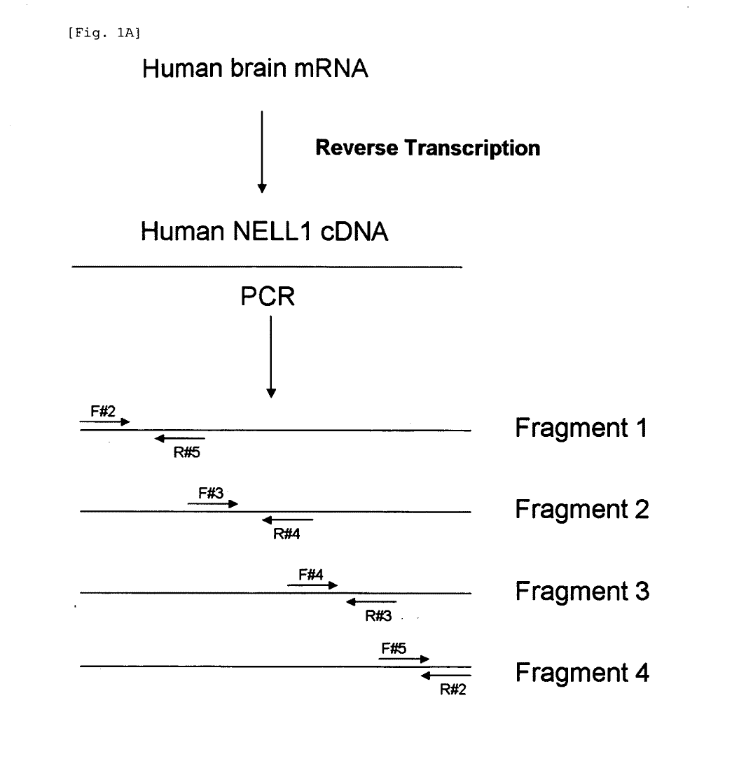

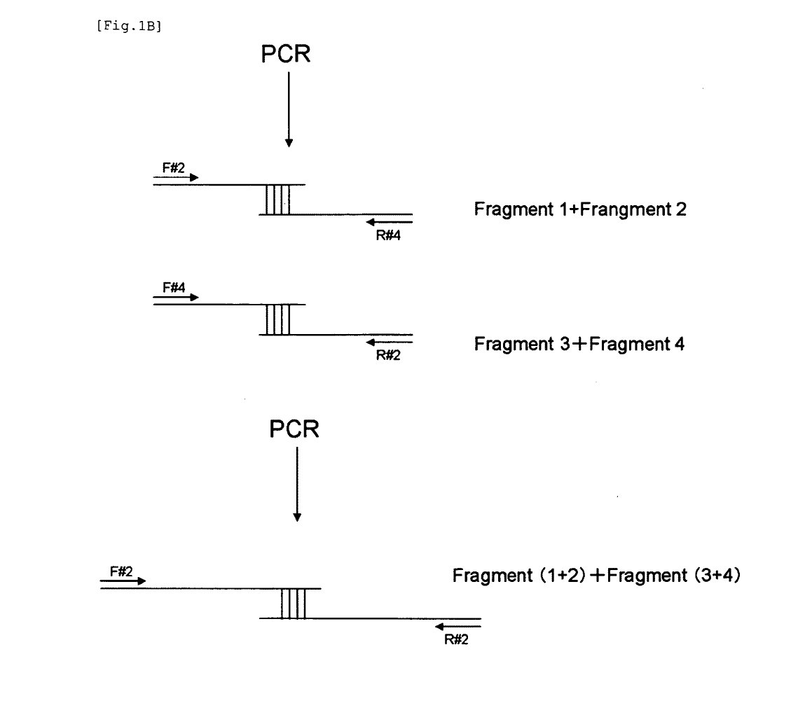

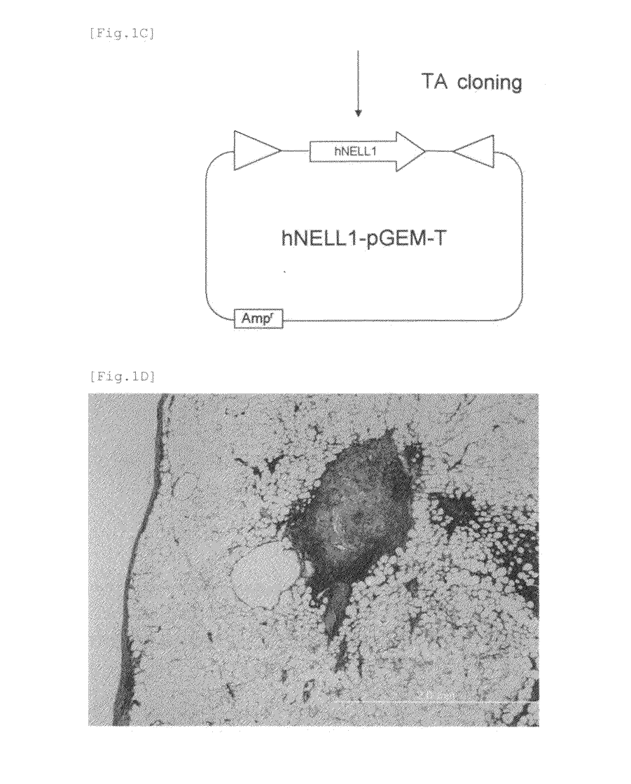

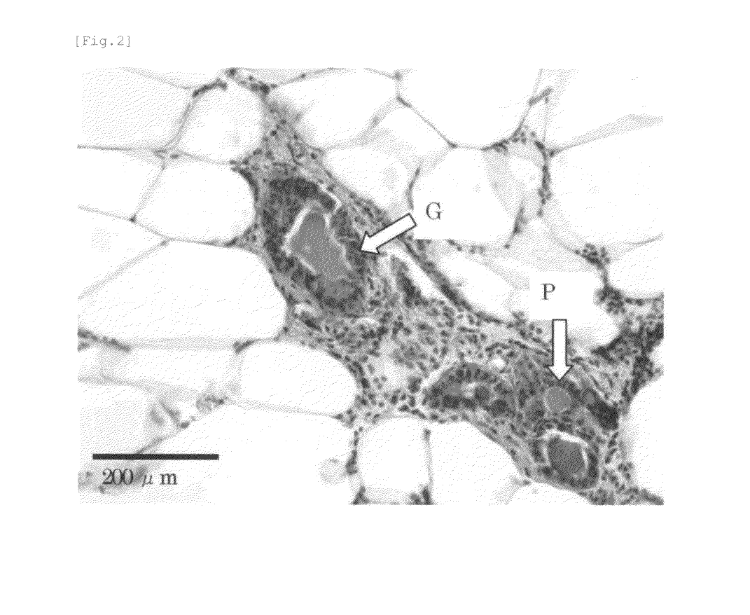

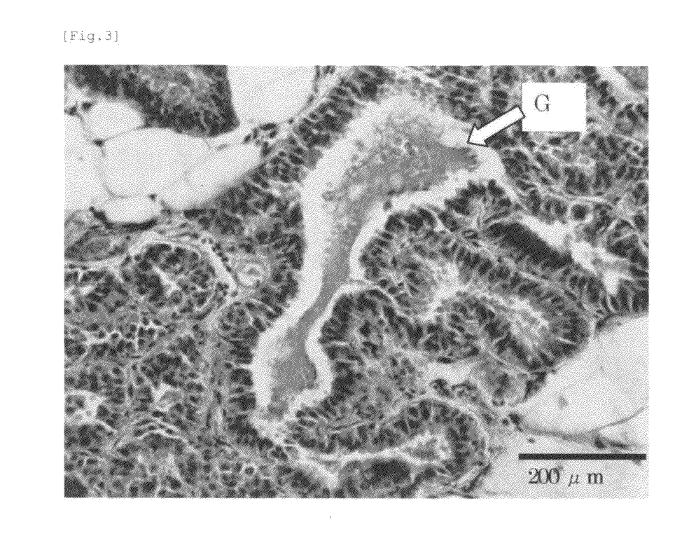

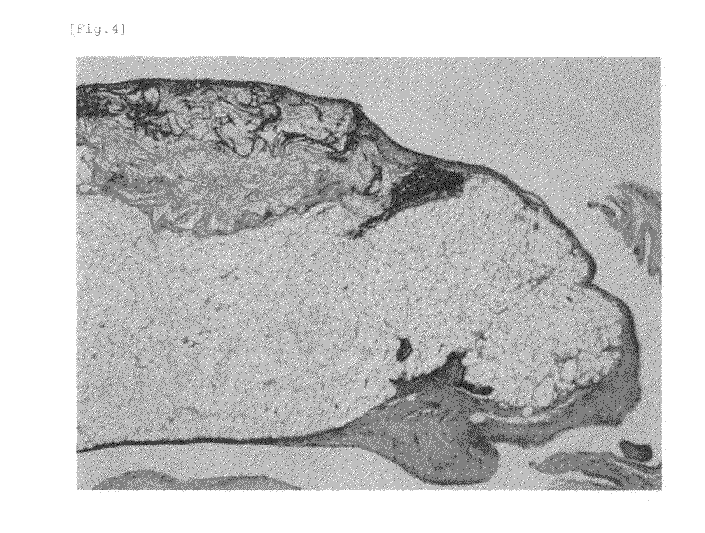

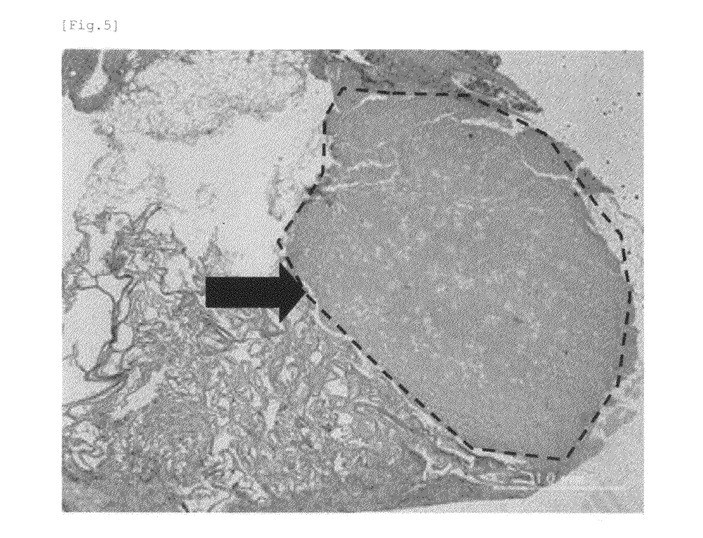

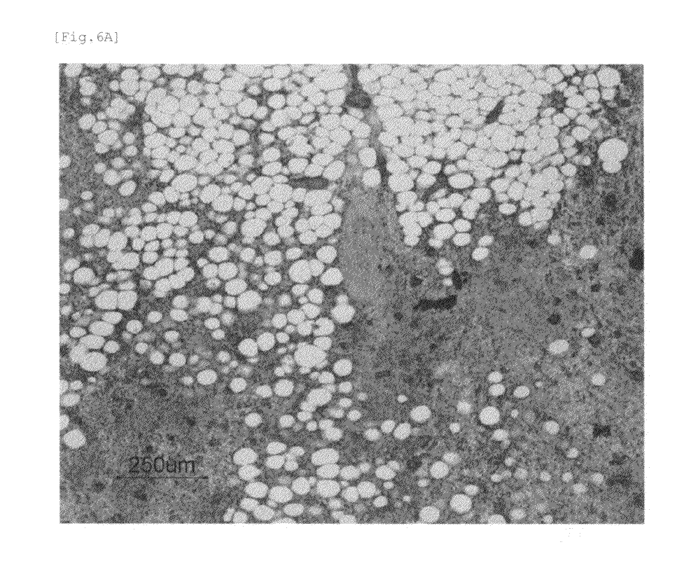

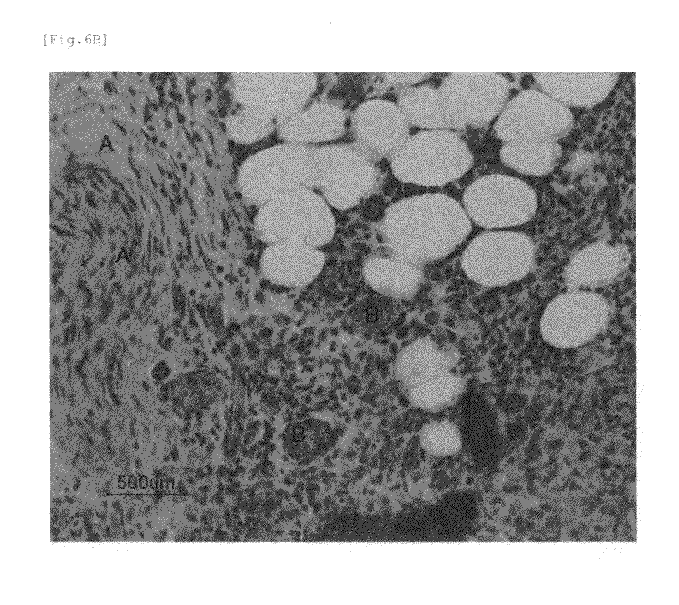

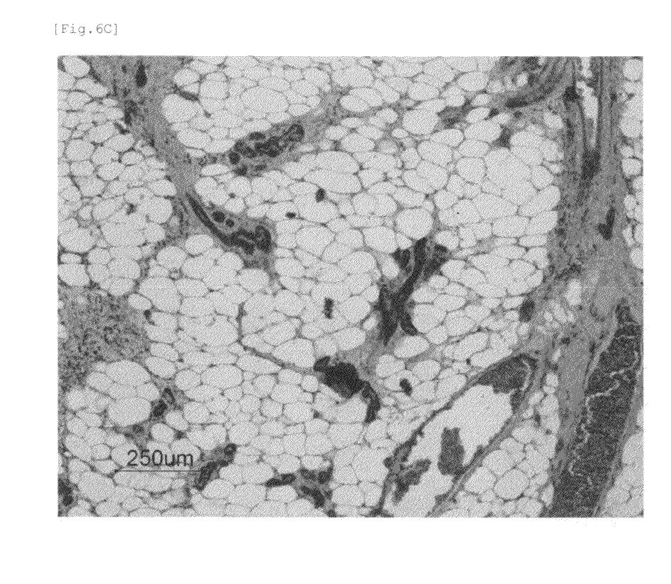

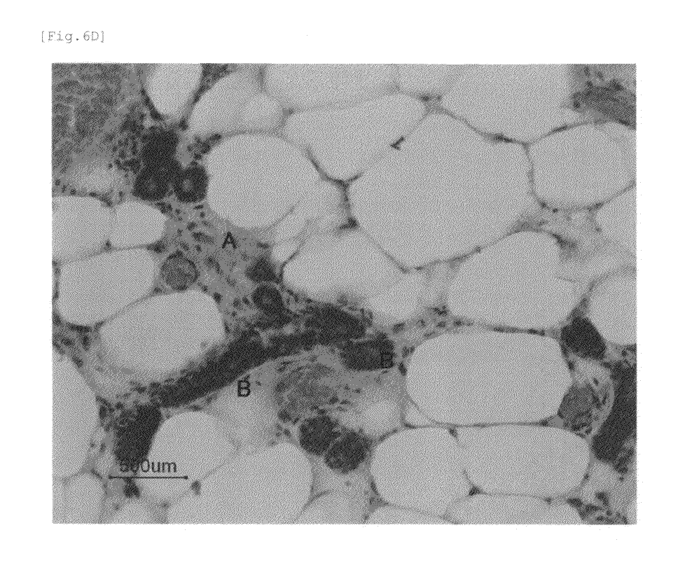

showing architectural synopses of the plasmid (FIGS. 1A-1C).

(1) human brain cDNA was prepared using reverse transcription reaction from whole human brain RNA (HumanbrainwholeRNA).

(2) four fragments were manufactured using each primer than line in order to prepare provided Homo sapiens brain cDNA by a preparation by the PCR method in human NELL-1cDNA.

(3) linking respective two fragments, two new fragments were further manufactured by the PCR method in the fragment of provided four.

(4) further connected two provided fragments to one of them by the PCR method, and full length was obtained.

(5) humanNELL1 plasmid was manufactured by a TA cloning to a vector for pGEM-T clonings at provided full length.

(A Detailed Protocol)

human NELL1cDNA was made by reverse transcription from human brain mRNA.

humanbrainwholeRNA (2.5 mg/mL, Clontech) of 2 μL and Oligo (dT) of 1.5 μL 12-18 (Invitrogen) was mixed as well as the 1.5 mL tube which diethylpyrocarbonate handling of 13.5 μL ultrapure water (DEPC-UPW) was poured into, and it was left at rest in room temperature for 20 minutes. Then it was saved at −20 degrees Celsius after 10× PCR buffer (GIBCO) of 3 μL, 25 mMMgCl2 of 1.5 μL, 0.1MDTT of 3 μL, 25 mMdNTP of 2 μL might be added to this tube, and it was mixed, and a spin was downed, and, in addition, reverse transcription reacted at 42 degrees Celsius for 60 minutes, and having heated SuperscriptII (Invitrogen) at 72 degrees Celsius 1.5 μL for 15 minutes.

then, four following fragments 1-4 (f1-4) was bet on PCR, and it was amplified.

the primer who used 1 (f1) 2 (f2) 3 (f3) 4 (f4) fragment 527 bp fragment 624 bp fragment 612 bp fragment 841 bp and the reaction liquid are as follows.

TABLE 1

| Primer | | |
|---|---|---|
| No. | Sequence, 5′-3′ | Position |
| F#2 | GGAAGCTTCGGAGCGATGCCGATG | 87-120 |
| | GATTTGATT (SEQ ID NO: 19) | |
| F#3 | TCAGTTAGCGCCTCTCATCTC | 564-584 |
| | (SEQ ID NO: 20) | |
| F#4 | GCGGATTTTAACCAAGAGCTG | 1139-1159 |
| | (SEQ ID NO: 21) | |
| F#5 | GTGTCTGTCCATCTGGATTCAC | 1705-1726 |
| | (SEQ ID NO: 22) | |
| R#2 | GTAACCGGTTCAATTATTTTGAAGAC | 2544-2508 |
| | ACTCAAAATCC (SEQ ID NO: 23) | |
| R#3 | ATCTTTCTCGCAGTGGCTTCC | 1749-1729 |
| | (SEQ ID NO: 24) | |
| R#4 | CTAAAACTCCACCTCGGCATT | 1185-1165 |
| | (SEQ ID NO: 25) | |
| R#5 | ATCCTGTTACAGTCGACATGG | 610-590 |
| | (SEQ ID NO: 26) | |

| | | |
|---|---|---|
| Plasmid DNA (10 times attenuation) | 1 | μL |
| 10* Thermopol. A buffer solution | 5 | μL |
| 10 mMdNTP (SIGMA) | 1 | μL |
| A 10 μL primer (forward direction) | 1 | μL |
| A 10 μL primer (opposite direction) | 1 | μL |
| VENTDNApol. (NEB) | 1 | μL |
| Ultrapure water (UPW) | 40 | μL |
| The total | In 50 | μL |

f1, in forward primer F #2 (SEQ ID NO: 19) and reverse primer R #5 (SEQ ID NO: 26), f2, in forward primer F #3 (SEQ ID NO: 20) and opposite direction primer R #4 (SEQ ID NO: 25), f3, forward direction primer F #4 (SEQ ID NO: 21) and opposite direction primer R #3 (SEQ ID NO: 24) and f4 used a combination of forward direction primer F #5 (SEQ ID NO: 22) and opposite direction primer R #2 (SEQ ID NO: 23).

Subsequently 55 degrees Celsius 30 seconds and 72 degrees Celsius one minute were processed 25 cycles after processing repeatedly for 72 degrees Celsius seven minutes for 95 degrees Celsius 30 seconds for 95 degrees Celsius two minutes, and PCR reaction was maintained at 4 degrees Celsius.

Then, the following reaction liquid was prepared to tie f1 and f2 or f3 and f4, and PCR reaction was performed.

| PCR reaction liquid (f1 and f2 or f3 and f4) | For each 1 | μL |
|---|---|---|
| 10* Pfu buffer solution | 5 | μL |
| 10 mMdNTP (SIGMA) | 2 | μL |
| 10 μM primer (orthodromic)# 2 or # 4) | 1 | μL |
| 10 μM primer (retrograde)# 4 or # 2) | 1 | μL |
| PfuturboDNApol. (STRATAGENE) | 1 | μL |
| Ultrapure water (UPW) | 38 | μL |

50 μL in total

For 60 degrees Celsius 1 and 72 degrees Celsius dichotomy were repeated 3 cycles for 95 degrees Celsius 1 after processing PCR reaction for 95 degrees Celsius 2. Then, a primer was added to reaction liquid. Even more particularly, after having repeated for 55 degrees Celsius 1 and 72 degrees Celsius dichotomy 27 cycles for 95 degrees Celsius 1, it was reacted for 72 degrees Celsius 10, and it was maintained to 4 degrees Celsius.

Even more particularly, PCR was performed two fragments that f1 and f2 or f3 and f4 were tied, and manufactured were tied, and to obtain Homo sapiens NELL1cDNA full length. Reaction liquid was prepared as follows.

| PCR reaction liquid (f1-2, f3-4) | For each 1 | μL |
|---|---|---|
| 10* Pfu buffer solution | 5 | μL |
| 10 mMdNTP (SIGMA) | 2 | μL |
| 10 μM primer (orthodromic)# 2) | 1 | μL |
| 10 μM primer (retrograde)# 2) | 1μ | |
| LPfuturboDNApol. (STRATAGENE) | 1 | μL |
| Ultrapure water (UPW) | 38 | μL |

50μ in total

The LPCR reaction repeated for 60 degrees Celsius 1 and 72 degrees Celsius dichotomy 3 cycles for 95 degrees Celsius 1 after processing for 95 degrees Celsius 2. Then, a primer (it refers to the following list) was added to reaction liquid. Even more particularly, after having repeated for 55 degrees Celsius 1 and 72 degrees Celsius dichotomy 27 cycles for 95 degrees Celsius 1, it was reacted for 72 degrees Celsius 10, and it was maintained to 4 degrees Celsius. DpnI (NEB) was added to this reaction liquid 1 μL, and it was reacted at 37 degrees Celsius for one hour. Ethanol precipitation was performed of provided reaction liquid, and precipitation was dissolved in ultrapure water (UPW). 10 mMdATP and 10*TaqDNA polymerase buffer of 0.5 μL were added 2.5 μL. TaqDNA polymerase was added 0.5 μL, and it was reacted for 72 degrees Celsius [symbol] 20 minutes.

In addition, ultrapure water (UPW) was done with 10 μL in full length humanNELL11 μL, T4DNALigase 1 μL provided than pGEM-T vector 1 μL, 2× Rapid Ligation Buffer 5 μL, PCR in a 0.5 mL tube, and a TA cloning did a provided nucleic acid by a one-hour incubated thing in room temperature.

The following lists are information used for a primer design.

TABLE 2
Primer candidate

| hNELL1 | forward #1: | 51-GGCTCATTTGCTTCCACCTAG-31 (21-mer) (SEQ ID NO: 30) | |
|---|---|---|---|
| | forward #2: | 51-GGAAGCTTCGAGAGCGATGCCGATGGATTTGATT-31 (34-mer) | |
| | | (SEQ ID NO: 19) | |
| | forward #3: | 51-TCAGTTAGCGCCTCTCATCTC-31 (21-mer) (SEQ ID NO: 20) | #564-#584 |
| Tm: | 64 GC: 52.38 | | |
| | forward #4: | confirming primer | #1139-#1159 |
| | forward #5: | 51-gtgtctgtccatctggattcac-31 (21-mer) (SEQ ID NO: 22) | #1705-#1726 |
| | reverse #1: | 52-GTCATTTCGTCCATTCTTCTG-31 (21-mer) (SEQ ID NO: 31) | |
| | reverse #2: | 51-GTAACCGGTTCAATTATTTTGAAGACACTCAAAATCC-31 (37-mer) | |
| | | (SEQ ID NO: 23) | |
| | reverse #3: | confirming primer | #1749-#1729 |
| | reverse #4: | 51-CTAAAACTCCACCTCGGCATT-32 (21-mer) (SEQ ID NO: 25) | #1185-#1165 |
| Tm: | 62 GC: 47.62 | | |
| | reverse #5: | 51-atcctgttacagtcgacatgg-31 (21-mer) (SEQ ID NO: 26) | #610-#590 |

| | |
|---|---|
| Fragment size | f2-r5: 527 bp (f1) |
| | f3-r4: 624 bp (f2) |
| | f4-r3: 612 bp (f3) |
| | f5-r2: 841 bp (f4) |

(A Yield of the hNELL1 Protein and Purification)

(Synopses)

The hNELL1cDNA fragment used a thing provided by this enforcement.

hNELL1cDNA fragment was inserted into the down stream of the OpIE2 promoter of expression vector pIZT/V5-His included in InsectSelectGlowSystem (Invitrogen, Carlsbad, Calif.), and C-extremity V5- and *6-His tag produced form (hNELL1-VH). Then, using FuGene6 (Roche, Mannheim, Germany), it was transfected in provided plasmid pIZT/V5-His-NELL1, and Trichoplusiani-derivedHighFive™ cell (Invitrogen) was incubated in ExpressFiveSFM (Invitrogen) which was a serum-free culture medium for 48 hours. Antibiotics Zeocin (NakalaiTesque, Kyoto, Japan) was added to a culture medium at 400 μg/mL, and Zeocin resistant cell was selected by changing a culture medium every 3-4 days. To monitor an extracellular yield of the hNELL1-VE protein, culture medium (6 mL) antiV5 tag antibody (1 μg) It was incubated with NakalaiTesque), and 6% of sediment was offered in the sodiumdodecyl sulfate-polyacrylamide gel electrophoresis (SDS-PAGE) of the gel and the Western blotting which used above-described antibody on a PVDF film. Because it was large, and affinity purified hNELL1-VE protein, wrote to the plastic column (Φ 1.5*10 cm) which filled NiSepharose6FastFlow (GEHealthcare UK, Buckinghamshire, UK), and culture medium (to 500 mL) of the third day was washed enough with a phosphoric acid buffered saline (PBS), and it was eluted in PBS 500 mM imidazole. It was dialyzed to PBS at 4 degrees Celsius enough overnight and this effluent (approximately 10mL) was concentrated to approximately 900 μg/mL by ultrafiltration. Degree of purity of the hNELL1-VH protein was confirmed by staining using SDS-PAGE and CoomassieBrilliantBlueR-250. More particularly, it is as follows.

(Materials and a Method)

(An Expression Vector)

It was synthesized than human brain mRNA, and the insect cells expression vector was built using human NELL1 (hNELL1) cDNA (ORF2433 bp) which performed a cloning. The expression vector used pIZT/V5-His (in vitro Gen Corporation). SpeI of the multicloning site in a vector and SacII were used to insert hNell1. After having amplified a DNA fragment to insert by the PCR method, it was cut with the restriction enzyme, and it was ligated. Also, about the anastomosis tag expression vector, stop codon of hNELL1 was used as for the non-expression vector of the anastomosis tag except stop codon of hNELL1. The genetic information and the protein information were retrieved than a database in the Internet.

Genetic Information NCBI

It is db=nucleotide & val=1827482 http//www.ncbi.nlm.nih.gov/entrez/viewer.fcgi

Protein Information

Swiss-Prot

It is //au.expasy.org/uniprot/Q92832 http

A tag portion: PRFEGKPIPNPLLGLDSTRTGHHHHHH (SEQ ID NO: 29)

Among tag portions, the SacII cloning site part is PR.

Among tag portions, the V5 tag portion is equivalent to a nucleic acid encoding PIPNPLLGLDST (SEQ ID NO: 27).

Among tag portions, the histidine tag portion copes with a nucleic acid encoding HHHHHH (SEQ ID NO: 28). Thereafter, stop codon enters.

(An Establishment of the Transduction Cell Strain)

(A Cell and a Culture Medium)

The insect cells used HiFive (in vitro Gen Corporation), and the culture medium used the thing which added 200 mML-glutamine 90 mL and appropriate antibiotics (penicillin and streptomycin) in ExpressFiveSFM (in vitro Gen Corporation #10,486-0251L).

(Transduction)

The transduction used act, Gene pulsar (bioLadd Corporation) by electroporation. It made DNA10 mg and a cell (1×106 unit) were added, and the cuvet (bioLadd Corporation) of the 4 mm in width slit suspend lightly, and 125 μF, a pulse of 300V were provided after Hikami cooling for ten minutes. Then return culture was performed to a culture medium after cooling in 10-minute ice.

(Expression Cell Selection (Zeocin Resistance))

This vector added zeocin to a culture medium to have resistance in zeocin (in vitro Gen Corporation) so that it was with 0.5 mg/mL of final concentration, and culture was repeated twice for three days. Also, this vector ensured the cell which was transformed in an epi illumination-style fluorescence microscope bottom (CKX41 Olympus Corporation) by the thing in confirmation of the GFP expression because GFP was coexpressed.

(A Little Culture)

After the cell which showed zeocin resistance was assumed an expression strain, and having sowed to a 10 cm dish (BD Falcon) in 20% confluency, it was cultured on 4th, and conditioned media was collected by a high speed centrifugal separation (for 5000 rpm20). Note that the culture was conducted at 27 degrees.

(Protein Purification)

Nickel sepharose FF6 carrier (Amersham bioscience company) was used for purification. After Econocolumn (bioLadd Corporation) was filled with carrier 1 mL, and having equilibrated in an equilibration buffer (10 mMTris-HCl, 20 mMImidazole, 150 mMNaCl pH 7.6), centrifugal separation treated conditioned media was drained into the direct column, and affinity by the gravity-drop was connected. Then it was eluted in an elution buffer (10 mMTris-HCl, Imidazole, 0.5 mMPMSF, pH 7.6) after having washed in an irrigation buffer (10 mMTris-HCl, 20mMImidazole, 150 mMNaCl pH 7.6). It is done the solid layer, and, as for the nickel sepharose, Nitrilotriacetiacid (NTA) can maintain a nickel ion using NTA and symphysis of nickel on the sepharose surface. Also, only protein fusing with nickel with a His tag using a binding with the histidine can be purified. It was substituted with supplemented object protein histidine by using the cheap imidazole which resembled histidine in a conformation (it refers to the following chemical formula.), and the elution isolated objective protein.

[chemical formula 1]

[symbol]

A dialysis tube (a product name: Spectra/Por2, a model number: 132680: Spectrum Laboratories) was cut to length approximately 10 cm, and it was boiled for five minutes, and it was washed. A tube after the boiling was washed with pure water, and entered, and rim one end closed the NELL-1 protein solution which one end was closed by an exclusive clip, and was collected by a clip while outrunning air. A pontoon was attached to the tube, and it was dipped into the PBS (−) of 5 L that was a dialysis external solution, and it was stirred at 4 degrees Celsius calmly. It continued being dialyzed hourly while changing a dialysis external solution four times. After a dialysis end, the solution in the tube was collected. After recovery, it was filtered by a φ 0.22 μm filter (a product name: Mirex-GV filter unit, a model number: SLGV033RB: Japan Millipore), and it was assumed preparation NELL-1 protein.

20* PBS (-)

| | |
|---|---|
| NaCl | 480 g |
| NaHPO4/12H2O | 174 g |
| KCl | 12 g |
| KH2PO4 | 12 g |

PBS (-) which improved a female to 1 L with ultrapure water
20* PBS (-) was diluted to 20 times with ultrapure water.]

### The Formation of the Example 1 Hematopoietic System Tissue

(The Manufacture of the Silicon Mold)

Silicone rubber impression material (vinyl poly siloxane impression material Exafine management charges instrument 23BZ0035, G C den Tal products Co., Ltd., Tokyo, Japan) was used to manufacture silicon mold. Columnar Teflon (a registered trademark) magnet bar (1-4206-07) (4 mm in diameter, 8 mm in length, as one Co., Ltd., Tokyo, Japan) was covered with the film of the silicone rubber. After silicon stiffened, a Teflon (a registered trademark) magnet bar was removed by cutting into half of the length. A slim dimple was made in a part cut in half from one end of the silicon mold to secure the space for vascular flaps.

The silicon mold can make the thing which was able to be matched with grafting organ form. For example, the thing of the rat bone tip configuration and a pine nut-shaped thing using the clay are manufactured, and it can be used.

(The Manufacture of the Incubator in the Organism)

An intraperitoneal administration was performed of pentobarbital (0.1 ml/100 gw) after transduction with rat (rat male 8 weeks of age of Wistar origin, 25 of them, purchase: Saitama animal used for experiment) ether.

Skin incision was added to a rat femoral region, and the neurohemal bunch comprising a lower abdominal wall status pulse to diverge from a thigh status pulse and side-by-side travel nerves was exfoliated from a surrounding tissue.

Adipose tissue of the abdominal regions subcutis was separated to the size of the silicon mold mainly on the furcation of the lower abdominal wall status pulse in total, and a lower abdominal wall status pulse was done with a blood vessel handle and the caulescent flap which did.

Collagen sponge (copter stat white Arabian bird product cord 4300010 high managed care instrument 20700BZG00024000 Co., Ltd.) which dropped NELL-1 protein (5 μg/mL, 10 μg/mL or 50 μg/mL) in a silicon mold (opposite sides) was inserted. It was sewed up in 5-0 nylon threads (Hasegawa medial BRAK-K15A05H), and an adeps distal end was fixed to the one side of the silicon mold. It covered with a remaining silicon mold, and lateral two places were sewed up in 5-0 nylon threads (Hasegawa medial BRAK-K15A05H), and it was fixed.

(The Manufacture of the Pocket to the Femoral Line Interval and Grafting)

It made sure of the locus which could be buried in a silicon mold intramuscularly without a vascular handle coming under excessive pressure, and, in addition, the pocket which reached the buttocks cutis was manufactured in abrasion of becoming dull in rat femoral region muscle. Specifically, it exfoliated between adductor muscle and gracilis in the inferior limb reckoning, and a pocket was manufactured intramuscularly.

It was buried in the pocket which manufactured silicon mold. Then, the entry port of the pocket was sewed up using 5-0 nylon threads (Hasegawa medial BRAK-K15A05H), and it was closed down. It was confirmed that a vascular peduncular blood flow was not obstructed, and a shut wound did a femoral region.

A rat was anesthetized using diethyl ether from grafting two weeks later or four weeks later, and a grafting area was resected. 10% of extracted areas were fixed with neutral holm Arde Homo sapiens solution, and paraffin preparation was manufactured. Slicing thinly was performed of the preparation in a 5 μm section, and hematoxylin-eosin (HE) was dyed, and it was observed with a light microscope in bright field.

(Formed Histionic Functional Confirmation)

It carries out following experiments to confirm that tissue formed in the present embodiment functions as a hematopoietic system organization.

That a hematopoietic system tissue functions in vivo is ensured by examining the expression of the cell marker of the hematopoietic system by immunochemistry staining and furo-site met Lee.

By immunochemistry staining, a localization in the tissue of the cellular marker of the hematopoietic system is labelled for three dimensions, and it can examine by a thing in confirmation of the histology.

In furo-site met Lee, a cell is extracted by measuring cell numbers by the landmark to the target cell, a cell marker, and a disjunction can examine a cell according to various differentiation stages.

(A Fructification)

In a grafting area (the environment with adipose tissue and a blood vessel maintaining it), a hematopoietic system tissue was able to be derived by devising an addition method of the NELL-1 protein

(FIG. 1D). NELL-1 dedifferentiates fat-cell, and it is conceivable even if it made a hematopoietic system cell redifferentiate. However, because a blood flow is maintained with the adipose tissue in the silicon mold, under NELL-1 presence, the possibility that existing hematopoietic stem-cell (an undifferentiated somatic stem cell) differentiates in peripheral blood more, and was guided is considered. Also, the possibility that a stem cell derived from the adipose tissue which there is to adipose tissue differentiated to a hematopoietic system cell is considered.

In the incubator in the organism, the thing which plural clumping of the hematopoietic system tissue were distributed over was recognized. In these, an unfinished blood corpuscle (leucocyte) was ensured by histology, and the blood vessel which commuted with a connected blood vessel was found at the same time.

### Histogenesis when Comparative Example 1 BMP, TGF β, FGF were Used

An invivo incubator is manufactured using a method like Example 1 except that BMP, TGF β, FGF are used in this comparative example in substitution for NELL-1. The incubator in the organism which added NELL-1 is manufactured without including the incubator in the organism which added only a buffer solution as negative control (1) and (2) a collagen sponge. The incubator in these organisms is ported like Example 1, and it can examine whether a hematopoietic system tissue is formed.

### The Formation of the Example 2A Glandular System

(The Manufacture of the Incubator in the Organism)

By a method like Example 1, the incubator in the organism was manufactured. Quantity of NELL-1 protein added in a silicon mold (opposite sides) is 5 μg/mL, 10 μg/mL or 50 μg/mL.

(An Albino Rat Used for an Experiment)

The albino rat used rat male 8 weeks of age of Wistar origin, 25 of them (purchase: Saitama animal used for experiment.).

(The Manufacture of the Pocket to the Femoral Line Interval)

Using a method like Example 1, a pocket was manufactured between rat femoral lines, and the incubator in the organism was transplanted in a manufactured pocket. 2-4 weeks after grafting, a histionic configuration formed in the grafting area was ensured by hematoxylin eosin staining.

(Formed Histionic Functional Confirmation)

It carries out following experiments to confirm that tissue formed in the present embodiment functions as a glandular system.

A Perspiratory Gland:

Confirmation of secreted material such as sodium and the apocrine secretion (apocrine secretion)

Sebaceous Glands:

Secretory confirmation such as the lipid

An Intestinal Gland:

Secretory confirmation such as ion peptidase P., the maltase

A Gastric Gland:

Confirmation such as the pepsinogen such as digestive enzymes or a proton or the chloride

A salivary gland: Secretory confirmation of amylase, ptyalin or the peroxidase.

(A Fructification)

In the environment with adipose tissue and the blood vessel which maintained it, an epithelium system cell was able to be guided in the adipose tissue of the caulescent flap by devising an addition method of the NELL-1 protein

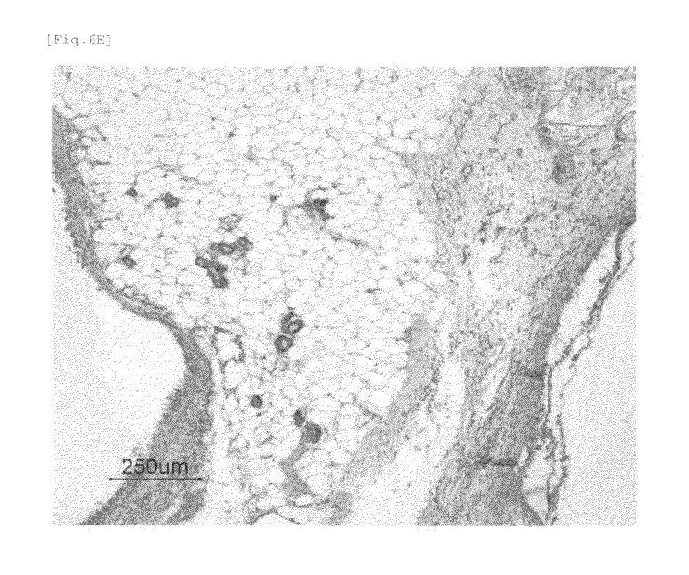

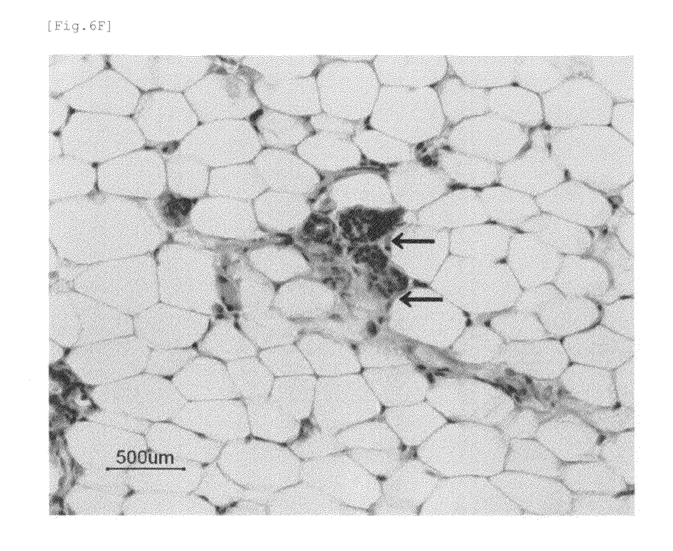

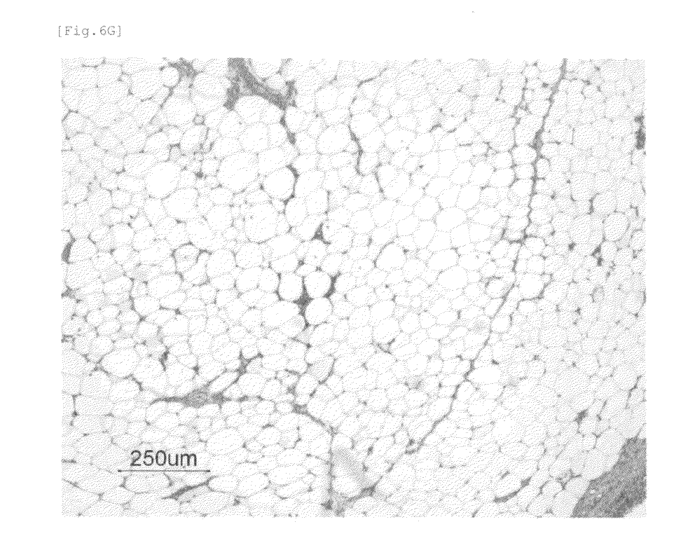

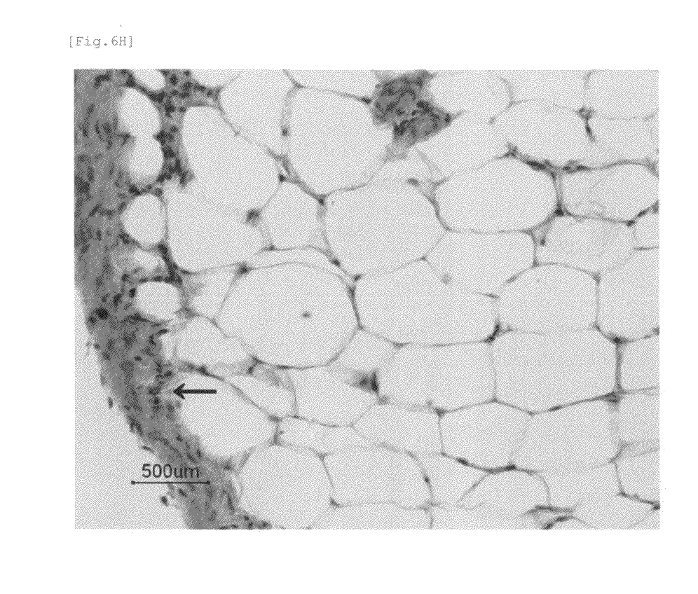

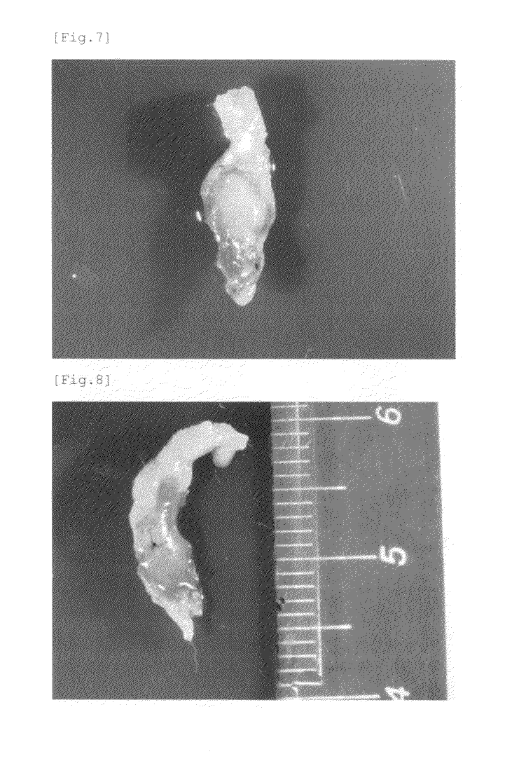

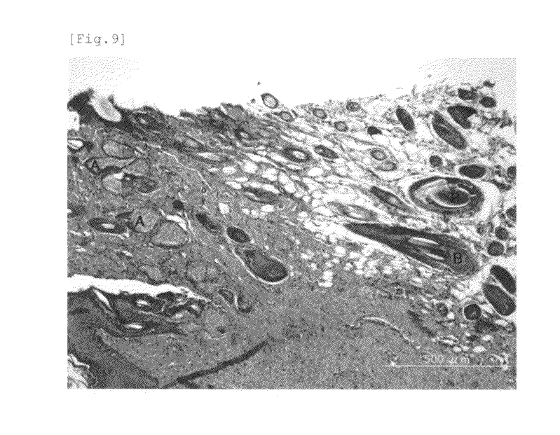

(FIGS. 2 and 3). NELL-1 dedifferentiates fat-cell, and it is conceivable that it makes epithelial cells redifferentiate. However, the adipose tissue is of mesoderm origin, and it is difficult to differentiate to ectodermal epithelial tissue. Because a blood flow is maintained with the adipose tissue in the silicon mold, the cell which already received orientation of the differentiation existing in peripheral blood integrates in limited part under NELL-1 presence, and it is thought that it differentiated in the glandular system which is an epithelium system cell. The epithelial tissue formed newly was an exocrine gland and the pipe

(FIGS. 2 and 3). Because a secretory elaboration and the pipe which transported the secreted material outside a body were ensured, it may be said that formation/retention was able to functionalize epithelium system tissue in adipose tissue in the present embodiment.

Thus, in the cluster which added NELL-1, an epithelial glandular system was ensured by the adipose tissue inside. Even more particularly, it may be said that epithelial (epidermis) composition including the hair-root was confirmed.

Because, as for this, hair-root and the glandular system whole that adipose tissue is connective tissue (mesoblastic genetically) are epithelial tissue (developmental ectoderm), two kinds of inoculation course is possible. 5mm square thickness approximately 2mm is cut out of the back of a fur cut open to perform the present embodiment based on this when it is hair-root or a glandular system accompanying hair it, and an above procedure can be implemented by what is disseminated. Also, that the somatic stem cell corresponding to the hematopoietic stem cell or adipose tissue (mesoblastic genetically) differentiates to epithelial system tissue can be examined. The epithelial system composition can be implemented by what it is cut out of the skin back, and is disseminated.

### Histogenesis when Comparative Example 2 BMP, TGF β, FGF were Used

An invivo incubator is manufactured using a method like Example 2 except that BMP, TGF β are used in this comparative example in substitution for NELL-1. As negative control,

(1) The incubator in the organism which added only a buffer solution and

(2) The incubator in the organism which added NELL-1 is prepared without including collagen sponge. The incubator in these organisms is ported like Example 1, and it can consider whether a glandular system is formed.

### Influence by Example 2B FGF (Fibroblast Growth Factor)

In accordance with exemplary embodiments, the incubator in an albino rat like Example 2A and the organism was used.

(1) The incubator in the organism which added only FGF,

(2) The incubator in the organism which added NELL-1 and FGF,

(3) The incubator in the organism which added only NELL-1 and

(4) The incubator in the organism which added only a physiological salt solution was manufactured. These were transplanted in a rat femoral intermuscular pocket. Two weeks of the grafting later, a grafting area was extracted, and, according to reduction to a single unit, a hematoxylin eosin staining specimen was manufactured, and it was observed.

As a result,

(1) Connective tissue and a blood vessel were formed of the cluster of the incubator in the organism which added only FGF, but the adipose tissue was not formed (FIGS. 6A and B).

(2) In a group of incubators in the organism which added NELL-1 and FGF, the development of the glandular system was ensured two weeks after grafting (FIGS. 6C and D). Arbores more a plurality of than the case that added only NELL1 were seen, and, as for this glandular system, connective tissue covered the glandular system circumference. It was known that the FGF activated hypodermal tissue when it was administered to subcuticular corium, and it followed that induced hypodermal tissue (a glandular system) formation was promoted more by NELL-1 in the incubator in the organism.

(3) In a group of incubators in the organism which added only NELL-1, a glandular system was observed by two weeks later of the grafting, but there was little formation of the connective tissue (FIGS. 6E and F). Four weeks of the grafting later, glandular histology developed more.

(4) In a group of incubators in the organism which added only a physiological salt solution, connective tissue was ensured with adipose tissue circumferentially (FIGS. 6G and H). Note that the connective tissue was observed in all clusters by this adipose tissue people.

### Formation of Example 3 Hair and the Bulbus Pili

(The Manufacture of the Incubator in the Organism)

By a method like Example 1, the incubator in the organism was manufactured. Quantity of NELL-1 protein added in a silicon mold (opposite sides) is 5 μg/mL, 10 μg/mL or 50 μg/mL.

(An Albino Rat Used for an Experiment)

The albino rat used rat male 8 weeks of age of Wistar origin, 25 of them (purchase: Saitama animal used for experiment.).

(Collection of the Hair Including the Hair-Root and Dissemination)

5 mm square thickness approximately 2 mm was cut out of the back of a fur cut open, and hair including the hair-root and epidermis were gathered to an albino rat of Wistar origin. This was disseminated on the collagen sponge of the incubator in the organism.

(Manufacture of the Pocket to the A. Femoral Region Line Interval and Grafting (Autograft))

Using a method like Example 1, a pocket was manufactured between rat femoral lines, and the incubator in the organism which manufactured in the embodiment was transplanted.

2-4 weeks after grafting, a histionic configuration formed in the grafting area was ensured by hematoxylin eosin staining.

(Formed Histionic Functional Confirmation)

It carries out following experiments to ensure that tissue formed in the present embodiment functions as tissue creating hair.

The state of the grafting area was ensured by the observation due to the unaided eye, tissue staining by the tissue staining (hematoxylin eosin staining).

(A Fructification)

In the environment with adipose tissue and the blood vessel which maintained it, hair and bulbus pili were able to be guided in adipose tissue by devising an addition method of the NELL-1 protein (FIG. 9). NELL-1 dedifferentiates fat-cell, and it is conceivable even if it made hair and bulbus pili redifferentiate. However, the adipose tissue is of mesoderm origin, and it is difficult to differentiate to the hair which is ectodermal epithelium system tissue and bulbus pili. To transplanted root of hair, a glandular system (a perspiratory gland) which accompanied hair, bulbus pili, hair-root and hair-root was ensured. It may be said that formation/retention functionalized hair in adipose tissue in the present embodiment than the above.

### Histogenesis when Comparative Example 3 BMP, TGF β, FGF were Used

An invivo incubator is manufactured using a method like Example 3 except that BMP, TGF β, FGF are used in this comparative example in substitution for NELL-1. The incubator in the organism which added NELL-1 is prepared without including the incubator in the organism which added only a buffer solution as negative control (1) and (2) a collagen sheet. The incubator in these organisms is ported like Example 1, and it can examine whether hair and bulbus pili are formed.

### Example 4

### Liver Tissue Single Grafting

(The Manufacture of the Incubator in the Organism)

By a method like Example 1, the incubator in the organism was manufactured. Quantity of NELL-1 protein added in a silicon mold (opposite sides) is 5 μg/mL, 10 μg/mL or 50 μg/mL.

(An Albino Rat Used for an Experiment)

The albino rat used rat male 8 weeks of age of Wistar origin, 25 of them (purchase: Saitama animal used for experiment.).

(The Collection of the Liver Tissue Piece and Dissemination)

In an animal same as the albino rat which ported the incubator in the organism, a processus xiphoideus part was cut open 1 cm in length, and 1 cm was removed surgically from a liver (a left lobe) tip. The liver piece after the ablation was divided 3-5 (3 mm every direction) under a physiology salt, and, by what was disseminated in a silicon mold, a liver piece was put in the environment with the blood vessel which maintained adipose tissue and it. Hemostasis was confirmed after an ablation, a suture shut wound was made later.

(Grafting)

The incubator in an organism made in the embodiment was ported in a pocket manufactured by a method like Example 1. In doing so, it was careful so that the bloodstream of the caulescent flap did not stop.

A rat was anesthetized using diethyl ether from grafting two weeks later or four weeks later, and a grafting area was resected. 10% of extracted areas were fixed with neutral holm Arde Homo sapiens solution, and paraffin preparation was manufactured. Slicing thinly was performed of the preparation in a 5 μm section, and hematoxylin-eosin (HE) was dyed, and it was observed with a light microscope in bright field.

(The Functional Confirmation of a Transplanted Tissue Piece)

It carries out following experiments to demonstrate that ported composition keeps the function as the liver. Because a pipe and an afferent canal are essential for the function reproduction of the organ that the efferent canals except blood vessels such as the large-sized organ or kidney are necessary, it is examined mainly on an endocrine system.

The hepatic function is classified roughly into following three:

1) A metabolic function: Sugar and fat content are saved as energy, and protein is produced

2) A detoxication function: It metabolizes with toxic substance, and detoxication

3) An excretion function: Secretion there biliary, these can be examined whether a liver functions in vivo by measuring the elevation of albumin-producing and the hepatic cell function marker (tryptophan oxygenase) in particular.

(A Fructification)

As for the liver piece which ported NELL-1 in 5 μg of addition group, lobulus hepatis form was maintained (FIG. 5).

in the environment with adipose tissue and the blood vessel which maintained it, the presence that was ectopic by an application with NELL-1 protein in vivo could maintain an inadmissible liver tissue piece with a liver tissue piece.

(B. Another House Grafting)

From rat male 8 weeks of age of Wistar origin, a liver tissue piece was gathered by a method like the present embodiment. Then, it was similar, and it was transplanted like the present embodiment in the albino rat of the other individual piece. As a result, the liver piece transplanted was maintained.

### Influence when a Comparative Example 4A Physiological Salt Solution was Used

A method like Example 4 was used except that a physiological salt solution was used in the) book comparative example in substitution for NELL-1.

As a result, in the group which added a physiological salt solution, as for the liver piece which transplanted, only a residue after fat histology, the macrophage which absorbed the liver piece which transplanted and an absorption/the decomposition was recognized in grafting four weeks later without being ensured (FIG. 4: a control group).

(A Summary)

Life arrived to the liver tissue piece four weeks after grafting when self or an another family transplanted a gathered liver tissue piece in the incubator in an organism made by the rat abdomen with NELL-1. The growth was not recognized to this tissue piece.

It necrotized or, in the control group which added a physiological salt solution in substitution for NELL-1, the liver tissue piece was taken in. It is thought that the combination of incubator and NELL-1 in this organism may act as “an in vivo incubator”. There is not yet the report on the method that is effective for a cell, tissue, the grafting of the organ till now. Without the process of the present invention causing immunorejection about a cell, tissue, the grafting of the organ, implant in an organism straight, it is conceivable that it can be with an effective method (e.g., liver transplantation) to make arrive.

### Histogenesis when Comparative Example 4B BMP, TGF β, FGF were Used

Using a method like Example 4, the influence that they give a liver piece can be examined except that BMP, TGF β, FGF are used in this comparative example in substitution for NELL-1.

### Example 5

### Ovary Tissue Single Grafting

(The Manufacture of the Incubator in the Organism)

By a method like Example 1, the incubator in the organism was manufactured. Quantity of NELL-1 protein added in a silicon mold (opposite sides) is 5 μg/mL, 10 μg/mL or 50 μg/mL.

(An Albino Rat Used for an Experiment)

The albino rat used rat male 8 weeks of age of Wistar origin, 25 of them (purchase: Saitama animal used for experiment.).

(Ovarian Histionic Collection)

Abdominal region right and left were incised than a dorsal. After the apertural area was enlarged for a peritoneal cavity than a dorsal, and inclusive ligation did the periovular adipose tissue in oviduct and the oviduct tip, a bilateral ovary mammal was gathered. The ovary mammal picked out was a bead type of approximately 5mm.

(The Inoculation to the Incubator in the Ovarian Histionic Organism)

An ovary mammal was put in the environment with adipose tissue and the blood vessel which maintained it by disseminating an ovary mammal gathered in the embodiment to the incubator in the organism.

(Grafting)

The incubator in an organism made in the embodiment is ported in a pocket manufactured like Example 1. In doing so, it was careful so that a bloodstream did not stop. It is cultured under NELL-1 presence for 12 weeks.

(The Functional Confirmation of a Transplanted Tissue Piece)

It can be ensured whether the composition that was ported by examining estrogen, progestational secretion keeps the function as the ovary mammal.

(A Fructification)

In the target cluster, a remarkable reduction of the bone quantity was seen by experimental osteoporosis by the influence that resected an ovary mammal in the control group (Mayahara M, Anat RecA Discov Mol Cell Evol Biol. 2003 September, 274(1)817-26).

Bone quantity decrease to be angry at after oophorectomy by measuring a sponge bone mass under the epiphyseal plate such as rat femurs used for an experiment by μCT for three dimensions can be ensured whether the incubator in the organism which disseminated an ovary mammal was able to be inhibited.

### Histogenesis when Comparative Example 5 BMP, TGF β, FGF were Used

A method like Example 5 is used except that BMP, TGF β, FGF are used in this comparative example in substitution for NELL-1. As negative control, only a buffer solution is added. These can be examined whether Example 5 and a similarly ovarian tissue piece are maintained.

### Example 6

### Pancreas, Adrenal Capsule, Thyroid Gland and Testoid Tissue Single Grafting

(Tissue Collection Protocol)

In accordance with exemplary embodiments, each tissue piece is gathered using an albino rat like Example 4.

(Pancreas)

An abdominal region median section is added, and ventriculus is removed above. A tissue piece of the size of the approximately 5mm corner is gathered from a pancreas ensured by the gastric backside.

(An Adrenal Capsule)

An abdominal region median section is added, and exclusion does a small intestine, duodenum. A tissue piece of the size of the approximately 5mm corner is gathered from a nephr-adrenal capsule confirmed above.

(A Thyroid Gland)

Thyroid one side attaching on either side of tracheal cartilage ensured under a cervical median section, a muscular coat is gathered.

(Spermary)

An abdominal region median section is added, and an epithelium is reversed towards spermary from the pelvic circumference, and a testis is extracted.

(Grafting)

Each tissue piece was put in the environment with the blood vessel which maintained adipose tissue and it by adding each tissue piece gathered in the embodiment to an invivo incubator including NELL-1 using a method like Example 4. These are transplanted in a pocket manufactured by a method like Example 1. After grafting, it can be confirmed whether each tissue is maintained by the observation due to the unaided eye, histologic observation.

(The Functional Confirmation of a Transplanted Tissue Piece)

Pancreas: By a pancreas beta cell inner, a pancreatic function can be ensured by measuring the rise of the yields such as secreted insulin and the precursor,

An Adrenal Capsule:

By a thing in confirmation of the steroid hormone secretion, an adrenal function can be confirmed,

A Thyroid Gland:

By a thing in confirmation of the secretion of the thyroid hormone (triiodothyronine) and thyroxine), a thyroid function can be confirmed,

Spermary:

A testoid function can be ensured by performing testosterone, dihydrotestosterone, secretion confirmation of dehydro epi-androst Ron,

Kidney: A nephr-function can be confirmed by performing secretion confirmation of the erythropoietin.

### Histogenesis when Comparative Example 6 BMP, TGF β, FGF were Used

A method like Example 6 is used except that BMP, TGF β, FGF are used in this comparative example in substitution for NELL-1. As negative control, only a buffer solution is added. These can be examined whether a pancreas, an adrenal capsule, a thyroid gland and a testoid tissue piece are maintained with Example 5 similarly.

### The Influence that Example 7 NELL-1 Provides to a Cell

In accordance with exemplary embodiments, the influence that NELL-1 gives to a cell is retrieved. As a cell, a stem cell derived from adipose tissue, hematopoietic stem-cell, a mesenchyme system stem cell, an undifferentiated cell included in an organ piece, a somatic stem cell, fat-cell are used. These cells are prepared so that it is described in the following bibliographies or it is obtained.

A stem cell derived from adipose tissue: It is implications for cell-based therapies. Tissue Eng. 2001 April, 7(2)211-28 Zuk P A, Multilineage cells from human adipose tissue.

A hematopoietic stem-cell and mesenchyme system stem cell:

Alison M R, Hepatocytes from non-hepatic adult stem cells., Nature. 2000 Jul. 20, 406 (6793) 257:

The undifferentiated cell which was included in an organ piece: It is Transplantation. 2007 May 27, 83(10)1337-40 Ogawa K, Living donor liver transplantation with reduced monosegments for neonates and small infants.

In accordance with exemplary embodiments, epithelial tissue, a cell included in the adipose tissue are obtained, and the influence that ENLL-1 gives them equally can be examined.

NELL-1 is added by various kinds of density (e.g., 5 μg/mL, 10 μg/mL, 50 μg/mL) to these cells, and NELL-1 can observe influence to give each cell.

### Histogenesis when Comparative Example 7 BMP, TGF β, FGF were Used

Using a method like Example 7, influence to give each cell can be examined except that BMP, TGF β, FGF are used in this comparative example in substitution for NELL-1.

The Grafting to Organisms such as the Tissue which Embodiment 8 was Formed

In accordance with exemplary embodiments, tissue formed in embodiment 1-3 can be examined whether it is transplanted, and tissue transplanted by observing course such as the tissue is maintained in vivo by an animal whether the function that natural corresponding tissue originally has is run.

### The Induction of the Example 9 Liver Cell and Retention

In accordance with exemplary embodiments, a liver cell is guided from a stem cell using hepatocytegrowthfactor (HGF) which is a hepatocyte growth factor, dexamethasone, oncostatin. The preparation of the liver growth factor is conducted based on Kamiya A, OncostatinM and hepatocyte growth factor induce hepatic maturation via distinct signaling pathways. FEBS Lett. 2001 Mar. 9, 492(1-2)90-4. A liver cell is disseminated to an invivo incubator,

1) A metabolic function (a protein-producing function sugar and fat content are saved as energy, and to be conducted),

2) A detoxication function (a function it metabolizes, and to make toxic substance non-toxicity),

3) A hepatic function can be ensured by measuring an excretion function (biliary secretory functions) particularly the rise of albumin-producing and the hepatic cell function marker (tryptophan oxygenase).

### The Induction of the Example 10 Pancreas Beta Cell and Retention

In accordance with exemplary embodiments, a pancreas beta cell is derived from a stem cell using beta cell phosphorus, this feh phosphorus. The preparations such as beta cell phosphorus, this feh phosphorus perform Kojima I, Conophylline based on a novel differentiation inducer for pancreatic beta cells. Int J Bio chem Cell Biol. 2006, 38(5-6):923-30.

A culture beta cell is disseminated to an invivo incubator, by a pancreas beta cell inner, a pancreatic function can be ensured by measuring the rise of the yields such as secreted insulin and the precursor.

### A Comparison with the Technique of Example 11 Isogai

Technique (Noritaka Isogai, Hirohisa Kusuhara, Yoshito Ikada, Hitoshi Ohtani, Robin Jacquet, Jennifer Hillyer, Elizabeth Lowder, William J. Landis. Tissue Engineering. Apr. 1, 2006, 12 (4): 691-703.doi: 10.1089/ten.2006.12.691.) of Isogai is performed, and the this matter method is compared.

### The Manufacture of the Incubator in the Organism Including an Example 12 Atelocollagen Gel and Grafting

The silicon mold used a thing manufactured by a method like Example 1.

(Adjustment/Injection Method 1 mL of the Atelocollagen Gel)

High managed care instrument authorization number 16100BZZ01355000 was purchased, and the atelocollagen gel was used.

Using the PBS which was finished with sterilization, it was diluted so that NELL-1 became 500 μg/mL and 50 μg/mL. NELL-1 dilution was added for two collagen gel injection barrel wound packages (3%) finished with sterilization, and two glass syringes were connected, and around 15 times of push-out was mixed alternately. In use, the NELL-1 dose became 50 μg and 5 μg to administer 0.1 mL.

(The Manufacture of the Organism Incubator)

Instead of using the collagen sponge which dropped NELL-1 protein, using a method like Example 1, an organism incubator was manufactured except that atelocollagen gel adjusted in the embodiment was used.

(The Formation of the A. Hematopoietic System Tissue)

A pocket was manufactured between femoral lines like Example 1, and an organism incubator was ported. A grafting area was extracted from grafting two weeks later or four weeks later, and paraffin preparation was manufactured, and hematoxylin eosin staining was performed. As a result, the formation of the hematopoietic system tissue was confirmed.

(The Formation of the B. Glandular System)

A pocket was manufactured between femoral lines like Example 2, and an organism incubator was ported. A grafting area was extracted from grafting two weeks later or four weeks later, and paraffin preparation was manufactured, and hematoxylin eosin staining was performed. As a result, the formation of the glandular system was confirmed.

(The Formation of the C. Glandular System)

In accordance with exemplary embodiments, instead of using the collagen sponge which dropped NELL-1 protein, an organism incubator was manufactured using a method like Example 3 except that atelocollagen gel adjusted in the embodiment was used. Hair including the hair-root or epidermis was gathered like Example 3.

Hair including gathered hair-root or epidermis was disseminated in an atelocollagen gel. A pocket was manufactured between femoral lines like Example 3, and an organism incubator was ported. A grafting area was extracted from grafting two weeks later or four weeks later, and paraffin preparation was manufactured, and hematoxylin eosin staining was performed. As a result, the formation of the glandular system (a perspiratory gland) which accompanied hair, bulbus pili, hair-root and hair-root was confirmed.

(The Retention of the D. Liver Tissue Piece)

When a liver tissue piece was transplanted to an invivo incubator like Example 4, the transplanted liver tissue piece was maintained.

### The Experiment by Injecting Example 13 NELL -1 Continually

In accordance with exemplary embodiments, the incubator in the organism which did not include a controlled-release anchorage in collagen sponge, an atelocollagen gel was used. The incubator in an organism used in the present embodiment was manufactured by a method like Example 1 except that it was not soaked with a controlled-release anchorage.

In the incubator in the organism which did not add NELL-1, there was [symbol] with 30 G needle (it is a common needle), and dilute NELL-1 was administered with a physiological salt solution. As for the dosage, implant did a needle for the mold which was already buried by the rat abdominal region under the ether anesthesia, and inside sinus was recognized from the hardness in the mold of the incubator in the organism, and it was administered.

An examination to inject a stain solution (a mosquito is latched [symbol] No, 30,020-2, Muto chemistry) diluted to purple 50 times before extraction was performed to confirm that the dosage of NELL-1 by this method was certain, and that the dosage of NELL-1 was certain was ensured (FIGS. 7 and 8).

NELL-1 in the mold was able to be kept high concentration as well as intraoperative once by this injection method by administering NELL-1 frequently.

In accordance with exemplary embodiments, 20 μg/mL in total was administered (NELL1 solution 0.1 mL of 50 μg/1 ml) every other week for four weeks. Then a grafting area was extracted using a method like Example 1, and a paraffin specimen was manufactured, and hematoxylin eosin staining was performed, and histology was ensured. As a result, a hematopoietic system tissue or a glandular system was ensured. This is an effect like time using the collagen sponge.

Also, an appendant glandular system (a perspiratory gland) was ensured by hair, bulbus pili, hair-root and hair-root when hair including the hair-root and the epidermis were transplanted to an invivo incubator like Example 3.

As for each histionic formation, the presence or absence of controlled-release anchorage was able to ensure what tissue formed by doing the density in the mold of NELL-1 constantly than these fructifications without influencing.

Even more particularly, the transplanted liver tissue piece was maintained when a liver tissue piece was transplanted to an invivo incubator like Example 4.

### The Manufacture of the Incubator Invivo in the Environment with Example 14 Muscular Tissue and a Blood Vessel Maintaining it and Grafting

In accordance with exemplary embodiments, in substitution for adipose tissue and the caulescent flap including the lower abdominal wall status pulse, the caulescent flap including the blood vessel which maintained muscular tissue and muscular tissue was used. The technique except it manufactured an invivo incubator (an invivo incubator including the collagen sponge, the incubator in the organism including the atelocollagen gel and the incubator in the organism which does not include a controlled-release anchorage) using a method like Example 1, Example 12 and 13. When the incubator in the organism which did not include a controlled-release anchorage was ported, NELL-1 was injected like Example 13 from the outside the body.

(The Formation of the A. Hematopoietic System Tissue)

A pocket was manufactured between femoral lines like Example 1, and each organism incubator was ported. A grafting area was extracted from grafting two weeks later or four weeks later, and paraffin preparation was manufactured, and hematoxylin eosin staining was performed. As a result, the formation of the hematopoietic system tissue was confirmed.

(The Formation of the B. Glandular System)

A pocket was manufactured between femoral lines like Example 2, and each organism incubator was ported. A grafting area was extracted from grafting two weeks later or four weeks later, and paraffin preparation was manufactured, and hematoxylin eosin staining was performed. As a result, the formation of the glandular system was confirmed.

(The Formation of the C. Glandular System)

In accordance with exemplary embodiments, instead of using the collagen sponge which dropped NELL-1 protein, using a method like Example 3, each organism incubator was manufactured except that atelocollagen gel adjusted in the embodiment was used. Hair including the hair-root or epidermis was gathered like Example 3.

Hair including gathered hair-root or epidermis was disseminated in an atelocollagen gel. A pocket was manufactured between femoral lines like Example 3, and an organism incubator was ported. A grafting area was extracted from grafting two weeks later or four weeks later, and paraffin preparation was manufactured, and hematoxylin eosin staining was performed. As a result, the formation of the glandular system (a perspiratory gland) which accompanied hair, bulbus pili, hair-root and hair-root was confirmed.

(The Retention of the D. Liver Tissue Piece)

When a liver tissue piece was transplanted to an invivo incubator like Example 4, the transplanted liver tissue piece was maintained.

### INDUSTRIAL APPLICABILITY

The present invention has a utility to provide formation and/or a technique to maintain with a differentiation induced cell, tissue and an organ.

The present invention provides a utility to be able to protect a differentiation induced cell, tissue transplanted in an organism and an organ from immunorejection.

### SEQUENCE LISTING

PRC6D6_{—}3.txt

1. A composition for inducing differentiation of a cell having been directed to a given differentiation, to a further differentiated cell, tissue or organ directed to the given differentiation, wherein the composition comprises NELL-1 or a substance which is altered to function as NELL-1 at the time of said inducing differentiation.

2. The composition according to claim 1, wherein the composition is for heterotopically forming said further differentiated cell, tissue or organ directed to the given differentiation in the presence of fat tissue and blood vessels wherein the blood vessels support the fat tissue.

3. The composition according to claim 1, wherein said cell having been directed to a given differentiation is a somatic stem cell.

4. The composition according to claim 3, wherein said somatic stem cell is a somatic stem cell present in a tissue selected from the group consisting of hematopoietic tissue, epithelial tissue, connective tissue, muscle tissue and nerve tissue.

5. The composition according to claim 4, wherein said somatic stem cell is a stem cell having been directed to differentiation of hematopoietic tissue, epithelial tissue or connective tissue.

6. The composition according to claim 5, wherein the somatic stem cell present in the hematopoietic tissue is a hematopoietic stem cell, which is a mother cell of a blood cell having self repair capability and multipotency.

7. The composition according to claim 5, wherein the somatic stem cell present in the epithelial tissue is a stem cell having been directed to differentiation of epithelial tissue, which is a mother cell of epithelial system cell having self repair capability and multipotency.

8. The composition according to claim 5, wherein the somatic stem cell present in the epithelial cell is adenoblast or a cell included in a hair root.

9. The composition according to claim 1, wherein the differentiated cell, tissue or organ is differentiated hematopoietically or epithelially.

10. The composition according to claim 9, wherein the hematopoietically differentiated cell, tissue or organ is a blood cell differentiated from hematopoietic stem cell, selected from the group consisting of a leukocyte selected from the group consisting of neutrophil, eosinophil, basophil and lymphocyte; erythrocyte; platelet; macrophage; and a combination thereof.

11. The composition according to claim 9, wherein the epithelially differentiated cell, tissue or organ is exocrine gland and a duct thereof

12. (canceled)

13. The composition according to claim 9, wherein the epithelially differentiated cell, tissue or organ is hair, hair bulb, hair root or a gland tissue associated with hair root.

14. The composition according to claim 1, wherein said cell having been directed to a given differentiation is adenoblast, and said differentiated cell, tissue or organ is exocrine gland and a duct thereof.

15-17. (canceled)

18. The composition according to claim 1, wherein said differentiated cell, tissue or organ has a function corresponding to that present in nature.

19. A material for inducing differentiation of a cell having been directed to a given differentiation, to a further differentiated cell, tissue or organ directed to the given differentiation, said material comprises:
A) a sustained-releasing scaffold; and
B) NELL-1 or a substance which is altered to function as NELL-1 at the time of said inducing differentiation.

20-21. (canceled)

22. The material according to claim 21, wherein said sustained-releasing scaffold is selected from the group consisting of collagen and atelocollagen.

23-38. (canceled)

39. A kit for inducing differentiation of a cell having been directed to a given differentiation, to a further differentiated cell, tissue or organ directed to the given differentiation, wherein said kit comprises NELL-1 or a substance which is altered to function as NELL-1 at the time of said inducing differentiation.

40. The kit according to claim 39, wherein the kit is for heterotopically forming a further differentiated cell, tissue or organ directed to the given differentiation in the presence of fat tissue and blood vessels wherein the blood vessels support the fat tissue.

41-45. (canceled)

46. The kit according to claim 39, wherein said NELL-1 is comprised at about 0.01 μg/ml or more.

47. The kit according to claim 46, wherein said NELL-1 is comprised from about 5 μg/ml to about 50 μg/ml.

48-100. (canceled)


SEQUENCE LISTING (31 sequences)

SEQ ID NO: 1 - DNA, 3026 bp, Homo sapiens
  CDS (135)..(2564)
  sig_peptide (135)..(182)
ggcgctgccgagccacctcccccgccgcccgctagcaagtttggcggctccaagccaggcgcgcctcaggatccaggctcatttgcttccacctagcttcggtgccccctgctaggcggggaccctcgagagcgatgccgatggatttgattttagttgtgtggttctgtgtgtgcactgccaggacagtggtgggctttgggatggaccctgaccttcagatggatatcgtcaccgagcttgaccttgtgaacaccacccttggagttgctcaggtgtctggaatgcacaatgccagcaaagcatttttatttcaagacatagaaagagagatccatgcagctcctcatgtgagtgagaaattaattcagctgttccagaacaagagtgaattcaccattttggccactgtacagcagaagccatccacttcaggagtgatactgtccattcgagaactggagcacagctattttgaactggagagcagtggcctgagggatgagattcggtatcactacatacacaatgggaagccaaggacagaggcacttccttaccgcatggcagatggacaatggcacaaggttgcactgtcagttagcgcctctcatctcctgctccatgtcgactgtaacaggatttatgagcgtgtgatagaccctccagataccaaccttcccccaggaatcaatttatggcttggccagcgcaaccaaaagcatggcttattcaaagggatcatccaagatgggaagatcatctttatgccgaatggatatataacacagtgtccaaatctaaatcacacttgcccaacctgcagtgatttcttaagcctggtgcaaggaataatggatttacaagagcttttggccaagatgactgcaaaactaaattatgcagagacaagacttagtcaattggaaaactgtcattgtgagaagacttgtcaagtgagtggactgctctatcgagatcaagactcttgggtagatggtgaccattgcaggaactgcacttgcaaaagtggtgccgtggaatgccgaaggatgtcctgtccccctctcaattgctccccagactccctcccagtgcacattgctggccagtgctgtaaggtctgccgaccaaaatgtatctatggaggaaaagttcttgcagaaggccagcggattttaaccaagagctgtcgggaatgccgaggtggagttttagtaaaaattacagaaatgtgtcctcctttgaactgctcagaaaaggatcacattcttcctgagaatcagtgctgccgtgtctgtagaggtcataacttttgtgcagaaggacctaaatgtggtgaaaactcagagtgcaaaaactggaatacaaaagctacttgtgagtgcaagagtggttacatctctgtccagggagactctgcctactgtgaagatattgatgagtgtgcagctaagatgcattactgtcatgccaatactgtgtgtgtcaaccttcctgggttatatcgctgtgactgtgtcccaggatacattcgtgtggatgacttctcttgtacagaacacgatgaatgtggcagcggccagcacaactgtgatgagaatgccatctgcaccaacactgtccagggacacagctgcacctgcaaaccgggctacgtggggaacgggaccatctgcagagctttctgtgaagagggctgcagatacggtggaacgtgtgtggctcccaacaaatgtgtctgtccatctggattcacaggaagccactgcgagaaagatattgatgaatgttcagagggaatcattgagtgccacaaccattcccgctgcgttaacctgccagggtggtaccactgtgagtgcagaagcggtttccatgacgatgggacctattcactgtccggggagtcctgtattgacattgatgaatgtgccttaagaactcacacctgttggaacgattctgcctgcatcaacctggcagggggttttgactgtctctgcccctctgggccctcctgctctggtgactgtcctcatgaaggggggctgaagcacaatggccaggtgtggaccttgaaagaagacaggtgttctgtctgctcctgcaaggatggcaagatattctgccgacggacagcttgtgattgccagaatccaagtgctgacctattctgttgcccagaatgtgacaccagagtcacaagtcaatgtttagaccaaaatggtcacaagctgtatcgaagtggagacaattggacccatagctgtcagcagtgtcggtgtctggaaggagaggtagattgctggccactcacttgccccaacttgagctgtgagtatacagctatcttagaaggggaatgttgtccccgctgtgtcagtgacccctgcctagctgataacatcacctatgacatcagaaaaacttgcctggacagctatggtgtttcacggcttagtggctcagtgtggacgatggctggatctccctgcacaacctgtaaatgcaagaatggaagagtctgttgttctgtggattttgagtgtcttcaaaataattgaagtatttacagtggactcaacgcagaagaatggacgaaatgaccatccaacgtgattaaggataggaatcggtagtttggtttttttgtttgttttgtttttttaaccacagataattgccaaagtttccacctgaggacggtgtttggaggttgccttttggacctaccactttgctcattcttgctaacctagtctaggtgacctacagtgccgtgcatttaagtcaatggttgttaaaagaagtttcccgtgttgtaaatcatgtttcccttatcagatcatttgcaaatacatttaaatgatctcatggtaaatgttgatgtattttttggtttattttgtgtactaacataatagagagagactcagctccttttatttattttgttgatttatggatcaaattctaaaataaagttgcctgttgtgaaaaaaaaaaaaaaaaaaaaaaaaaaa

SEQ ID NO: 2 - protein, 810 aa, Homo sapiens
MPMDLILVVWFCVCTARTVVGFGMDPDLQMDIVTELDLVNTTLGVAQVSGMHNASKAFLFQDIEREIHAAPHVSEKLIQLFQNKSEFTILATVQQKPSTSGVILSIRELEHSYFELESSGLRDEIRYHYIHNGKPRTEALPYRMADGQWHKVALSVSASHLLLHVDCNRIYERVIDPPDTNLPPGINLWLGQRNQKHGLFKGIIQDGKIIFMPNGYITQCPNLNHTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECSEGIIECHNHSRCVNLPGWYHCECRSGFHDDGTYSLSGESCIDIDECALRTHTCWNDSACINLAGGFDCLCPSGPSCSGDCPHEGGLKHNGQVWTLKEDRCSVCSCKDGKIFCRRTACDCQNPSADLFCCPECDTRVTSQCLDQNGHKLYRSGDNWTHSCQQCRCLEGEVDCWPLTCPNLSCEYTAILEGECCPRCVSDPCLADNITYDIRKTCLDSYGVSRLSGSVWTMAGSPCTTCKCKNGRVCCSVDFECLQNN

SEQ ID NO: 3 - DNA, 2812 bp, Mus musculus
  CDS (40)..(2472)
  sig_peptide (40)..(102)
gcgttggtgcgccctgcttggcggggggcctccggagcgatgccgatggatgtgattttagttttgtggttctgtgtgtgcaccgccaggacagtgctgggctttgggatggaccctgaccttcagatggacatcatcactgaacttgaccttgtgaacaccaccctgggcgtcactcaggtggctggactacacaatgccagtaaggcatttctgtttcaagatgtacagagagagatccactcagcccctcatgtgagtgagaagctgatccagctattccggaataagagtgagtttacctttttggctacagtgcagcagaagccgtccacctcaggggtgatactgtcgatccgggagctggaacacagctattttgaactggagagcagtggcccaagagaagagatacgctatcattacatccatggcggcaagcccaggactgaggcccttccctaccgcatggccgatggacagtggcacaaggtcgcgctgtctgtgagcgcctctcacctcctactccatgtcgactgcaataggatttatgagcgtgtgatagatcctccggagaccaaccttcctccaggaagcaatctatggcttgggcaacgtaatcaaaagcatggctttttcaaaggaatcatccaagatggcaagatcatcttcatgccgaacggcttcatcacacagtgccccaacctaaatcgcacttgcccaacatgcagtgatttcctgagcctggttcaaggaataatggatttgcaagagcttttggccaagatgactgcaaaactgaattatgcagagacgagacttggtcaactggaaaattgccactgtgagaagacctgccaagtgagtgggctgctctacagggaccaagactcctgggtagatggtgacaactgcaggaactgcacatgcaaaagtggtgctgtggagtgccgaaggatgtcctgtcccccactcaactgttccccagactcacttcctgtgcatatttctggccaatgttgtaaagtttgcagaccaaaatgtatctatggaggaaaagttcttgctgagggccagcggattttaaccaagacctgccgggaatgtcgaggtggagtcttggtaaaaatcacagaagcttgccctcctttgaactgctcagagaaggatcatattcttccggagaaccagtgctgcagggtctgccgaggtcataacttctgtgcagaagcacctaagtgtggagaaaactcggaatgcaaaaattggaatacaaaagcgacttgtgagtgcaagaatggatacatctctgtccagggcaactctgcatactgtgaagatatcgatgagtgtgcagcaaagatgcactactgtcatgccaacacggtgtgtgtcaacttgccggggttatatcgctgtgactgcatcccaggatacatccgtgtggatgacttctcttgtacggagcatgatgattgtggcagcggacaacacaactgtgacaaaaatgccatctgtaccaacacagtccagggacacagctgtacctgccagccaggctacgtgggaaatggtactgtctgcaaagcattctgtgaagagggttgcagatacggaggtacctgtgtggcccctaacaaatgtgtctgtccttctggattcacaggaagccactgtgagaaagatattgatgaatgtgcagagggattcgttgagtgccacaaccactcccgctgcgttaaccttccagggtggtaccactgtgagtgcagaagcggtttccatgacgatgggacctattcactgtccggggagtcctgcattgatattgatgaatgtgccttaagaactcacacttgttggaatgactctgcctgcatcaacttagcaggaggatttgactgcctgtgtccctctgggccctcctgctctggtgactgtccccacgaaggggggctgaagcataatgggcaggtgtggattctgagagaagacaggtgttcagtctgttcctgtaaggatgggaagatattctgccggcggacagcttgtgattgccagaatccaaatgttgaccttttctgctgcccagagtgtgacaccagggtcactagccaatgtttagatcaaagcggacagaagctctatcgaagtggagacaactggacccacagctgccagcagtgccgatgtctggaaggagaggcagactgctggcctctagcttgccctagtttgagctgtgaatacacagccatctttgaaggagagtgttgtccccgctgtgtcagtgacccctgcctggctgataatattgcctatgacatcagaaaaacttgcctggacagctctggtatttcgaggctgagcggcgcagtgtggacaatggctggatctccctgtacaacctgtcaatgcaagaatgggagagtctgctgctctgtggatctggtgtgtcttgagaataactgaagattttaaatggactcatcacatgagaaaatggacaaaatgaccatccaacctgaggaagaggaggggctgatttctttttctttttaaccacagtcaattaccaaagtctccatcagaggaaggcgtttgggttgcctttaccactttgctcatccttgctgacctagtctagatgcctgcagtaccgtgtatttcggtcgatggttgttgagtctccgtgctgtaaatcacatttcccttgtcagatcatttacagatacatttaaaggattccatgataaatgttaaagtaccttttgtttattttgtgtaccaacataatagagacttggcacca

SEQ ID NO: 4 - protein, 810 aa, Mus musculus
MPMDVILVLWFCVCTARTVLGFGMDPDLQMDIITELDLVNTTLGVTQVAGLHNASKAFLFQDVQREIHSAPHVSEKLIQLFRNKSEFTFLATVQQKPSTSGVILSIRELEHSYFELESSGPREEIRYHYIHGGKPRTEALPYRMADGQWHKVALSVSASHLLLHVDCNRIYERVIDPPETNLPPGSNLWLGQRNQKHGFFKGIIQDGKIIFMPNGFITQCPNLNRTCPTCSDFLSLVQGIMDLQELLAKMTAKLNYAETRLGQLENCHCEKTCQVSGLLYRDQDSWVDGDNCRNCTCKSGAVECRRMSCPPLNCSPDSLPVHISGQCCKVCRPKCIYGGKVLAEGQRILTKTCRECRGGVLVKITEACPPLNCSEKDHILPENQCCRVCRGHNFCAEAPKCGENSECKNWNTKATCECKNGYISVQGNSAYCEDIDECAAKMHYCHANTVCVNLPGLYRCDCIPGYIRVDDFSCTEHDDCGSGQHNCDKNAICTNTVQGHSCTCQPGYVGNGTVCKAFCEEGCRYGGTCVAPNKCVCPSGFTGSHCEKDIDECAEGFVECHNHSRCVNLPGWYHCECRSGFHDDGTYSLSGESCIDIDECALRTHTCWNDSACINLAGGFDCLCPSGPSCSGDCPHEGGLKHNGQVWILREDRCSVCSCKDGKIFCRRTACDCQNPNVDLFCCPECDTRVTSQCLDQSGQKLYRSGDNWTHSCQQCRCLEGEADCWPLACPSLSCEYTAIFEGECCPRCVSDPCLADNIAYDIRKTCLDSSGISRLSGAVWTMAGSPCTTCQCKNGRVCCSVDLVCLENN

SEQ ID NO: 5 - DNA, 2915 bp, Rattus norvegicus
  CDS (59)..(2491)
  sig_peptide (59)..(121)
aagcactggtttcttgttagcgttggtgcgccctgcttggcgggggttctccggagcgatgccgatggatgtgattttagttttgtggttctgtgtatgcaccgccaggacagtgttgggctttgggatggaccctgaccttcagctggacatcatctcagagctcgacctggtgaacaccaccctgggagtcacgcaggtggctggactgcacaacgccagtaaagcatttctatttcaagatgtacagagagagatccattcggcccctcacgtgagtgagaagctgatccagctattccggaataagagcgagttcacctttttggctacagtgcagcagaaaccatccacctcaggggtgatactgtccatccgggagctggagcacagctattttgaactggagagcagtggcccaagagaagagatacgctaccattacatacatggtggaaagcccaggactgaggcccttccctaccgcatggcagacggacaatggcacaaggtcgcgctgtcagtgagcgcctctcacctcctgctccacatcgactgcaataggatttacgagcgtgtgatagaccctccggagaccaaccttcctccaggaagcaatctgtggcttgggcaacgtaaccaaaagcatggctttttcaaaggaatcatccaagatggtaagatcatcttcatgccgaatggtttcatcacacagtgtcccaacctcaatcgcacttgcccaacatgcagtgacttcctgagcctggttcaaggaataatggatttgcaagagcttttggccaagatgactgcaaaactgaattatgcagagacgagacttggtcaactggaaaattgccactgtgagaagacctgccaagtgagtgggctgctctacagggaccaagactcctgggtggatggtgacaactgtgggaactgcacgtgcaaaagtggtgccgtggagtgccgcaggatgtcctgtcccccgctcaactgttccccggactcacttcctgtgcacatttccggccagtgttgtaaagtttgcagaccaaaatgtatctatggaggaaaagttcttgctgagggccagcggattttaaccaagacctgccgggaatgtcgaggtggagtcttggtaaaaatcacagaagcttgccctcctttgaactgctcagcaaaggatcatattcttccagagaatcagtgctgcagggtctgcccaggtcataacttctgtgcagaagcacctaagtgcggagaaaactcggaatgcaaaaattggaatacaaaagcaacctgtgagtgcaagaatggatacatctctgtccagggcaactctgcatactgtgaagatattgatgagtgtgcagctaaaatgcactattgtcatgccaacaccgtgtgtgtcaacttgccggggttgtatcgctgtgactgcgtcccagggtacatccgtgtggatgacttctcttgtacggagcatgatgattgtggcagcggacaacacaactgcgacaaaaatgccatctgtaccaacacagtccagggacacagctgcacctgccagccgggttacgtgggaaatggcaccatctgcaaagcattctgtgaagagggttgcagatacggaggtacctgtgtggctcctaacaagtgtgtctgtccttctggattcacgggaagccactgtgagaaagatattgatgaatgcgcagagggattcgttgaatgccacaactactcccgctgtgttaacctgccagggtggtaccactgtgagtgcagaagcggtttccatgacgatgggacctactcactgtccggggagtcctgcattgatatcgatgaatgtgccttaagaactcacacttgttggaatgactctgcctgcatcaacttagcaggaggatttgactgcctgtgtccctctgggccctcctgctctggtgactgtccccacgaaggagggctgaagcataatgggcaggtgtggattctgagagaagacaggtgttcagtctgttcctgcaaggatgggaagatattctgccggcggacagcttgtgattgccagaatccaaatgttgaccttttttgctgcccagagtgcgataccagggtcaccagccaatgtttagatcaaagtggacagaagctctatcgaagtggagacaactggacccacagctgccagcagtgccgatgtctggaaggagaggcagactgctggcctctggcttgccctagtttgggctgtgaatacacagccatgtttgaaggggagtgttgtccccgatgtgtcagtgacccctgcctggctggtaatattgcctatgacatcagaaaaacttgcctggacagctttggtgtttcgaggctgagcggagccgtgtggacaatggctggatctccttgtacaacctgcaaatgcaagaatgggagagtctgctgctctgtggatctggagtgtattgagaataactgaagattttaaatggactcgtcacgtgagaaaatgggcaaaatgatcatcccacctgaggaagaagaggggctgatttctttttctttttaaccacagtcaattaccaaagtctccatctgaggaaggcgtttggattgcctttgccactttgctcatccttgctgacctagtctagatgcctgcagtaccgtgcatttcggtcgatggttgttgagtctcagtgttgtaaatcgcatttccctcgtcagatcatttacagatacatttaaaggggttccatgataaatgttaatgtaacttttgtttattttgtgtactgacataatagagacttggcaccatttatttatttttcttgatttttggatcaaattctaaaaataaagttgcctgttgcgaaaaaaaaaaaaaaaaaaaaaaaaaa

SEQ ID NO: 6 - protein, 810 aa, Rattus norvegicus
MPMDVILVLWFCVCTARTVLGFGMDPDLQLDIISELDLVNTTLGVTQVAGLHNASKAFLFQDVQREIHSAPHVSEKLIQLFRNKSEFTFLATVQQKPSTSGVILSIRELEHSYFELESSGPREEIRYHYIHGGKPRTEALPYRMADGQWHKVALSVSASHLLLHIDCNRIYERVIDPPETNLPPGSNLWLGQRNQKHGFFKGIIQDGKIIFMPNGFITQCPNLNRTCPTCSDFLSLVQGIMDLQELLAKMTAKLNYAETRLGQLENCHCEKTCQVSGLLYRDQDSWVDGDNCGNCTCKSGAVECRRMSCPPLNCSPDSLPVHISGQCCKVCRPKCIYGGKVLAEGQRILTKTCRECRGGVLVKITEACPPLNCSAKDHILPENQCCRVCPGHNFCAEAPKCGENSECKNWNTKATCECKNGYISVQGNSAYCEDIDECAAKMHYCHANTVCVNLPGLYRCDCVPGYIRVDDFSCTEHDDCGSGQHNCDKNAICTNTVQGHSCTCQPGYVGNGTICKAFCEEGCRYGGTCVAPNKCVCPSGFTGSHCEKDIDECAEGFVECHNYSRCVNLPGWYHCECRSGFHDDGTYSLSGESCIDIDECALRTHTCWNDSACINLAGGFDCLCPSGPSCSGDCPHEGGLKHNGQVWILREDRCSVCSCKDGKIFCRRTACDCQNPNVDLFCCPECDTRVTSQCLDQSGQKLYRSGDNWTHSCQQCRCLEGEADCWPLACPSLGCEYTAMFEGECCPRCVSDPCLAGNIAYDIRKTCLDSFGVSRLSGAVWTMAGSPCTTCKCKNGRVCCSVDLECIENN

SEQ ID NO: 7 - DNA, 3336 bp, Artificial Sequence
  synthetic vector
ggatcatgatgataaacaatgtatggtgctaatgttgcttcaacaacaattctgttgaactgtgttttcatgtttgccaacaagcacctttatactcggtggcctccccaccaccaacttttttgcactgcaaaaaaacacgcttttgcacgcgggcccatacatagtacaaactctacgtttcgtagactattttacataaatagtctacaccgttgtatacgctccaaatacactaccacacattgaacctttttgcagtgcaaaaaagtacgtgtcggcagtcacgtaggccggccttatcgggtcgcgtcctgtcacgtacgaatcacattatcggaccggacgagtgttgtcttatcgtgacaggacgccagcttcctgtgttgctaaccgcagccggacgcaactccttatcggaacaggacgcgcctccatatcagccgcgcgttatctcatgcgcgtgaccggacacgaggcgcccgtcccgcttatcgcgcctataaatacagcccgcaacgatctggtaaacacagttgaacagcatctgttcgaatttaaagcttggtaccgagctcggatccactagtccagtgtggtggaattctgcagatatccagcacagtggcggccgctcgagtctagagggcccgcggttcgaaggtaagcctatccctaaccctctcctcggtctcgattctacgcgtaccggtcatcatcaccatcaccattgagtttatctgactaaatcttagtttgtattgtcatgttttaatacaatatgttatgtttaaatatgtttttaataaattttataaaataatttcaacttttattgtaacaacattgtccatttacacactcctttcaagcgcgtgggatcgatgctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcgaaacacgcacggcgcgcgcacgcagcttagcacaaacgcgtcgttgcacgcgcccaccgctaaccgcaggccaatcggtcggccggcctcatatccgctcaccagccgcgtcctatcgggcgcggcttccgcgcccattttgaataaataaacgataacgccgttggtggcgtgaggcatgtaaaaggttacatcattatcttgttcgccatccggttggtataaatagacgttcatgttggtttttgtttcagttgcaagttggctgcggcgcgcgcagcacctttgccgggatctgccgggctgcagcacgtgttgacaattaatcatcggcatagtatatcggcatagtataatacgacaaggtgaggaactaaaccatggcggtagaaaaaatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgtcacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaccgacgccgaccaacaccgccggtccgacgcggcccgacgggtccgaggggggtcgacctcgaaacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 8 - DNA, 69 bp, Artificial Sequence
  synthetic tag
ggtaagcctatccctaaccctctcctcggtctcgattctacgcgtaccggtcatcatcaccatcaccat

SEQ ID NO: 9 - protein, 23 aa, Artificial Sequence
  synthetic tag
GKPIPNPLLGLDSTRTGHHHHHH

SEQ ID NO: 10 - protein, 21 aa, Homo sapiens
MPMDLILVVWFCVCTARTVVG

SEQ ID NO: 11 - protein, 21 aa, Honey Bee
  Mellitin
MKFLVNVALVFMVVYISYIYA

SEQ ID NO: 12 - DNA, 3511 bp, Xenopus sp.
  CDS (1)..(2487)
cactttatgtttagcggctgccgggggattattggggctccttctccgctcactgcgccaatggatggaatgctggctgtgtgggcttgggtactgactgccagagcagtgctcggattggggacagaccccgacctacagatcgacgtaataacggaactggaactggtcaattcaacgagaggcgtgacgcaagtattcggggtgcacaatgggagcaaagcgtttttattccaagacggggcgcgagagatccacgcggcgccgcacatgagcgagaaagtcatccagttgttcaggaacaaaagcgaattcactttcttggcctccatccagcaacgctcctccacgtcgggggtcatcctctccatccgcgagatggagcacagtttcttcgagctggagagcagcggcctgcgggatgaaatccgatatcattaccgcttcaacgggaagtcgaggacggaggcctttccctaccgccttgccgacgggcagtggcacaaaatcgccctcaccgtcagcgcctcccacgttctgctgcatgtggactgcaataggatttacgaacgtgtaatagacccgccggacgccaatctctcgccgggaagcagcctgtggctcggacagcgcaaccgcaagcacggattctttaagggagtcattcaagatgtaaaaatgatcttcatgcccaatggatacatcactcagtgcccgaacttaaatcgcacttgcccaacctgcagcgatttcctgagtctcgtgcaaggaattatggatctacaggagctgctggcgaaaatgacagcaaaattgaactatgcagaaacccggctcagccaactggagaactgccattgtgaaaagacgtgtaacgtccgggaggtggtctatagggacagggactcgtgggtggatgacgatcactgccggaactgtacttgtaagaatggggcagttgagtgccggcggatgctgtgcccccctcttaattgttcctccgattctctcccggtccatgtcgcggggcagtgctgcaaagtctgcaggcccaagtgtatctatggagggaaagtgctggcggaaggagaccggatcctcaccaaaagctgccgagaatgcaaaaatggatctttacaaaagctgatggacccttgcccgcccctcaactgctccgaagccgaccgggttttgccagaaaatcagtgctgcagtgtttgcagagggcataacttctgtgccgacggacacaggtgcggtgagaactcggaatgtaggaaccataacaacaaagctacgtgtgagtgtctcagtggctttcaacccatccaaggggatccggcctactgtgaggatattgatgaatgtgcagccaagatgcattactgtcacgccaatacggtctgcgtcaatttaccgggatcttaccggtgtgattgtgtgccaggatacatccgagtcgatgatttctcctgcacagaacatgatgaatgcggcgaaggtgagagcagctgtgacgagaatgccatctgcgccaactccgtgcgagggcacagctgcacctgtaagccggggtatgtcgggaacggcaccgtctgccgggctttttgcgaggaagggtgtcggtacggcgggacctgtgtggcccccaacaaatgtctctgcccctccggattcaccgggagccactgtgaaaaagatattgatgagtgtgcagaagggatcattgagtgtcacaaccattctcgctgtgttaacctcccgggctggttccactgtgagtgccggagcggtttccatgacaatgggacatactccatttctggggagtcctgtgttgacattgatgaatgcgccctaagaactcacacttgttggaacgactcggcttgcgtcaatctcgagggcggcttcgattgcctttgcccttccggaccctcctgcacaggggactgcccccacgaggggggactcaagcgcaatgggcaggtctggacactgaaagaggatcggtgctcagtctgttcatgcaaggatgggaaaatattctgccgaagaaccgcctgcgactgccagaatcccagcgtcgatctgttctgctgccccgagtgcgacacgagagtcactagccaatgcctggatcagactggccacaaggtgtatcatagcggggacaactggacctacagctgccagcagtgccgctgcctggagggagaagtcgactgttggccgctgacctgccccattctgacctgcgagtacacgaccatctcagaaggcgagtgctgccctcactgcgtcgatgacccctgtatagcagacggggacccctatgacatcaggaaaacctgtcaggaccctcaaggaattacacggctggggggctcagtatggacgatggtcgggtcgccgtgtaccacctgtaaatgcaagaacggaagtgtttgctgctcagtggatttggactgtcttcacaataactaaagttctaacccagtggactctctaatggagaggaagtctaatgcagtcccccatctacagcagagtctccgtccgcagctctgtttcctacaacagacaattaccaaagtctttaccaagaggaagatgtttgggagtttcttgtggaccttcgtctatgatcacaccctcggccctcgcctgggcgccacacatacaaggccgacggctcgttctgagcaacctggggggccgccatgcatctctagctccctccaatggcgattgtttgctcattctcagtgttgtgaaccacttttattctcgttacacttttattttctttattgtagacctttgaatttttcccccaacccgtccaccaccaccaccattttttgtatggcatccaccaccatttttttaaaacttgggttccagaatctaccgaatagtcggatctcatccattgcaatctactaaggccccccctgtctgatgtatttatgaaaatatgtatgtatgtacagtctaattgcatagtaattatatttcatagctatggaacattgtacaattcagcccaaacctgtctctcgagaagaaacatcacgagctgcttcttcatcccaaagggaaccttattggacaatttccggatcacatggtccacattgctttgcttcaggaccttcatggatcatgttgggaaatgataaagtccttcctcctttccccacaatagaacaatagagactttgaaggacttacctggtgcacgtggccgagacgtccagagcgtcgctgcgagacctccggggatgcaacagagatacgacatgctgtgctattgcccatcgttatttatccatttattgaggaaaattttcacgggggttgttatttttaaacaaaaaaatagagagtggaaaagtgatgactgtaagtacggaagcgccagagcggcattactggcgggattattttcagtttactttttaatcgatcacaataaaagccttttaccatcaaaaaaaaaaaaaaa

SEQ ID NO: 13 - protein, 828 aa, Xenopus sp.
HFMFSGCRGIIGAPSPLTAPMDGMLAVWAWVLTARAVLGLGTDPDLQIDVITELELVNSTRGVTQVFGVHNGSKAFLFQDGAREIHAAPHMSEKVIQLFRNKSEFTFLASIQQRSSTSGVILSIREMEHSFFELESSGLRDEIRYHYRFNGKSRTEAFPYRLADGQWHKIALTVSASHVLLHVDCNRIYERVIDPPDANLSPGSSLWLGQRNRKHGFFKGVIQDVKMIFMPNGYITQCPNLNRTCPTCSDFLSLVQGIMDLQELLAKMTAKLNYAETRLSQLENCHCEKTCNVREVVYRDRDSWVDDDHCRNCTCKNGAVECRRMLCPPLNCSSDSLPVHVAGQCCKVCRPKCIYGGKVLAEGDRILTKSCRECKNGSLQKLMDPCPPLNCSEADRVLPENQCCSVCRGHNFCADGHRCGENSECRNHNNKATCECLSGFQPIQGDPAYCEDIDECAAKMHYCHANTVCVNLPGSYRCDCVPGYIRVDDFSCTEHDECGEGESSCDENAICANSVRGHSCTCKPGYVGNGTVCRAFCEEGCRYGGTCVAPNKCLCPSGFTGSHCEKDIDECAEGIIECHNHSRCVNLPGWFHCECRSGFHDNGTYSISGESCVDIDECALRTHTCWNDSACVNLEGGFDCLCPSGPSCTGDCPHEGGLKRNGQVWTLKEDRCSVCSCKDGKIFCRRTACDCQNPSVDLFCCPECDTRVTSQCLDQTGHKVYHSGDNWTYSCQQCRCLEGEVDCWPLTCPILTCEYTTISEGECCPHCVDDPCIADGDPYDIRKTCQDPQGITRLGGSVWTMVGSPCTTCKCKNGSVCCSVDLDCLHNN

SEQ ID NO: 14 - protein, 90 aa, Homo sapiens
SEFTILATVQQKPSTSGVILSIRELEHSYFELESSGLRDEIRYHYIHNGKPRTEALPYRMADGQWHKVALSVSASHLLLHVDCNRIYERV

SEQ ID NO: 15 - protein, 89 aa, Homo sapiens
PEFKLVFSIRPRSLTGILIHIGSQPGKHLCVYLEAGKVTASMDSGAGGTSTSVTPKQSLCDGQWHSVAVTIKQHILHLELDTDSSYTAG

SEQ ID NO: 16 - protein, 89 aa, Mus musculus
PALTLTLSIRPRSLTGVLIHIASQSGEHLSVYMEAGKVTTSMNSEAGGTVTSITPKRSLCDGQWHSVTVSIKQHTLHLELDTYNSYTAG

SEQ ID NO: 17 - protein, 93 aa, Homo sapiens
EDFSILFTVKPKKGIQSFLLSIYNEHGIQQIGVEVGRSPVFLFEDHTGKPAPEDYPLFRTVNIADGKWHRVAISVEKKTVTMIVDCKKKTTKP

SEQ ID NO: 18 - protein, 93 aa, Homo sapiens
KGFLLLASLRQMKKTRGTLLALERKDHSGQVFSVVSNGKAGTLDLSLTVQGKQHVVSVEEALLATGQWKSITLFVQEDRAQLYIDCEKMENAE

SEQ ID NO: 19 - DNA, 33 bp, Artificial Sequence
  synthetic forward primer#2
ggaagcttcggagcgatgccgatggatttgatt

SEQ ID NO: 20 - DNA, 21 bp, Artificial Sequence
  synthetic forward primer#3
tcagttagcgcctctcatctc

SEQ ID NO: 21 - DNA, 21 bp, Artificial Sequence
  synthetic forward primer#4
gcggattttaaccaagagctg

SEQ ID NO: 22 - DNA, 22 bp, Artificial Sequence
  synthetic forward primer#5
gtgtctgtccatctggattcac

SEQ ID NO: 23 - DNA, 37 bp, Artificial Sequence
  synthetic reverse primer#2
gtaaccggttcaattattttgaagacactcaaaatcc

SEQ ID NO: 24 - DNA, 21 bp, Artificial Sequence
  synthetic reverse primer#3
atctttctcgcagtggcttcc

SEQ ID NO: 25 - DNA, 21 bp, Artificial Sequence
  synthetic reverse primer#4
ctaaaactccacctcggcatt

SEQ ID NO: 26 - DNA, 21 bp, Artificial Sequence
  synthetic reverse primer#5
atcctgttacagtcgacatgg

SEQ ID NO: 27 - protein, 12 aa, Artificial Sequence
  synthetic tag
PIPNPLLGLDST

SEQ ID NO: 28 - protein, 6 aa, Artificial Sequence
  synthetic tag
HHHHHH

SEQ ID NO: 29 - protein, 27 aa, Artificial Sequence
  synthetic tag
PRFEGKPIPNPLLGLDSTRTGHHHHHH

SEQ ID NO: 30 - DNA, 21 bp, Artificial Sequence
  synthetic forward primer#1
ggctcatttgcttccacctag

SEQ ID NO: 31 - DNA, 21 bp, Artificial Sequence
  synthetic reverse primer#1
gtcatttcgtccattcttctg